SEQUENCE LISTING (42 sequences)

SEQ ID NO: 1 - protein, 39 aa, Dionaea muscipula
ATTKLIVLLSSQLVVAAFIFGNTCKTIFEAIIFVFVMHP

SEQ ID NO: 2 - protein, 39 aa, Drosera capensis
MTTKVLLFFSSQLLVAVFVFGNTCKTIFEAIIFVFVMHP

SEQ ID NO: 3 - protein, 39 aa, Drosera capensis
MTTKVLLFFTSQLLVAVFVFGNTCKTIFEAIIFVFVMHP

SEQ ID NO: 4 - protein, 39 aa, Escherichia coli
QTASVIAVLGAAGLAVGLALQGSLSNLAAGVLLVMFRPF

SEQ ID NO: 5 - protein, 752 aa, Dionaea muscipula
MGSYLHEPPGDEPSMRIEQPKTADRAPEQVAIHICEPSKVVTESFPFSETAEPEAKSKNCPCPEIARIGPCPNKPPKIPINRGLSRISTNKSRPKSRFGEPSWPVESSLDLTSQSPVSPYREEAFSVENCGTAGSRRGSFARGTTSRAASSSRKDETKEGPDEKEVYQRVTAQLSARNQKRMTVKLMIELSVFLCLLGCLVCSLTVDGFKRYTVIGLDIWKWFLLLLVIFSGMLITHWIVHVAVFFVEWKFLMRKNVLYFTHGLKTSVEVFIWITVVLATWVMLIKPDVNQPHQTRKILEFVTWTIVTVLIGAFLWLVKTTLLKILASSFHLNRFFDRIQESVFHHSVLQTLAGRPVVELAQGISRTESQDGAGQVSFMEHTKTQNKKVVDVGKLHQMKQEKVPAWTMQLLVDVVSNSGLSTMSGMLDEDMVEGGVELDDDEITNEEQAIATAVRIFDNIVQDKVDQSYIDRVDLHRFLIWEEVDHLFPLFEVNEKGQISLKAFAKWVVKVYNDQAALKHALNDNKTAVKQLNKLVTAILIVMMIVIWLIVTGIATTKLIVLLSSQLVVAAFIFGNTCKTIFEAIIFVFVMHPFDVGDRCVIDGNKMLVEEMNILTTVFLKWDKEKVYYPNSILCTKAIGNFFRSPDQGDVLEFSVDFTTPVLKIGDLKDRIKMYLEQNLNFWHPQHNMVVKEIENVNKIKMALFVNHTINFQDFAEKNRRRSELVLELKKIFEELDIKYNLLPQEISIRNM

SEQ ID NO: 6 - DNA, 2259 bp, synthetic construct
  misc_feature 1..2259: note = Description of artificial sequence: synthetic polynucleotide sequence
  source 1..2259: mol_type = other DNA
atgggatcctatttacatgagccccccggcgacgagccttccatgaggatcgagcagcctaagacagctgacagagctcccgagcaagttgccattcacatctgtgaaccttccaaagtcgtgaccgagtcctttcccttctccgagacagccgagcccgaggctaagtccaagaactgcccttgccccgaaattgccagaattggcccttgccctaacaaacctcccaagatccctatcaataggggtttatctcgtatctccaccaacaagagcagacctaagtccagattcggagagcccagctggcccgttgagagctctttagacctcacaagccagtcccccgtcagcccttatcgtgaggaagccttcagcgtcgaaaattgcggcacagccggctctcgtaggggatccttcgctaggggaacaaccagcagagccgcctccagctctcgtaaggatgaaacaaaggagggccccgatgaaaaggaagtgtaccagagggtgacagcccagctgagcgctagaaaccagaagaggatgaccgtcaagctgatgatcgaactgtccgtgtttttatgtctgctgggctgtctggtctgctctttaacagtggatggattcaagaggtacaccgtgatcggtttagacatctggaaatggtttttactgctgctggtcatcttcagcggaatgctgatcacacactggatcgtgcacgtcgccgtcttcttcgtggagtggaaatttctgatgagaaaaaacgtgctgtactttacccacggtttaaaaacctccgtcgaggtgttcatctggatcacagtcgtgctcgccacttgggtgatgctgattaaacccgacgtgaaccagccccaccaaactcgtaagattttagagtttgtgacttggaccatcgtgacagtcctcattggcgcctttttatggctggtgaaaaccacactcctcaaaattctggccagctccttccacctcaatagattcttcgatcgtatccaagaatccgtgtttcaccatagcgtgctccagacactcgccggcagacccgttgtggagctggctcaaggtattagcagaaccgagagccaagatggagccggacaagtttcctttatggagcacacaaagacccaaaacaagaaagtggtcgacgtgggcaagctgcaccagatgaagcaagaaaaggtgcccgcttggacaatgcagctgctcgtggatgtcgtgtccaactccggtttatccacaatgtccggcatgctggacgaagacatggtggagggcggagtggagctggatgacgatgagatcaccaacgaggaacaagctattgccaccgccgttcgtatcttcgacaatatcgtgcaagataaggtggaccagagctacattgacagagtcgacctccataggtttttaatctgggaggaggtcgatcatttattccctttattcgaggtcaatgagaagggccaaatctctttaaaggcctttgccaagtgggtcgtcaaggtgtacaatgatcaagccgctttaaagcacgccctcaacgacaacaagaccgccgtgaagcaactgaataagctcgtgaccgctattctgattgtgatgatgatcgtgatttggctgatcgtcaccggcatcgccacaacaaaactgatcgtgctgctgagcagccagctcgtcgtcgctgcctttatcttcggcaacacttgtaaaacaatcttcgaggccatcatcttcgtcttcgtgatgcacccctttgacgtgggagatcgttgtgtcatcgacggaaacaaaatgctcgtggaggagatgaacatcctcaccaccgtctttttaaaatgggataaagagaaggtctattaccccaactccattttatgcaccaaggctatcggcaatttctttcgtagccccgatcaaggcgacgttttagagttctccgtcgacttcacaacacccgttttaaaaattggcgatttaaaggacagaatcaagatgtacctcgaacagaatttaaatttctggcacccccaacacaacatggtggtcaaggagatcgagaacgtcaacaagattaagatggctctgttcgtgaaccacaccatcaacttccaagatttcgccgaaaaaaataggaggagatccgaactggttttagaactgaaaaagattttcgaagagctggacatcaagtacaacttattacctcaagaaatttccattcgtaatatgtga

SEQ ID NO: 7 - protein, 897 aa, Dionaea muscipula
MEGVRNPLRNSFNKAHEAEPQRKKNLEQEERLILLQHRNDPNSQSFSSEDPNSLLLQVKVEVAGSCDPAKTAVPTKPPVSPGGGGNLIWRDSSYDFRNDVVKGCSRDTDDDSGEFDFQKHRVAEEEDEGEERDPESQTLSPVSESPHEYGKITPRGAAKVSFKESELVHRRPSDGGVFAADGVSASVRDEEVVMCTSNASCQRKSTSTRVKTKSRLLDPPDDGDTRSGRILRSGLMPRSEDHEDEDPFSGEDIPEEYKKMKFSFLSAVELVSLLLIIAGLVCSVVIPVVRRVTVWDMQLWKWEVMVLVLICGGLVSGWLIRFVVFFIERNFLLRKRVLYFVYGLRRAVQRCLWLGWVLIAWRLILDKKVEKETNSRSLLYVTKILVCLVVGTLIWLLKTLLVKVLAMSFHVSTFFDRIQEALFDQYVIETLSGPPTIEIQHVKEDEDQVMLEVQKLQSAGLSIPAELKATCLPNVNVNGKPVGSDPGPTPGVGKSPRSGVIGKSPRFSRAMPEKEEGAGGITIDHLHRLNQKNISAWNMKRLMNIVRYGVLSTLDEQILESGIEDEPSLHIKNENQAKAAAKRLFKNVARPGSKCIYLEDLMRFMREDEAARTMRAIEGSAESKGISKIALKNWVVNVFRERRALALSLNDTKTAVNKLHQLLNFIVGFTIAIIWLLILGVPMTHFFVFITSQLLLLTFMFGNTFKTTFEAIIFLFVMHPFDVGDRCEVEGVQMIVEEMNILTTVFLRYDNLKITYPNSVLATKPINNYYRSPEMGDSVDFCVHISTPVEKIVVMKERITRYMESRRDHWRPSPKVVMREVEDMNRLKFSVWMCHTMNHQDMGERWARRELLVVEMVKIFKELDVQYRMLPHDVNVRTMPSLVCDRLPSNWITCTGK

SEQ ID NO: 8 - DNA, 2694 bp, synthetic construct
  misc_feature 1..2694: note = Description of artificial sequence: synthetic polynucleotide sequence
  source 1..2694: mol_type = other DNA
atggagggcgttcgtaaccctttaaggaactccttcaacaaggcccacgaagccgagcctcagaggaagaagaatctggaacaagaagagaggctgattttactgcaacataggaacgaccccaactcccagagcttcagctccgaagatcctaactctttactgctgcaagttaaggtggaggtggccggaagctgcgatcccgctaaaaccgccgtgcccacaaaaccccccgttagccccggtggcggcggaaatttaatctggagagacagcagctacgactttcgtaatgacgtggtgaagggctgctccagagacaccgacgacgactccggcgaattcgacttccagaagcatcgtgtggccgaggaggaggacgaaggcgaagaaagagatcccgagtcccagacactcagccccgtttccgagtccccccacgagtatggcaaaatcacccccagaggagccgctaaagtgagcttcaaggagagcgagctggtgcacagaaggcctagcgatggcggagtgtttgccgccgatggagtgagcgccagcgtgagggacgaggaggtcgtgatgtgcacctccaacgcctcttgtcagaggaagagcacaagcactcgtgtgaaaaccaagtctcgtctgctcgatcctcccgatgacggcgacaccagatccggtcgtattttacgttccggtttaatgcccagaagcgaagatcacgaggacgaagaccccttttccggcgaagacatccccgaagagtataagaagatgaagtttagctttttatccgctgtggagctggtctctttattattaatcatcgccggcctcgtctgcagcgtcgtgattcccgtggtgaggagggtcaccgtgtgggatatgcagctctggaagtgggaagtgatggtgctcgttttaatctgcggcggtttagtcagcggatggctgatcagatttgtggtctttttcatcgaaagaaactttttactgaggaagagggtgctctatttcgtgtacggtttaagaagggctgtgcaaaggtgcctctggctgggctgggtgctgatcgcttggagactgatcctcgacaagaaggtcgagaaggagaccaattctcgtagcttattatatgtcaccaagattttagtctgtttagtggtgggcactttaatctggctgctgaaaactttactggtgaaggttttagccatgtcctttcatgtgagcaccttcttcgatcgtattcaagaagctttattcgaccagtacgtcattgaaacactctccggcccccccacaattgagatccagcatgtcaaggaagacgaggaccaagttatgctggaggtgcagaaactgcaaagcgccggtttaagcattcccgctgagctcaaggccacttgtttacccaatgtgaatgtcaatggcaagcccgttggcagcgatcccggtcctacacccggcgtgggaaagtcccctagatccggagtgattggaaagagccctcgtttctctcgtgccatgcccgagaaggaggaaggagccggcggcatcaccatcgatcacctccacagactcaaccagaagaacatctccgcttggaatatgaagagactgatgaacatcgtgaggtatggcgttttatccacactggacgaacagatcctcgagagcggcatcgaagacgaaccctctttacatatcaagaacgagaaccaagccaaggctgccgccaagaggctcttcaaaaatgtcgctcgtcccggtagcaaatgcatctatctggaggacctcatgagattcatgagggaagatgaggccgctagaacaatgagggctatcgagggttctgccgagtccaaaggcatctccaagatcgccctcaagaattgggtcgtgaatgtgttcagagagaggagggctttagctttatctttaaatgacaccaagaccgccgtgaacaagctgcaccagttattaaacttcatcgtgggcttcacaatcgccatcatctggctcctcattttaggcgtgcccatgacccacttctttgtgttcattaccagccagttattactcctcaccttcatgttcggaaatacattcaaaacaacatttgaggccattatttttctgtttgtcatgcatcctttcgacgtgggcgacagatgtgaggtggaaggagtgcagatgatcgtggaagagatgaacattttaaccaccgtctttttaaggtacgacaatttaaagatcacctatcccaatagcgttttagctaccaagcccatcaacaattattacagaagccccgaaatgggagacagcgtcgacttctgcgtccacatctccacccccgtcgaaaagattgtcgtcatgaaggagaggatcacaaggtacatggagtctcgtagggaccactggagaccttcccccaaagtggtcatgagggaggtggaggatatgaatagactcaagttctccgtctggatgtgccacacaatgaaccaccaagacatgggcgagagatgggctcgtagggagctgctggtcgtggagatggtcaagatcttcaaggagctcgacgtgcagtatcgtatgctgccccacgatgtgaacgtgagaaccatgcccagcctcgtgtgcgacagactgcctagcaattggatcacatgcaccggaaaatga

SEQ ID NO: 9 - protein, 754 aa, Dionaea muscipula
MESNPEYIASLGDIVVAAVINIFFAFVFFIAFAIFRIQPVNDRVYYTKWYLRGLRSSSTNPDAFVRKCVNLSFGSYLKFLNWMPAALQMPETELIQHAGLDSAVYLRIYLVGLKIFIPITILALSIVIPVNWTDGGLEKSKLIAFNNLDKLSISNIRPGSEKFWTHIGMAYTVTFWACYILKKEYESIESMRLQFLASSGRKPEQFTVLVRNVPLDSDESTSELVEHFFKVNHPDDYLTRQVIYDANVLTDLVRERKKKQMWLNFYQLKYTRSQSRKPFCKTGFLGLWGTKVDAIDYYTMEVERLSKEISSKREMIANDTKAVMLAAFVSFKTRRGAAICAHTQQARNPTLWLTQWAPEPRDIYWRNLAIPYASLSIRKLIVSVTFFFLATFFMIPIAFVQSLANIEGIEKALPFLRPVIEARFVKSIIQGFLPGIVLKIFLTFLPSILMMMCKSEGIISLSALERRAAARYYVFLLINVFLGSIVTGTAFEQLNNILHETANAIPETIGAAIPMKVTFFITYTMVDGWAGMAAEILRLKPLICYHLKVCFLVNTEKDKEEAMNPQSFGFNTREPQIQLYFLVALVYAVAAPILLPFIVLLFSLGYIVYRHQIINVYNQEYESGAAFWPDVHKRIVVALVVSQLLLLGLLSTKKASHSTPLLVALPVLTISFHYLCKGRFLPAFVTHPLQEATLKDSMDLAREPGLHFKRYLQNAYTHPLLKVGDNAETDEAFQEVEQGCQLVQTKRQLWRTFS

SEQ ID NO: 10 - DNA, 2265 bp, synthetic construct
  misc_feature 1..2265: note = Description of artificial sequence: synthetic polynucleotide sequence
  source 1..2265: mol_type = other DNA
atggagagcaaccccgaatatattgctagcctcggcgatatcgtggtcgctgccgtcatcaacatcttcttcgcctttgtgttttttatcgcttttgccatcttcagaatccagcccgtgaacgatagagtgtactacaccaagtggtatctgagaggactgaggtcctccagcacaaaccccgacgcctttgtgaggaagtgcgtgaatctgagctttggcagctatctgaagtttctgaactggatgcccgccgccctccagatgcccgagacagagctgattcagcatgctggactcgattccgccgtgtacctcagaatctatctcgtcggactgaagatcttcatccccatcaccattctggccctcagcatcgtgattcccgtgaactggaccgatggcggcctcgagaagtccaagctgattgcttttaacaacctcgacaagctgtccatctccaacattagacccggaagcgagaagttttggacccacatcggcatggcctataccgtcaccttctgggcttgctatattctgaaaaaagagtacgagagcatcgaaagcatgaggctccagtttctcgccagcagcggaaggaagcccgagcagtttaccgtgctggtgaggaacgtccctctggatagcgatgaatccaccagcgaactggtcgaacacttcttcaaggtgaaccaccccgatgactatctgacaaggcaagtgatttacgacgccaacgtgctgaccgacctcgtgagggagaggaagaaaaagcagatgtggctcaacttctaccagctgaagtacacaagaagccagtctagaaagcccttttgcaagaccggcttcctcggactgtggggcacaaaggtggacgccatcgactactacacaatggaggtggagagactcagcaaggagatcagctccaaaagggagatgatcgctaatgacaccaaggctgtcatgctcgccgccttcgtcagctttaagacaaggaggggcgctgccatttgcgctcatacacagcaagccagaaaccctaccctctggctgacacagtgggcccccgaacctagggacatctactggaggaatctggccatcccctacgcctctctgagcattagaaaactgatcgtgagcgtgaccttcttctttctggctaccttcttcatgatccccatcgctttcgtgcaatctctggccaacatcgaaggaatcgagaaagccctcccctttctgaggcccgtcattgaagctagattcgtgaagtccatcatccaaggctttctgcccggcatcgtgctcaagatttttctgacctttctccccagcattctgatgatgatgtgcaagagcgagggaatcatttctctgagcgctctcgagaggagagctgccgctagatactacgtctttctgctgattaacgtctttctgggcagcattgtgaccggcaccgccttcgaacaactcaacaacattctgcacgaaacagccaacgctatccccgagaccattggcgctgccatccccatgaaagtcaccttttttatcacctacaccatggtcgatggctgggccggcatggctgccgagatcctcagactcaaacctctgatctgttaccatctgaaggtgtgttttctggtgaacaccgagaaggacaaggaggaggctatgaatcctcagtccttcggcttcaacaccagagagccccaaatccagctctattttctggtggctctggtctacgctgtggctgcccccattctgctgccctttatcgtcctcctcttctccctcggctacatcgtctacagacatcagatcatcaatgtgtacaaccaagagtacgagtccggagccgctttctggcccgatgtgcataagaggattgtcgtggctctggtggtcagccagctgctgctgctcggactgctcagcaccaagaaagctagccattccacacctctgctggtggctctgcccgtgctgacaatctccttccactatctgtgtaagggcagatttctgcccgccttcgtgacacatcctctgcaagaggccacactgaaagactccatggatctggctagggagcccggactgcactttaagaggtatctgcagaacgcttacacccaccctctgctgaaggtgggcgataatgctgaaaccgacgaagccttccaagaggtggaacaaggctgccaactggtgcaaaccaaaaggcagctgtggagaacctttagctga

SEQ ID NO: 11 - protein, 754 aa, Drosera capensis
MASNTNISQQGGEINFEKQMAHRRRHEQLAIQIPVKTASQTFRFNEEVDTRSKFSPAPDITMFYPQPSPNKPPRVPNRTLTRRSTTLKTKPKSRFGEPSLPIDPAALWELAPNSPTPSFREATPSSNNHRFSVGRGSSFAKGVTPRVAASSQRGETTIEGPDEKEVYERVTAQLSARDKKRMTVKLLIELAIFLFVSGCLISSLTIHGLKVRKIYGLPIWRLFLFLLVILSGMLVTHWMIHVVVFLIEWKFLLKKNVVYFTHGLKTSVEVFIWITLILATWGLLIEPDVRHTNRIRNALDFITWTLLSLLLGSFLWLIKTIMIKTLAASFHLNRFFDRIQESIFHHYVLQTLSGRPVVELASGVLTRTETHNGMVSFTEHTKTHKEKKMVDMGKLHQMKQEKVPDWTMQLLVDVVSNSGLSTMSGILDEDMAEGGVELDDDEITSEEQAIATAVRIFYNIVKDKDDQSYIDRKDLHRFLICEEVDLVFPLFEVKDKDQINLKAFSKWVVKLFKERQALKHALNDNKTAVKQLDKLVTSILIVVIIAVWLLLTEIMTTKVLLFFSSQLLVAVFVFGNTCKTIFEAIIFVFVMHPFDVGDRCVVDGTMMLVEEMNILTTVFLKWDKEKVYYPNAVLSTKAIGNYYRSPDQVDSLEFSIDFRTPLSKIGEIKERIKKYLHQNPHLWHPNHNFVVKEIENVNKIKMQLIFNHTINFQEFPERMKRRSELVLELKKIFEELDIKYNLLPQEVILNKVSP

SEQ ID NO: 12 - DNA, 2265 bp, synthetic construct
  misc_feature 1..2265: note = Description of artificial sequence: synthetic polynucleotide sequence
  source 1..2265: mol_type = other DNA
atggctagcaacacaaatatttcccagcaaggcggcgagatcaacttcgaaaagcagatggcccacagaaggagacatgagcagctggccatccaaatccccgtgaaaaccgccagccagaccttcagattcaacgaggaagtggacacaagaagcaagttcagccccgcccccgacattaccatgttctacccccagcctagccccaacaaacctcctagggtgcccaataggacactcaccagaaggagcaccacactgaagaccaaacccaaatctagattcggcgaaccttctctgcctatcgatcccgctgccctctgggaactggctcccaattcccctacccccagctttagagaggccaccccctcctccaacaaccatagattctccgtgggaagaggcagcagctttgctaagggagtgacccctagagtcgccgccagcagccaaagaggcgagacaacaatcgagggccccgacgagaaagaagtgtacgagagggtcacagcccagctgagcgctagggataagaagaggatgaccgtcaagctgctgatcgagctggccatctttctgttcgtcagcggctgcctcatctccagcctcacaattcacggactgaaggtgagaaagatctacggactgcctatttggaggctgttcctctttctcctcgtcattctgagcggaatgctggtgacacactggatgatccatgtcgtggtgtttctgatcgaatggaagtttctgctgaagaagaatgtggtctacttcacccacggactgaagacctccgtggaggtcttcatttggatcacactgatcctcgccacatggggactgctcatcgagcccgacgtcagacataccaatagaattagaaatgccctcgacttcatcacatggacactgctgtctctgctgctcggcagctttctctggctgatcaagaccatcatgattaagacactcgccgcctccttccatctgaatagatttttcgatagaatccaagagtccatcttccaccactacgtgctgcagacactctccggcagacccgtcgtcgaactggcttccggagtgctgacaaggaccgagacacacaatggcatggtcagctttaccgagcacacaaagacccacaaggaaaagaagatggtggacatgggaaagctgcaccagatgaagcaagagaaggtccccgactggacaatgcagctgctggtggatgtcgtgagcaactccggcctcagcaccatgtccggaattctggacgaggacatggctgagggaggcgtggagctcgatgacgacgagatcacctccgaggagcaagccattgctaccgccgtcagaatcttctataatattgtcaaggacaaggacgaccagtcctacatcgacagaaaggacctccatagatttctgatctgtgaggaggtggatctggtgttccctctgtttgaggtgaaagacaaagaccagattaatctgaaggccttcagcaagtgggtcgtgaagctctttaaagagaggcaagccctcaagcacgccctcaacgacaacaagaccgccgtgaaacagctcgataagctggtgacctccattctgattgtggtgattatcgccgtgtggctgctgctgaccgagattatgaccaccaaagtgctgctgttcttctcctcccagctcctcgtggccgtgtttgtcttcggcaacacatgcaagacaatcttcgaggccattatctttgtgttcgtcatgcatcccttcgacgtgggcgatagatgtgtggtggacggcaccatgatgctggtcgaagagatgaacatcctcaccaccgtctttctgaagtgggacaaggaaaaggtgtactaccccaacgctgtcctctccaccaaggctattggcaattactatagaagccccgaccaagtggattctctggagttctccatcgactttagaacacccctctccaaaattggagagatcaaagagaggattaagaaatatctccatcagaacccccatctgtggcaccccaaccacaacttcgtggtgaaggagatcgaaaatgtcaataagattaagatgcagctgatctttaatcacacaattaatttccaagagtttcccgagaggatgaagaggagaagcgagctggtgctggagctgaagaagatcttcgaggagctggacatcaagtataatctgctgccccaagaggtcattctcaacaaggtgagcccttga

SEQ ID NO: 13 - protein, 755 aa, Drosera capensis
MASNTNISQQGGEINFEKQMAHRRRHEQLAIQIPVKTASQTFPFNEEVDTTRSKFSPAPDITMFYPQPSPNKPPRVPNRNLSRRSTTLKTKPKSRFGEPSLPIDPAALWELAPNSPAPSFREATPSSNNHRASVGRGSSFVKGVTPRVAASSRRGETTIEGPDEREVYERVTAQLSARDKKRMTVKLLIELAVFLFVSGCLISSLTIHGLKVRIICGLPIWRLFLFLLVILSGMLVTHWMLHVVVFLIEWKFLLKKNVVYFTHGLKTSVEVFIWITLILATWALLIEPDVRHTNRIRNALDFITWTLLSLLLCSFLWLIKTIMIKTLAASFHLNRFFDRIQESIFHHYVLQTLSGRPVVELASGVLTRTETHNGMVSFTEHTKTHTEKKMVDMGKLHQMKQEKVPDWTMQLLVDVVSNSGLSTMSGILDEDMAEGGVELDDDEITSEEQAIATAVRIFYNIVKDKDDQTYIDRKDLHRFLICEEVDLVFPLFEVKDKDQISLKAFSKWVVKLFKERQALKHALNDNKTAVKQLDKLVTSILIVVIIAVWLLLTELMTTKVLLFFTSQLLVAVFVFGNTCKTIFEAIIFVFVMHPFDVGDRCVIDGTTMLVEEMNILTTVFLKWDKEKVYYPNAVLSTKAIGNYYRSPDQVDSLEFSIDFRTPLSKIGEIKERIKKYLHQNPHLWHPNHNLVVKEIENVNKIKTQLIFNHTMNFQEFPERMKRRTELVLELKKIFEELDIKYNLLPQEVILNNVGP

SEQ ID NO: 14 - DNA, 2268 bp, synthetic construct
  misc_feature 1..2268: note = Description of artificial sequence: synthetic polynucleotide sequence
  source 1..2268: mol_type = other DNA
atggctagcaacacaaatatttcccagcaaggcggcgagatcaacttcgaaaagcagatggctcatagaagaaggcatgagcaactcgctatccagatccccgtgaagacagccagccagaccttccccttcaacgaggaagtcgataccacaagaagcaagttcagccccgcccccgacatcaccatgttctacccccaacctagccctaacaaaccccctagggtgcccaataggaatctgtctagaagatccaccacactgaagacaaagcccaagtctagattcggcgaacccagcctccctattgaccccgccgctctgtgggaactggcccccaacagccccgctccctcctttagggaagccacaccctccagcaacaaccacagagcctccgtgggaaggggcagctccttcgtgaagggagtcacacctagggtggccgccagctctagaagaggcgaaaccacaattgaaggccccgacgagagagaggtctatgagagagtgacagctcagctgagcgccagagataagaagagaatgaccgtgaagctcctcattgagctcgccgtctttctgtttgtgtccggatgtctgatctccagcctcacaattcacggactgaaggtgagaattatctgtggactgcccatctggagactgtttctgtttctgctggtgatcctctccggaatgctggtcacacactggatgctccatgtcgtcgtgttcctcatcgaatggaagtttctgctgaaaaagaatgtcgtgtacttcacccacggactgaaaacctccgtcgaggtgttcatctggatcacactgattctggccacatgggctctgctgattgagcccgacgtgagacacaccaatagaatcagaaacgccctcgacttcatcacatggacactgctgtctctgctgctctgctcctttctctggctgatcaagaccatcatgatcaagacactggccgcttccttccatctgaataggttctttgatagaatccaagagtccatcttccatcactacgtgctccagaccctctccggaaggcccgtggtcgagctcgcttccggcgtcctcaccagaacagagacccacaacggaatggtcagcttcaccgagcataccaagacccacacagagaagaagatggtggacatgggcaagctccaccaaatgaagcaagagaaggtccccgactggaccatgcagctgctggtggatgtggtgagcaatagcggactgagcaccatgtccggcattctggacgaagacatggccgaaggcggagtcgaactggacgatgacgagatcacctccgaagagcaagccattgccaccgctgtgaggattttctacaacatcgtcaaggacaaagacgaccagacctacatcgatagaaaggatctgcatagatttctgatctgcgaagaagtggacctcgtgttccccctctttgaagtgaaggacaaggaccagatctctctgaaggcctttagcaagtgggtggtgaagctcttcaaggagaggcaagccctcaagcacgccctcaacgacaataagaccgccgtcaagcagctggataaactcgtgacaagcatcctcatcgtggtcatcatcgccgtctggctgctgctgaccgaactcatgaccaccaaggtgctgctgttcttcaccagccaactgctcgtggccgtgtttgtgtttggaaatacatgtaaaacaatctttgaggctatcatcttcgtgttcgtgatgcaccctttcgacgtgggcgatagatgtgtgatcgatggaaccaccatgctggtcgaggagatgaatatcctcaccacagtgtttctgaagtgggacaaagaaaaagtgtactaccccaacgccgtgctgagcaccaaagctattggaaattactataggtcccccgaccaagtggactctctggagtttagcatcgattttagaacccctctgtccaaaatcggagagattaaggaaaggatcaaaaaatatctgcaccagaatccccatctgtggcaccctaatcacaatctggtggtcaaggagatcgagaatgtgaacaagatcaaaacacaactcattttcaatcacaccatgaacttccaagagttccccgagaggatgaagagaaggacagaactcgtgctggagctgaagaaaatcttcgaggagctggacatcaaatacaatctgctgccccaagaagtcattctgaacaacgtgggaccctga

SEQ ID NO: 15 - DNA, 27 bp, synthetic construct
  misc_feature 1..27: note = Description of artificial sequence: synthetic oligonucleotide sequence
  source 1..27: mol_type = other DNA
ccagtgtcaccttatagggaagaagcg

SEQ ID NO: 16 - DNA, 22 bp, synthetic construct
  misc_feature 1..22: note = Description of artificial sequence: synthetic oligonucleotide sequence
  source 1..22: mol_type = other DNA
ccctcgacgtagttcccctagc

SEQ ID NO: 17 - DNA, 40 bp, synthetic construct
  misc_feature 1..40: note = Description of artificial sequence: synthetic oligonucleotide sequence
  source 1..40: mol_type = other DNA
cacatcgattcacatattgcggatactaatttcttggggc

SEQ ID NO: 18 - DNA, 37 bp, synthetic construct
  misc_feature 1..37: note = Description of artificial sequence: synthetic oligonucleotide sequence
  source 1..37: mol_type = other DNA
acacccggggctagctttcatccaccgaataaacacc

SEQ ID NO: 19 - DNA, 36 bp, synthetic construct
  misc_feature 1..36: note = Description of artificial sequence: synthetic oligonucleotide sequence
  source 1..36: mol_type = other DNA
cacatcgatacatcattgaccagaagcaaggcactc

SEQ ID NO: 20 - DNA, 33 bp, synthetic construct
  misc_feature 1..33: note = Description of artificial sequence: synthetic oligonucleotide sequence
  source 1..33: mol_type = other DNA
tggcatcttcattccatttgaataggttctttg

SEQ ID NO: 21 - DNA, 24 bp, synthetic construct
  misc_feature 1..24: note = Description of artificial sequence: synthetic oligonucleotide sequence
  source 1..24: mol_type = other DNA
gcatacccgacatggtcgacaagc

SEQ ID NO: 22 - DNA, 30 bp, synthetic construct
  misc_feature 1..30: note = Description of artificial sequence: synthetic oligonucleotide sequence
  source 1..30: mol_type = other DNA
tgacaagtttgtttaactgcttcactgctg

SEQ ID NO: 23 - DNA, 34 bp, synthetic construct
  misc_feature 1..34: note = Description of artificial sequence: synthetic oligonucleotide sequence
  source 1..34: mol_type = other DNA
aggtctttagattaactcttcaacatgggtaagg

SEQ ID NO: 24 - DNA, 31 bp, synthetic construct
  misc_feature 1..31: note = Description of artificial sequence: synthetic oligonucleotide sequence
  source 1..31: mol_type = other DNA
acaagacttcattttgcacccttctttatcg

SEQ ID NO: 25 - DNA, 27 bp, synthetic construct
  misc_feature 1..27: note = Description of artificial sequence: synthetic oligonucleotide sequence
  source 1..27: mol_type = other DNA
gattgagcaaacaaaaggcgcatgaag

SEQ ID NO: 26 - DNA, 27 bp, synthetic construct
  misc_feature 1..27: note = Description of artificial sequence: synthetic oligonucleotide sequence
  source 1..27: mol_type = other DNA
aaattttgacctacgttgaccgtcagc

SEQ ID NO: 27 - DNA, 24 bp, synthetic construct
  misc_feature 1..24: note = Description of artificial sequence: synthetic oligonucleotide sequence
  source 1..24: mol_type = other DNA
tgatcaggctgcgcttaaacatgc

SEQ ID NO: 28 - DNA, 34 bp, synthetic construct
  misc_feature 1..34: note = Description of artificial sequence: synthetic oligonucleotide sequence
  source 1..34: mol_type = other DNA
acattctactttgtttgcaattgttttcccactc

SEQ ID NO: 29 - DNA, 26 bp, synthetic construct
  misc_feature 1..26: note = Description of artificial sequence: synthetic oligonucleotide sequence
  source 1..26: mol_type = other DNA
aagaaacattaagctgcacctgctcc

SEQ ID NO: 30 - DNA, 24 bp, synthetic construct
  misc_feature 1..24: note = Description of artificial sequence: synthetic oligonucleotide sequence
  source 1..24: mol_type = other DNA
gagaggtccaccaaccttgactgg

SEQ ID NO: 31 - DNA, 22 bp, synthetic construct
  misc_feature 1..22: note = Description of artificial sequence: synthetic oligonucleotide sequence
  source 1..22: mol_type = other DNA
agcaacggtctgacgcatgtcc

SEQ ID NO: 32 - DNA, 23 bp, synthetic construct
  misc_feature 1..23: note = Description of artificial sequence: synthetic oligonucleotide sequence
  source 1..23: mol_type = other DNA
gtgccagtgggaagagttgagac

SEQ ID NO: 33 - DNA, 25 bp, synthetic construct
  misc_feature 1..25: note = Description of artificial sequence: synthetic oligonucleotide sequence
  source 1..25: mol_type = other DNA
cagagaaagtctcgacaaccatggg

SEQ ID NO: 34 - DNA, 31 bp, synthetic construct
  misc_feature 1..31: note = Description of artificial sequence: synthetic oligonucleotide sequence
  source 1..31: mol_type = other DNA
caaatcattgaaaacgtgaagggaagcactg

SEQ ID NO: 35 - DNA, 30 bp, synthetic construct
  misc_feature 1..30: note = Description of artificial sequence: synthetic oligonucleotide sequence
  source 1..30: mol_type = other DNA
cctatgagatacttagcctgttagccatgc

SEQ ID NO: 36 - DNA, 28 bp, synthetic construct
  misc_feature 1..28: note = Description of artificial sequence: synthetic oligonucleotide sequence
  source 1..28: mol_type = other DNA
gcccttctatagtagtctcacctcttcg

SEQ ID NO: 37 - DNA, 24 bp, synthetic construct
  misc_feature 1..24: note = Description of artificial sequence: synthetic oligonucleotide sequence
  source 1..24: mol_type = other DNA
gccatgcgcagcatatgtactagc

SEQ ID NO: 38 - DNA, 33 bp, synthetic construct
  misc_feature 1..33: note = Description of artificial sequence: synthetic oligonucleotide sequence
  source 1..33: mol_type = other DNA
gctatttcttattctcctgagcacaacatactg

SEQ ID NO: 39 - DNA, 28 bp, synthetic construct
  misc_feature 1..28: note = Description of artificial sequence: synthetic oligonucleotide sequence
  source 1..28: mol_type = other DNA
tgatcgctgtgtcgtagatggaacaatg

SEQ ID NO: 40 - DNA, 31 bp, synthetic construct
  misc_feature 1..31: note = Description of artificial sequence: synthetic oligonucleotide sequence
  source 1..31: mol_type = other DNA
ctaatggattgcaaactaggagatgcttagc

SEQ ID NO: 41 - protein, 13 aa, synthetic construct
  REGION 1..13: note = Description of artificial sequence: synthetic peptide sequence
WRYYESSLEPYPD

SEQ ID NO: 42 - protein, 752 aa, synthetic construct
  REGION 1..752: note = Description of artificial sequence: synthetic polypeptide sequence
MGSYLHEPPGDEPSMRIEQPKTADRAPEQVAIHICEPSKVVTESFPFSETAEPEAKSKNCPCPEIARIGPCPNKPPKIPINRGLSRISTNKSRPKSRFGEPSWPVESSLDLTSQSPVSPYREEAFSVENCGTAGSRRGSFARGTTSRAASSSRKDETKEGPDEKEVYQRVTAQLSARNQKRMTVKLMIELSVFLCLLGCLVCSLTVDGFKRYTVIGLDIWKWFLLLLVIFSGMLITHWIVHVAVFFVEWKFLMRKNVLYFTHGLKTSVEVFIWITVVLATWVMLIKPDVNQPHQTRKILEFVTWTIVTVLIGAFLWLVKTTLLKILASSFHLNEFFDRIQESVFHHSVLQTLAGRPVVELAQGISRTESQDGAGQVSFMEHTKTQNKKVVDVGKLHQMKQEKVPAWTMQLLVDVVSNSGLSTMSGMLDEDMVEGGVELDDDEITNEEQAIATAVRIFDNIVQDKVDQSYIDRVDLHRFLIWEEVDHLFPLFEVNEKGQISLKAFAKWVVKVYNDQAALKHALNDNKTAVKQLNKLVTAILIVMMIVIWLIVTGIATTKLIVLLSSQLVVAAFIFGNTCKTIFEAIIFVFVMHPFDVGDRCVIDGNKMLVEEMNILTTVFLKWDKEKVYYPNSILCTKAIGNFFRSPDQGDVLEFSVDFTTPVLKIGDLKDRIKMYLEQNLNFWHPQHNMVVKEIENVNKIKMALFVNHTINFQDFAEKNRRRSELVLELKKIFEELDIKYNLLPQEISIRNM

(19) United States
(12) Patent Application Publication
(10) Pub. No.: US 2023/0340534 A1
(43) Pub. Date: Oct. 26, 2023

(54) SONOGENETIC STIMULATION OF CELLS EXPRESSING A HETEROLOGOUS MECHANOSENSITIVE PROTEIN

(71) Applicants: Salk Institute for Biological Studies, La Jolla, CA (US); The Scripps Research Institute, La Jolla, CA (US)

(72) Inventors: Sreekanth CHALASANI, La Jolla, CA (US); Carl PROCKO, La Jolla, CA (US); Joanne CHORY, La Jolla, CA (US); Jose Mendoza LOPEZ, La Jolla, CA (US); Marc Duque RAMIREZ, La Jolla, CA (US); Corinne LEE-KUBLI, La Jolla, CA (US); Swetha MURTHY, La Jolla, CA (US); Ardem PATAPOUTIAN, La Jolla, CA (US); Seyed Ali Reza MOUSAVI, La Jolla, CA (US); William T. KEENAN, La Jolla, CA (US); Yusuf TUFAIL, San Diego, CA (US); Janki PATEL, La Jolla, CA (US)

(73) Assignees: Salk Institute for Biological Studies, La Jolla, CA (US); The Scripps Research Institute, La Jolla, CA (US)

(21) Appl. No.: 18/315,383

(22) Filed: May 10, 2023

Related U.S. Application Data
(63) Continuation of application No. PCT/US2021/058708, filed on Nov. 10, 2021.
(60) Provisional application No. 63/112,256, filed on Nov. 11, 2020.

(51) Int. Cl. C12N 15/86; C12N 13/00; C07K 14/47

(52) U.S. Cl. CPC C12N 15/86; C12N 13/00; C07K 14/4702; C12N 2750/14143

(57) ABSTRACT

Provided and described are mechanosensory polypeptides and encoding polynucleotide products and compositions thereof, methods of expressing such polypeptides and polynucleotides in a cell type of interest, and methods of inducing and/or modifying the activity and/or function of various types of cells that express the exogenous mechanosensory polypeptides using ultrasound.

### CROSS-REFERENCE TO RELATED APPLICATIONS

This application is a continuation under 35 U.S.C. § 111(a) of PCT International Patent Application No. PCT/US2021/058708, filed Nov. 10, 2021, designating the United States and published in English, which claims priority to and benefit of U.S. Provisional Patent Application No. 63/112,256, filed Nov. 11, 2020, the entire contents of each of which are incorporated by reference herein.

### STATEMENT OF RIGHTS TO INVENTIONS MADE UNDER FEDERALLY SPONSORED RESEARCH

This invention was made with government support under Grant Nos. MH111534, NS115591, 5R35GM122604, HL143297, P30 014195, CA014195, NS072031, and F32GM101876 awarded by the National Institutes of Health and by Grant Nos. DBI-0735191, DBI-1265383, and DBI-1743442 awarded by the National Science Foundation. The government has certain rights in the invention.

### SEQUENCE LISTING

The present application contains a Sequence Listing which has been submitted electronically in XML format following conversion from the originally filed TXT format.

The content of the electronic XML Sequence_Listing, (Date of creation: May 4, 2023; Size: 68,628 bytes; Name: 167776-011602US-Sequence_Listing.xml), and the original TXT format, is herein incorporated by reference in its entirety.

### BACKGROUND

Understanding how neural circuits generate specific behaviors requires an ability to identify the participating neurons, record and perturb their activity patterns. The best-understood motor circuit, the crab stomatogastric ganglion (STG) has benefited from electrophysiological access to well-defined cell types as well as an ability to manipulate them. A number of approaches have been developed for manipulating neuronal activity using light (optogenetics) or small molecules. While these methods have revealed insights into circuit computations in a number of model systems including mice, they are associated with drawbacks, such as the difficulty of delivering a stimulus to the target neurons present in deeper brain regions. Thus, there remains a need for new products and methods for the non-invasive manipulation of target neurons and other cell types.

### SUMMARY

Provided and featured herein are products and compositions featuring mechanosensory polypeptides and polynucleotides, methods for expressing such polypeptides and polynucleotides in a cell type of interest, and methods for inducing the activation of the mechanosensory polypeptide in neurons and other cell types using ultrasound.

In one aspect, a method for inducing cation or anion influx or efflux in a cell is provided, in which the method involves expressing in the cell a heterologous, mechanosensory polypeptide selected from one or more of the following: DmFLYC1, DmFLYC2, DcFLYC1.1, DcFLYC1.2, and DmOSCA; and applying ultrasound to the cell, thereby inducing cation or anion influx or efflux in the cell.

In another aspect, a method for initiating a cellular response to mechanical deformation or stretch caused by ultrasound is provided, in which the method involves transducing a cell to express a heterologous, mechanosensory polypeptide selected from one or more of the following: DmFLYC1, DmFLYC2, DcFLYC1.1, DcFLYC1.2, and DmOSCA; applying ultrasound to the cell; and inducing cation or anion influx or efflux in the mechanosensory polypeptide expressing cell and an alteration in cell activity and/or function following the application of ultrasound, thereby initiating a cellular response to mechanical deformation or stretch caused by ultrasound.

In another aspect, a method for inducing a cellular response to mechanical deformation or stretch caused by ultrasound and modulating activity and/or function of a cell is provided, in which the method involves transducing a cell to express a heterologous, mechanosensory polypeptide selected from one or more of the following: DmFLYC1, DmFLYC2, DcFLYC1.1, DcFLYC1.2, and DmOSCA; applying ultrasound to the mechanosensory polypeptide-expressing cell; and inducing cation or anion influx or efflux in the mechanosensory polypeptide-expressing cell and an alteration in cell activity and/or function following the application of ultrasound, thereby inducing a cellular response to mechanical deformation or stretch caused by ultrasound and modulating cell activity and/or function.

In an embodiment of the above-delineated methods, the heterologous, mechanosensory polypeptide is a variant of the DmFLYC1 polypeptide. In an embodiment, the variant is an R334E FLYC1 variant polypeptide. In embodiments, the R334E FLYC1 variant polypeptide comprises an amino acid sequence having at least 85% or at least 95% sequence identity to the sequence of SEQ ID NO: 42. In an embodiment, the R334E FLYC1 variant polypeptide comprises or consists essentially of the sequence of SEQ ID NO: 42. In an embodiment of any of the above aspects, the cell is sensitized to mechanical deformation or stretch caused by ultrasound. In an embodiment of any of the above aspects, the application of ultrasound effects a change in mechanosensory polypeptide conductance in the cell and modulates a cell activity and/or function.

In an embodiment of any of the above aspects and embodiments thereof, applying ultrasound induces an anion influx or efflux in the cell. In some embodiments, applying the ultrasound induces an anion influx that inhibits or silences an activity and/or function of the cell. In some embodiments, applying the ultrasound induces an anion efflux that excites or stimulates an activity and/or function of a plant cell. In some embodiments, the polypeptide is selected from one or more of the following: DmFLYC1, DmFLYC2, DcFLYC1.1, and DcFLYC1.2. In some embodiments, the polypeptide contains a sequence having at least 85% sequence identity to a polypeptide sequence selected from SEQ ID NOs: 5, 7, 11, 13, or 42. In some embodiments, the polypeptide is encoded by a sequence having at least 85% sequence identity to a polynucleotide sequence selected from SEQ ID NOs: 6, 8, 12, or 14. In some embodiments, the polypeptide is encoded by a sequence having at least 95% sequence identity to a polynucleotide sequence selected from SEQ ID NOs: 6, 8, 12, or 14. In some embodiments, the polypeptide is encoded by a sequence comprising or consisting essentially of a polynucleotide sequence selected from SEQ ID NOs: 6, 8, 12, or 14.

In an embodiment of any of the above-delineated aspects and embodiments thereof, applying ultrasound induces a cation influx in the cell. In some embodiments, the cation influx increases activity of the cell. In some embodiments, the polypeptide is DmOSCA. In some embodiments, the polypeptide contains a sequence having at least 85% sequence identity to SEQ ID NO: 9. In some embodiments, the polypeptide contains a sequence having at least 95% sequence identity to SEQ ID NO: 9. In some embodiments, the polypeptide comprises or consists essentially of the sequence of SEQ ID NO: 9. In some embodiments, the polypeptide is encoded by a polynucleotide sequence having at least 85% sequence identity to SEQ ID NO: 10. In some embodiments, the polypeptide is encoded by a polynucleotide sequence having at least 95% sequence identity to SEQ ID NO: 10. In some embodiments, the polypeptide is encoded by a polynucleotide sequence comprising or consisting essentially of SEQ ID NO: 10.

In an embodiment of any of the above-delineated aspects and embodiments thereof, the polypeptide is encoded by a polynucleotide sequence codon-optimized for expression in a mammalian or human cell and is non-naturally occurring. In an embodiment of any of the above-delineated aspects and embodiments thereof, the polypeptide is expressed in the cell following transduction of the cell by a plasmid or viral vector containing a polynucleotide sequence encoding the polypeptide. In an embodiment of any of the above-delineated aspects and embodiments thereof, the cell is transduced by a viral vector selected from a lentivirus vector or an adeno-associated virus (AAV) vector. In an embodiment of any of the above-delineated aspects and embodiments thereof, the cell is a mammalian cell. In an embodiment of any of the above-delineated aspects and embodiments thereof, the cell is a human cell. In an embodiment of any of the above-delineated aspects and embodiments thereof, the cell is one or more of a muscle cell, a cardiac muscle cell, an insulin secreting cell, a pancreatic cell, a kidney cell, or a neuronal cell. In an embodiment, the cell is a neuronal cell. In an embodiment, the cell is a plant cell and anion efflux and/or stimulation of the plant cell activity or function is induced by ultrasound application to the cell. In an embodiment, the cell is in vitro, ex vivo, or in vivo.

In an embodiment of any of the above-delineated aspects and embodiments thereof, the ultrasound has a frequency of about 0.2 MHz to about 20 MHz. In an embodiment of any of the above-delineated aspects and embodiments thereof, the ultrasound has a focal zone of about 1 cubic millimeter to about 1 cubic centimeter. In an embodiment, the methods of any of the above aspects can further involve contacting the cell with a microbubble prior to applying ultrasound.

In an embodiment of any of the above-delineated aspects and embodiments thereof, the cell is in a subject. In an embodiment, the subject is a mammal. In an embodiment, the subject is a human.

In an embodiment of any of the above-delineated aspects and embodiments thereof, the ultrasound is generated using an opto-acoustic system or a transducer. In an embodiment of any of the above-delineated aspects and embodiments thereof, the ultrasound is generated using a lead zirconate titanate (PZT) transducer.

In another aspect is provided a plasmid or viral vector containing a polynucleotide encoding a mechanosensory polypeptide selected from one or more of the following: DmFLYC1, DmFLYC2, DcFLYC1.1, DcFLYC1.2, DmOSCA, or a variant thereof. In an embodiment, the mechanosensory polypeptide is a variant of the DmFLYC1 polypeptide. In an embodiment, the variant is an R334E FLYC1 variant polypeptide. In embodiments, the R334E FLYC1 variant polypeptide comprises an amino acid sequence having at least 85% or at least 95% sequence identity to the sequence of SEQ ID NO: 42. In an embodiment, the R334E FLYC1 variant polypeptide comprises or consists essentially of the sequence of SEQ ID NO: 42.

In another aspect is provided a cell containing the plasmid or viral vector of the above-delineated aspect or a heterologous gene sequence encoding a polypeptide selected from one or more of the following: DmFLYC1, DmFLYC2, DcFLYC1.1, DcFLYC1.2, and DmOSCA, or a variant thereof. In an embodiment, the encoded polypeptide is a variant of the DmFLYC1 polypeptide. In an embodiment, the variant is an R334E FLYC1 variant polypeptide. In embodiments, the R334E FLYC1 variant polypeptide comprises an amino acid sequence having at least 85% or at least 95% sequence identity to the sequence of SEQ ID NO: 42. In an embodiment, the R334E FLYC1 variant polypeptide comprises or consists essentially of the sequence of SEQ ID NO: 42.

In an embodiment, the vector is a viral vector, which is a lentivirus vector or an adeno-associated virus (AAV) vector.

In an embodiment of any of the above-delineated aspects and embodiments thereof, the polypeptide comprises a sequence having at least 85% sequence identity to a polypeptide sequence selected from one or more of SEQ ID NOs: 5, 7, 11, 13, or 9. In an embodiment of any of the above-delineated aspects and embodiments thereof, the polypeptide comprises a sequence having at least 95% sequence identity to a polypeptide sequence selected from one or more of SEQ ID NOs: 5, 7, 11, 13, or 9. In an embodiment of any of the above-delineated aspects and embodiments thereof, the polypeptide comprises a sequence comprising or consisting essentially of a sequence selected from one or more of SEQ ID NOs: 5, 7, 11, 13, or 9. In an embodiment of any of the above-delineated aspects and embodiments thereof, the polypeptide is encoded by a sequence having at least 85% sequence identity to a polynucleotide sequence selected from one or more of SEQ ID NOs: 6, 8, 12, 14, or 10. In an embodiment of any of the above-delineated aspects and embodiments thereof, the polypeptide is encoded by a sequence having at least 95% sequence identity to a polynucleotide sequence selected from one or more of SEQ ID NOs: 6, 8, 12, 14, or 10. In an embodiment of any of the above-delineated aspects and embodiments thereof, the polypeptide is encoded by a sequence comprising or consisting essentially of a polynucleotide sequence selected from one or more of SEQ ID NOs: 6, 8, 12, 14, or 10.

In an embodiment of any of the above-delineated aspects and embodiments thereof, the cell is a plant cell and anion efflux and/or stimulation of the plant cell activity or function is induced by ultrasound application to the cell. In an embodiment of any of the above-delineated aspects and embodiments thereof, the cell is a mammalian cell. In an embodiment of any of the above-delineated aspects and embodiments thereof, the cell is a human cell. In an embodiment of any of the above-delineated aspects and embodiments thereof, the cell is one or more of a muscle cell, a cardiac muscle cell, an insulin secreting cell, a pancreatic cell, a kidney cell, or a neuronal cell. In an embodiment, the cell is a neuronal cell.

In another aspect, an isolated polynucleotide encoding a mechanosensory DmFLYC1 polypeptide, or a variant thereof, is provided. In another aspect, an isolated polynucleotide encoding a mechanosensory DmFLYC2 polypeptide is provided. In another aspect, an isolated polynucleotide encoding a mechanosensory DcFLYC1.1 polypeptide is provided. In another aspect, an isolated polynucleotide encoding a mechanosensory DcFLYC1.2 polypeptide is provided. In another aspect, an isolated polynucleotide encoding a mechanosensory DmOSCA polypeptide is provided.

In a further aspect, a mechanosensory polypeptide encoded by the polynucleotide of any one of the above-delineated aspects and embodiments thereof is provided. In another aspect, a plasmid or viral vector containing the isolated polynucleotide of any one of the above-delineated aspects and embodiments thereof is provided.

In an aspect, a cell containing the isolated polynucleotide of any one of the above-delineated aspects and embodiments thereof is provided. In an aspect, a cell expressing the mechanosensory polypeptide of any one of the above-delineated aspects and embodiments thereof is provided.

In another aspect, a composition comprising the cell of any of above-delineated aspects and embodiments thereof is provided. In an embodiment, the composition further comprises a pharmaceutically acceptable carrier, excipient, or diluent.

In an embodiment of any of the above-delineated aspects and embodiments thereof, the isolated polynucleotide is codon-optimized for expression in mammalian cells. In an embodiment of any of the above-delineated aspects and embodiments thereof, the isolated polynucleotide is codon-optimized for expression in human cells.

In an embodiment of any of the above-delineated aspects and embodiments thereof, the polypeptide is DmFLYC1, which contains a sequence having at least 85% sequence identity to SEQ ID NO: 5 or to SEQ ID NO: 42. In an embodiment of any of the above-delineated aspects and embodiments thereof, the polypeptide is DmFLYC1, comprising a sequence having at least 95% sequence identity to SEQ ID NO: 5 or to SEQ ID NO: 42. In an embodiment of any of the above-delineated aspects and embodiments thereof, the polypeptide is DmFLYC1 comprising or consisting essentially of SEQ ID NO: 5 or SEQ ID NO: 42. In an embodiment of any of the above-delineated aspects and embodiments thereof, the polypeptide is encoded by a polynucleotide sequence having at least 85% sequence identity to SEQ ID NO: 6. In an embodiment of any of the above-delineated aspects and embodiments thereof, the polypeptide is encoded by a polynucleotide sequence having at least 95% sequence identity to SEQ ID NO: 6. In an embodiment of any of the above-delineated aspects and embodiments thereof, the polypeptide is encoded by a polynucleotide sequence comprising or consisting essentially of SEQ ID NO: 6.

In an embodiment of any of the above-delineated aspects and embodiments thereof, the polypeptide is DmFLYC2 and contains a sequence having at least 85% sequence identity or at least 95% sequence identity to SEQ ID NO: 7. In an embodiment of any of the above-delineated aspects and embodiments thereof, the polypeptide is DmFLYC2 comprising or consisting essentially of SEQ ID NO: 7. In an embodiment of any of the above-delineated aspects and embodiments thereof, the polypeptide is encoded by a polynucleotide sequence having at least 85% or at least 95% sequence identity to SEQ ID NO: 8. In an embodiment of any of the above-delineated aspects and embodiments thereof, the polypeptide is encoded by a polynucleotide sequence comprising or consisting essentially of SEQ ID NO: 8.

In an embodiment of any of the above-delineated aspects and embodiments thereof, the polypeptide is DcFLYC1.1 and contains a sequence having at least 85% or at least 95% sequence identity to SEQ ID NO: 11. In an embodiment of any of the above-delineated aspects and embodiments thereof, the polypeptide is DcFLYC1.1 comprising or consisting essentially of SEQ ID NO: 11. In an embodiment of any of the above-delineated aspects and embodiments thereof, the polypeptide is encoded by a polynucleotide sequence having at least 85% or at least 95% sequence identity to SEQ ID NO: 12. In an embodiment of any of the above-delineated aspects and embodiments thereof, the polypeptide is encoded by a polynucleotide sequence comprising or consisting essentially of SEQ ID NO: 12

In an embodiment of any of the above-delineated aspects and embodiments thereof, the polypeptide is DcFLYC1.2 and contains a sequence having at least 85% or at least 95% sequence identity to SEQ ID NO: 13. In an embodiment of any of the above-delineated aspects and embodiments thereof, the polypeptide is DcFLYC1.2 comprising or consisting essentially of SEQ ID NO: 13. In an embodiment of any of the above-delineated aspects and embodiments thereof, the polypeptide is encoded by a polynucleotide sequence having at least 85% or at least 95% sequence identity to SEQ ID NO: 14. In an embodiment of any of the above-delineated aspects and embodiments thereof, the polypeptide is encoded by a polynucleotide sequence comprising or consisting essentially of SEQ ID NO: 14.

In an embodiment of any of the above-delineated aspects and embodiments thereof, the polypeptide is DmOSCA and contains a sequence having at least 85% or at least 95% sequence identity to SEQ ID NO: 9. In an embodiment of any of the above-delineated aspects and embodiments thereof, the polypeptide is DmOSCA comprising or consisting essentially of SEQ ID NO: 9. In an embodiment of any of the above-delineated aspects and embodiments thereof, the polypeptide is encoded by a polynucleotide sequence having at least 85% or at least 95% sequence identity to SEQ ID NO: 10. In an embodiment of any of the above-delineated aspects and embodiments thereof, the polypeptide is encoded by a polynucleotide sequence comprising or consisting essentially of SEQ ID NO: 10.

In an embodiment of any of the above-delineated aspects and embodiments thereof, the polypeptide is DmFLYC1. In an embodiment of any of the above-delineated aspects and embodiments thereof, the DmFLYC1 polypeptide is encoded by a polynucleotide sequence comprising or consisting essentially of SEQ ID NO: 5 or comprising or consisting essentially of SEQ ID NO: 42.

In an embodiment of any one of the above-delineated aspects and embodiments thereof, the anion influx or efflux is a chloride anion influx or efflux.

Compositions and articles defined by the aspects and embodiments as described herein were isolated or otherwise manufactured in connection with the examples provided herein. Other features and advantages of the aspects and embodiments provided herein will be apparent from the detailed description, and from the claims.

### Definitions

Unless defined otherwise, technical and scientific terms used herein have the same meaning as commonly understood by a person of ordinary skill in the art. See, e.g., Singleton et al., DICTIONARY OF MICROBIOLOGY AND MOLECULAR BIOLOGY 2nd ed., J. Wiley & Sons (New York, NY 1994); Sambrook et al., MOLECULAR CLONING, A LABORATORY MANUAL, Cold Spring Harbor Press (Cold Spring Harbor, N Y 1989). Any methods, devices and materials similar or equivalent to those described herein can be used in the practice of the aspects and embodiments described herein. The following definitions are provided to facilitate understanding of certain terms used frequently herein and are not meant to limit the scope of the present disclosure.

By “FLYC1” or “DmFLYC1” is meant a mechanosensory polypeptide capable of conferring ultrasound sensitivity on a cell, e.g., a neuron, and having at least about 85% amino acid sequence identity to the DmFLYC1 polypeptide sequence provided below, a fragment thereof, or a human ortholog thereof, and having the biological activity described herein. In embodiments, the mechanosensory polypeptide has at least about 90%, at least about 95%, or at least about 98% amino acid sequence identity to the DmFLYC1 polypeptide sequence provided below, a fragment thereof, or a human ortholog thereof, and has the biological activity described herein. In some embodiments, the DmFLYC1 polypeptide is substantially identical to the DmFLYC1 polypeptide sequence provided below or a functional variant, isoform, homolog, or ortholog having substantial identity thereto. In some embodiments, the DmFLYC1 polypeptide is a functional homolog, isoform, ortholog, or fragment of the DmFLYC1 polypeptide sequence provided below. In some embodiments, the FLYC1 polypeptide heterologously expressed in cells, such as, without limitation, fibroblasts and insulin-secreting (INS) cells, is inhibitory to the cells upon ultrasound (US) stimulation. In some embodiments, the DmFLYC1 polypeptide is or includes the DmFLYC1 polypeptide sequence provided immediately below.

DmFLYC1 polypeptide sequence:
(SEQ ID NO: 5)
MGSYLHEPPGDEPSMRIEQPKTADRAPEQVAIHICEPSKVVTESFPFSET
AEPEAKSKNCPCPEIARIGPCPNKPPKIPINRGLSRISTNKSRPKSRFGE
PSWPVESSLDLTSQSPVSPYREEAFSVENCGTAGSRRGSFARGTTSRAAS
SSRKDETKEGPDEKEVYQRVTAQLSARNQKRMTVKLMIELSVFLCLLGCL
VCSLTVDGFKRYTVIGLDIWKWFLLLLVIFSGMLITHWIVHVAVFFVEWK
FLMRKNVLYFTHGLKTSVEVFIWITVVLATWVMLIKPDVNQPHQTRKILE
FVTWTIVTVLIGAFLWLVKTTLLKILASSFHLNRFFDRIQESVFHHSVLQ
TLAGRPVVELAQGISRTESQDGAGQVSFMEHTKTQNKKVVDVGKLHQMKQ
EKVPAWTMQLLVDVVSNSGLSTMSGMLDEDMVEGGVELDDDEITNEEQAI
ATAVRIFDNIVQDKVDQSYIDRVDLHRFLIWEEVDHLFPLFEVNEKGQIS
LKAFAKWVVKVYNDQAALKHALNDNKTAVKQLNKLVTAILIVMMIVIWLI
VTGIATTKLIVLLSSQLVVAAFIFGNTCKTIFEAIIFVFVMHPFDVGDRC
VIDGNKMLVEEMNILTTVFLKWDKEKVYYPNSILCTKAIGNFFRSPDQGD
VLEFSVDFTTPVLKIGDLKDRIKMYLEQNLNFWHPQHNMVVKEIENVNKI
KMALFVNHTINFQDFAEKNRRRSELVLELKKIFEELDIKYNLLPQEISIR
NM.

In an embodiment, a variant of the FLYC1 polypeptide is provided in which the amino acid arginine (R) at position 334 is replaced with the amino acid glutamic acid (E), i.e., “R334E variant”. The “R” amino acid that is changed to “E” at position 334 in the FLYC1 amino acid sequence is underlined and in bold in the DmFLYC1 polypeptide sequence presented supra. The amino acid sequence of the FLYC1 R334E polypeptide variant containing an “E” at position 334 is shown below. The variant “E” at position 334 is designated in bold and underlining in the below

sequence.

(SEQ ID NO: 42)
MGSYLHEPPGDEPSMRIEQPKTADRAPEQVAIHICEPSKVVTESFPFSET
AEPEAKSKNCPCPEIARIGPCPNKPPKIPINRGLSRISTNKSRPKSRFGE
PSWPVESSLDLTSQSPVSPYREEAFSVENCGTAGSRRGSFARGTTSRAAS
SSRKDETKEGPDEKEVYQRVTAQLSARNQKRMTVKLMIELSVFLCLLGCL
VCSLTVDGFKRYTVIGLDIWKWFLLLLVIFSGMLITHWIVHVAVFFVEWK
FLMRKNVLYFTHGLKTSVEVFIWITVVLATWVMLIKPDVNQPHQTRKILE
FVTWTIVTVLIGAFLWLVKTTLLKILASSFHLNEFFDRIQESVFHHSVLQ
TLAGRPVVELAQGISRTESQDGAGQVSFMEHTKTQNKKVVDVGKLHQMKQ
EKVPAWTMQLLVDVVSNSGLSTMSGMLDEDMVEGGVELDDDEITNEEQAI
ATAVRIFDNIVQDKVDQSYIDRVDLHRFLIWEEVDHLFPLFEVNEKGQIS
LKAFAKWVVKVYNDQAALKHALNDNKTAVKQLNKLVTAILIVMMIVIWLI
VTGIATTKLIVLLSSQLVVAAFIFGNTCKTIFEAIIFVFVMHPFDVGDRC
VIDGNKMLVEEMNILTTVFLKWDKEKVYYPNSILCTKAIGNFFRSPDQGD
VLEFSVDFTTPVLKIGDLKDRIKMYLEQNLNFWHPQHNMVVKEIENVNKI
KMALFVNHTINFQDFAEKNRRRSELVLELKKIFEELDIKYNLLPQEISIR
NM

By “DmFLYC1 polynucleotide” or “FLYC1 polynucleotide” is meant a nucleic acid molecule encoding a DmFLYC1 polypeptide. In particular embodiments, the codons of the DmFLYC1 polynucleotide are optimized for expression in an organism of interest or in the cells of an organism of interest (e.g., optimized for human expression or expression in human cells, mammalian expression or mammalian cell expression, plant expression or plant cell expression). The sequence of an exemplary DmFLYC1 polynucleotide is provided immediately below. In some embodiments, the DmFLYC1 nucleic acid molecule is substantially identical to the DmFLYC1 nucleic acid molecule provided below or a functional variant, ortholog, or homolog having substantial identity thereto. In some embodiments, the DmFLYC1 nucleic acid molecule is a nucleic acid molecule with the DmFLYC1 polynucleotide sequence provided below. In some embodiments, the DmFLYC1 nucleic acid molecule is a functional homolog, isoform, or fragment of the nucleic acid molecule with the sequence provided below. In some embodiments, the DmFLYC1 nucleic acid molecule is or includes the DmFLYC1 polynucleotide sequence provided immediately below. In some embodiments, for example, for expression in a mammalian cell, e.g., a human cell, the codon-optimized DmFLYC1 polynucleotide sequence provided below is used, or a sequence with at least 85% sequence identity thereto is used. In embodiments, a sequence with at least 90%, at least 95%, or at least 98% sequence identity thereto is used.

DmFLYC1 polynucleotide sequence (DmFLYCI
codon-optimized polynucleotide sequence):
(SEQ ID NO: 6)
ATGGGATCCTATTTACATGAGCCCCCCGGCGACGAGCCTTCCATGAGGAT
CGAGCAGCCTAAGACAGCTGACAGAGCTCCCGAGCAAGTTGCCATTCACA
TCTGTGAACCTTCCAAAGTCGTGACCGAGTCCTTTCCCTTCTCCGAGACA
GCCGAGCCCGAGGCTAAGTCCAAGAACTGCCCTTGCCCCGAAATTGCCAG
AATTGGCCCTTGCCCTAACAAACCTCCCAAGATCCCTATCAATAGGGGTT
TATCTCGTATCTCCACCAACAAGAGCAGACCTAAGTCCAGATTCGGAGAG
CCCAGCTGGCCCGTTGAGAGCTCTTTAGACCTCACAAGCCAGTCCCCCGT
CAGCCCTTATCGTGAGGAAGCCTTCAGCGTCGAAAATTGCGGCACAGCCG
GCTCTCGTAGGGGATCCTTCGCTAGGGGAACAACCAGCAGAGCCGCCTCC
AGCTCTCGTAAGGATGAAACAAAGGAGGGCCCCGATGAAAAGGAAGTGTA
CCAGAGGGTGACAGCCCAGCTGAGCGCTAGAAACCAGAAGAGGATGACCG
TCAAGCTGATGATCGAACTGTCCGTGTTTTTATGTCTGCTGGGCTGTCTG
GTCTGCTCTTTAACAGTGGATGGATTCAAGAGGTACACCGTGATCGGTTT
AGACATCTGGAAATGGTTTTTACTGCTGCTGGTCATCTTCAGCGGAATGC
TGATCACACACTGGATCGTGCACGTCGCCGTCTTCTTCGTGGAGTGGAAA
TTTCTGATGAGAAAAAACGTGCTGTACTTTACCCACGGTTTAAAAACCTC
CGTCGAGGTGTTCATCTGGATCACAGTCGTGCTCGCCACTTGGGTGATGC
TGATTAAACCCGACGTGAACCAGCCCCACCAAACTCGTAAGATTTTAGAG
TTTGTGACTTGGACCATCGTGACAGTCCTCATTGGCGCCTTTTTATGGCT
GGTGAAAACCACACTCCTCAAAATTCTGGCCAGCTCCTTCCACCTCAATA
GATTCTTCGATCGTATCCAAGAATCCGTGTTTCACCATAGCGTGCTCCAG
ACACTCGCCGGCAGACCCGTTGTGGAGCTGGCTCAAGGTATTAGCAGAAC
CGAGAGCCAAGATGGAGCCGGACAAGTTTCCTTTATGGAGCACACAAAGA
CCCAAAACAAGAAAGTGGTCGACGTGGGCAAGCTGCACCAGATGAAGCAA
GAAAAGGTGCCCGCTTGGACAATGCAGCTGCTCGTGGATGTCGTGTCCAA
CTCCGGTTTATCCACAATGTCCGGCATGCTGGACGAAGACATGGTGGAGG
GCGGAGTGGAGCTGGATGACGATGAGATCACCAACGAGGAACAAGCTATT
GCCACCGCCGTTCGTATCTTCGACAATATCGTGCAAGATAAGGTGGACCA
GAGCTACATTGACAGAGTCGACCTCCATAGGTTTTTAATCTGGGAGGAGG
TCGATCATTTATTCCCTTTATTCGAGGTCAATGAGAAGGGCCAAATCTCT
TTAAAGGCCTTTGCCAAGTGGGTCGTCAAGGTGTACAATGATCAAGCCGC
TTTAAAGCACGCCCTCAACGACAACAAGACCGCCGTGAAGCAACTGAATA
AGCTCGTGACCGCTATTCTGATTGTGATGATGATCGTGATTTGGCTGATC
GTCACCGGCATCGCCACAACAAAACTGATCGTGCTGCTGAGCAGCCAGCT
CGTCGTCGCTGCCTTTATCTTCGGCAACACTTGTAAAACAATCTTCGAGG
CCATCATCTTCGTCTTCGTGATGCACCCCTTTGACGTGGGAGATCGTTGT
GTCATCGACGGAAACAAAATGCTCGTGGAGGAGATGAACATCCTCACCAC
CGTCTTTTTAAAATGGGATAAAGAGAAGGTCTATTACCCCAACTCCATTT
TATGCACCAAGGCTATCGGCAATTTCTTTCGTAGCCCCGATCAAGGCGAC
GTTTTAGAGTTCTCCGTCGACTTCACAACACCCGTTTTAAAAATTGGCGA
TTTAAAGGACAGAATCAAGATGTACCTCGAACAGAATTTAAATTTCTGGC
ACCCCCAACACAACATGGTGGTCAAGGAGATCGAGAACGTCAACAAGATT
AAGATGGCTCTGTTCGTGAACCACACCATCAACTTCCAAGATTTCGCCGA
AAAAAATAGGAGGAGATCCGAACTGGTTTTAGAACTGAAAAAGATTTTCG
AAGAGCTGGACATCAAGTACAACTTATTACCTCAAGAAATTTCCATTCGT
AATATGTGA.

By “FLYC2” or “DmFLYC2” is meant a mechanosensory polypeptide capable of conferring ultrasound sensitivity on a cell, e.g., a neuron, and having at least about 85% amino acid sequence identity to the DmFLYC2 polypeptide sequence provided below, a fragment thereof, or a human ortholog thereof, and having the biological activity described herein. In embodiments, the mechanosensory polypeptide has at least about 90%, at least about 95%, or at least about 98% amino acid sequence identity to the DmFLYC2 polypeptide sequence provided below, a fragment thereof, or a human ortholog thereof, and has the biological activity described herein. In some embodiments, the DmFLYC2 polypeptide is substantially identical to the DmFLYC2 polypeptide sequence provided below or a functional variant, isoform, homolog, or ortholog having substantial identity thereto. In some embodiments, the DmFLYC2 polypeptide is a functional homolog, isoform, ortholog, or fragment of the DmFLYC2 polypeptide sequence provided below. In some embodiments, the DmFLYC2 polypeptide is or includes the DmFLYC2 polypeptide sequence provided immediately below.

DmFLYC2 polypeptide sequence:
(SEQ ID NO: 7)
MEGVRNPLRNSFNKAHEAEPQRKKNLEQEERLILLQHRNDPNSQSFSSED
PNSLLLQVKVEVAGSCDPAKTAVPTKPPVSPGGGGNLIWRDSSYDFRNDV
VKGCSRDTDDDSGEFDFQKHRVAEEEDEGEERDPESQTLSPVSESPHEYG
KITPRGAAKVSFKESELVHRRPSDGGVFAADGVSASVRDEEVVMCTSNAS
CQRKSTSTRVKTKSRLLDPPDDGDTRSGRILRSGLMPRSEDHEDEDPFSG
EDIPEEYKKMKFSFLSAVELVSLLLIIAGLVCSVVIPVVRRVTVWDMQLW
KWEVMVLVLICGGLVSGWLIRFVVFFIERNFLLRKRVLYFVYGLRRAVQR
CLWLGWVLIAWRLILDKKVEKETNSRSLLYVTKILVCLVVGTLIWLLKTL
LVKVLAMSFHVSTFFDRIQEALFDQYVIETLSGPPTIEIQHVKEDEDQVM
LEVQKLQSAGLSIPAELKATCLPNVNVNGKPVGSDPGPTPGVGKSPRSGV
IGKSPRFSRAMPEKEEGAGGITIDHLHRLNQKNISAWNMKRLMNIVRYGV
LSTLDEQILESGIEDEPSLHIKNENQAKAAAKRLFKNVARPGSKCIYLED
LMRFMREDEAARTMRAIEGSAESKGISKIALKNWVVNVFRERRALALSLN
DTKTAVNKLHQLLNFIVGFTIAIIWLLILGVPMTHFFVFITSQLLLLTFM
FGNTFKTTFEAIIFLFVMHPFDVGDRCEVEGVQMIVEEMNILTTVFLRYD
NLKITYPNSVLATKPINNYYRSPEMGDSVDFCVHISTPVEKIVVMKERIT
RYMESRRDHWRPSPKVVMREVEDMNRLKFSVWMCHTMNHQDMGERWARRE
LLVVEMVKIFKELDVQYRMLPHDVNVRTMPSLVCDRLPSNWITCTGK.

By “DmFLYC2 polynucleotide” or “FLYC2 polynucleotide” is meant a nucleic acid molecule encoding a DmFLYC2 polypeptide. In particular embodiments, the codons of the DmFLYC2 polynucleotide are optimized for expression in an organism of interest or in the cells of an organism of interest (e.g., optimized for human expression or expression in human cells, mammalian expression or mammalian cell expression, plant expression or plant cell expression). The sequence of an exemplary DmFLYC2 polynucleotide is provided immediately below. In some embodiments, the DmFLYC2 nucleic acid molecule is substantially identical to the DmFLYC2 nucleic acid molecule provided below or a functional variant, ortholog, or homolog having substantial identity thereto. In some embodiments, the DmFLYC2 nucleic acid molecule is a nucleic acid molecule with the DmFLYC2 polynucleotide sequence provided below. In some embodiments, the DmFLYC2 nucleic acid molecule is a functional homolog, isoform, or fragment of the nucleic acid molecule with the sequence provided below. In some embodiments, the DmFLYC2 nucleic acid molecule is or includes the DmFLYC2 polynucleotide sequence provided immediately below. In some embodiments, for example, for expression in a mammalian cell, e.g., a human cell, the codon-optimized DmFLYC2 polynucleotide sequence provided below is used, or a sequence with at least 85% sequence identity thereto is used. In embodiments, a sequence with at least 90%, at least 95%, or at least 98% sequence identity thereto is used.

DmFLYC2 polynucleotide sequence (DmFLYC2
codon-optimized polynucleotide sequence):
(SEQ ID NO: 8)
ATGGAGGGCGTTCGTAACCCTTTAAGGAACTCCTTCAACAAGGCCCACGA
AGCCGAGCCTCAGAGGAAGAAGAATCTGGAACAAGAAGAGAGGCTGATTT
TACTGCAACATAGGAACGACCCCAACTCCCAGAGCTTCAGCTCCGAAGAT
CCTAACTCTTTACTGCTGCAAGTTAAGGTGGAGGTGGCCGGAAGCTGCGA
TCCCGCTAAAACCGCCGTGCCCACAAAACCCCCCGTTAGCCCCGGTGGCG
GCGGAAATTTAATCTGGAGAGACAGCAGCTACGACTTTCGTAATGACGTG
GTGAAGGGCTGCTCCAGAGACACCGACGACGACTCCGGCGAATTCGACTT
CCAGAAGCATCGTGTGGCCGAGGAGGAGGACGAAGGCGAAGAAAGAGATC
CCGAGTCCCAGACACTCAGCCCCGTTTCCGAGTCCCCCCACGAGTATGGC
AAAATCACCCCCAGAGGAGCCGCTAAAGTGAGCTTCAAGGAGAGCGAGCT
GGTGCACAGAAGGCCTAGCGATGGCGGAGTGTTTGCCGCCGATGGAGTGA
GCGCCAGCGTGAGGGACGAGGAGGTCGTGATGTGCACCTCCAACGCCTCT
TGTCAGAGGAAGAGCACAAGCACTCGTGTGAAAACCAAGTCTCGTCTGCT
CGATCCTCCCGATGACGGCGACACCAGATCCGGTCGTATTTTACGTTCCG
GTTTAATGCCCAGAAGCGAAGATCACGAGGACGAAGACCCCTTTTCCGGC
GAAGACATCCCCGAAGAGTATAAGAAGATGAAGTTTAGCTTTTTATCCGC
TGTGGAGCTGGTCTCTTTATTATTAATCATCGCCGGCCTCGTCTGCAGCG
TCGTGATTCCCGTGGTGAGGAGGGTCACCGTGTGGGATATGCAGCTCTGG
AAGTGGGAAGTGATGGTGCTCGTTTTAATCTGCGGCGGTTTAGTCAGCGG
ATGGCTGATCAGATTTGTGGTCTTTTTCATCGAAAGAAACTTTTTACTGA
GGAAGAGGGTGCTCTATTTCGTGTACGGTTTAAGAAGGGCTGTGCAAAGG
TGCCTCTGGCTGGGCTGGGTGCTGATCGCTTGGAGACTGATCCTCGACAA
GAAGGTCGAGAAGGAGACCAATTCTCGTAGCTTATTATATGTCACCAAGA
TTTTAGTCTGTTTAGTGGTGGGCACTTTAATCTGGCTGCTGAAAACTTTA
CTGGTGAAGGTTTTAGCCATGTCCTTTCATGTGAGCACCTTCTTCGATCG
TATTCAAGAAGCTTTATTCGACCAGTACGTCATTGAAACACTCTCCGGCC
CCCCCACAATTGAGATCCAGCATGTCAAGGAAGACGAGGACCAAGTTATG
CTGGAGGTGCAGAAACTGCAAAGCGCCGGTTTAAGCATTCCCGCTGAGCT
CAAGGCCACTTGTTTACCCAATGTGAATGTCAATGGCAAGCCCGTTGGCA
GCGATCCCGGTCCTACACCCGGCGTGGGAAAGTCCCCTAGATCCGGAGTG
ATTGGAAAGAGCCCTCGTTTCTCTCGTGCCATGCCCGAGAAGGAGGAAGG
AGCCGGCGGCATCACCATCGATCACCTCCACAGACTCAACCAGAAGAACA
TCTCCGCTTGGAATATGAAGAGACTGATGAACATCGTGAGGTATGGCGTT
TTATCCACACTGGACGAACAGATCCTCGAGAGCGGCATCGAAGACGAACC
CTCTTTACATATCAAGAACGAGAACCAAGCCAAGGCTGCCGCCAAGAGGC
TCTTCAAAAATGTCGCTCGTCCCGGTAGCAAATGCATCTATCTGGAGGAC
CTCATGAGATTCATGAGGGAAGATGAGGCCGCTAGAACAATGAGGGCTAT
CGAGGGTTCTGCCGAGTCCAAAGGCATCTCCAAGATCGCCCTCAAGAATT
GGGTCGTGAATGTGTTCAGAGAGAGGAGGGCTTTAGCTTTATCTTTAAAT
GACACCAAGACCGCCGTGAACAAGCTGCACCAGTTATTAAACTTCATCGT
GGGCTTCACAATCGCCATCATCTGGCTCCTCATTTTAGGCGTGCCCATGA
CCCACTTCTTTGTGTTCATTACCAGCCAGTTATTACTCCTCACCTTCATG
TTCGGAAATACATTCAAAACAACATTTGAGGCCATTATTTTTCTGTTTGT
CATGCATCCTTTCGACGTGGGCGACAGATGTGAGGTGGAAGGAGTGCAGA
TGATCGTGGAAGAGATGAACATTTTAACCACCGTCTTTTTAAGGTACGAC
AATTTAAAGATCACCTATCCCAATAGCGTTTTAGCTACCAAGCCCATCAA
CAATTATTACAGAAGCCCCGAAATGGGAGACAGCGTCGACTTCTGCGTCC
ACATCTCCACCCCCGTCGAAAAGATTGTCGTCATGAAGGAGAGGATCACA
AGGTACATGGAGTCTCGTAGGGACCACTGGAGACCTTCCCCCAAAGTGGT
CATGAGGGAGGTGGAGGATATGAATAGACTCAAGTTCTCCGTCTGGATGT
GCCACACAATGAACCACCAAGACATGGGCGAGAGATGGGCTCGTAGGGAG
CTGCTGGTCGTGGAGATGGTCAAGATCTTCAAGGAGCTCGACGTGCAGTA
TCGTATGCTGCCCCACGATGTGAACGTGAGAACCATGCCCAGCCTCGTGT
GCGACAGACTGCCTAGCAATTGGATCACATGCACCGGAAAATGA.

By “DmOSCA” is meant a mechanosensory polypeptide capable of conferring ultrasound sensitivity on a cell, e.g., a neuron, and having at least about 85% amino acid sequence identity to the DmOSCA polypeptide sequence provided below, a fragment thereof, or a human ortholog thereof, and having the biological activity described herein. In embodiments, the mechanosensory polypeptide has at least about 90%, at least about 95%, or at least about 98% amino acid sequence identity to the DmOSCA polypeptide sequence provided below, a fragment thereof, or a human ortholog thereof, and has the biological activity described herein. In some embodiments, the DmOSCA polypeptide is substantially identical to the DmOSCA polypeptide sequence provided below or a functional variant, isoform, homolog, or ortholog having substantial identity thereto. In some embodiments, the DmOSCA polypeptide is a functional homolog, isoform, ortholog, or fragment of the DmOSCA polypeptide sequence provided below. In some embodiments, the DmOSCA polypeptide is or includes the DmOSCA polypeptide sequence provided immediately below.

DmOSCA polypeptide sequence:
(SEQ ID NO: 9)
MESNPEYIASLGDIVVAAVINIFFAFVFFIAFAIFRIQPVNDRVYYTKWY
LRGLRSSSTNPDAFVRKCVNLSFGSYLKFLNWMPAALQMPETELIQHAGL
DSAVYLRIYLVGLKIFIPITILALSIVIPVNWTDGGLEKSKLIAFNNLDK
LSISNIRPGSEKFWTHIGMAYTVTFWACYILKKEYESIESMRLQFLASSG
RKPEQFTVLVRNVPLDSDESTSELVEHFFKVNHPDDYLTRQVIYDANVLT
DLVRERKKKQMWLNFYQLKYTRSQSRKPFCKTGFLGLWGTKVDAIDYYTM
EVERLSKEISSKREMIANDTKAVMLAAFVSFKTRRGAAICAHTQQARNPT
LWLTQWAPEPRDIYWRNLAIPYASLSIRKLIVSVTFFFLATFFMIPIAFV
QSLANIEGIEKALPFLRPVIEARFVKSIIQGFLPGIVLKIFLTFLPSILM
MMCKSEGIISLSALERRAAARYYVFLLINVFLGSIVTGTAFEQLNNILHE
TANAIPETIGAAIPMKVTFFITYTMVDGWAGMAAEILRLKPLICYHLKVC
FLVNTEKDKEEAMNPQSFGFNTREPQIQLYFLVALVYAVAAPILLPFIVL
LFSLGYIVYRHQIINVYNQEYESGAAFWPDVHKRIVVALVVSQLLLLGLL
STKKASHSTPLLVALPVLTISFHYLCKGRFLPAFVTHPLQEATLKDSMDL
AREPGLHFKRYLQNAYTHPLLKVGDNAETDEAFQEVEQGCQLVQTKRQLW
RTFS.

By “DmOSCA polynucleotide” is meant a nucleic acid molecule encoding a DmOSCA polypeptide. In particular embodiments, the codons of the DmOSCA polynucleotide are optimized for expression in an organism of interest or in the cells of an organism of interest (e.g., optimized for human expression or expression in human cells, mammalian expression or mammalian cell expression, plant expression or plant cell expression). The sequence of an exemplary DmOSCA polynucleotide is provided immediately below. In some embodiments, the DmOSCA nucleic acid molecule is substantially identical to the DmOSCA nucleic acid molecule provided below or a functional variant, ortholog, or homolog having substantial identity thereto. In some embodiments, the DmOSCA nucleic acid molecule is a nucleic acid molecule with the DmOSCA polynucleotide sequence provided below. In some embodiments, the DmOSCA nucleic acid molecule is a functional homolog, isoform, or fragment of the nucleic acid molecule with the sequence provided below. In some embodiments, the DmOSCA nucleic acid molecule is or includes the DmOSCA polynucleotide sequence provided immediately below. In some embodiments, for example, for expression in a mammalian cell, e.g., a human cell, the codon-optimized DmOSCA polynucleotide sequence provided below is used, or a sequence with at least 85% sequence identity thereto is used. In embodiments, a sequence with at least 90%, at least 95%, or at least 98% sequence identity thereto is used.

DmOSCA polynucleotide sequence (DmOSCA
codon-optimized polynucleotide sequence):
(SEQ ID NO: 10)
ATGGAGAGCAACCCCGAATATATTGCTAGCCTCGGCGATATCGTGGTCGC
TGCCGTCATCAACATCTTCTTCGCCTTTGTGTTTTTTATCGCTTTTGCCA
TCTTCAGAATCCAGCCCGTGAACGATAGAGTGTACTACACCAAGTGGTAT
CTGAGAGGACTGAGGTCCTCCAGCACAAACCCCGACGCCTTTGTGAGGAA
GTGCGTGAATCTGAGCTTTGGCAGCTATCTGAAGTTTCTGAACTGGATGC
CCGCCGCCCTCCAGATGCCCGAGACAGAGCTGATTCAGCATGCTGGACTC
GATTCCGCCGTGTACCTCAGAATCTATCTCGTCGGACTGAAGATCTTCAT
CCCCATCACCATTCTGGCCCTCAGCATCGTGATTCCCGTGAACTGGACCG
ATGGCGGCCTCGAGAAGTCCAAGCTGATTGCTTTTAACAACCTCGACAAG
CTGTCCATCTCCAACATTAGACCCGGAAGCGAGAAGTTTTGGACCCACAT
CGGCATGGCCTATACCGTCACCTTCTGGGCTTGCTATATTCTGAAAAAAG
AGTACGAGAGCATCGAAAGCATGAGGCTCCAGTTTCTCGCCAGCAGCGGA
AGGAAGCCCGAGCAGTTTACCGTGCTGGTGAGGAACGTCCCTCTGGATAG
CGATGAATCCACCAGCGAACTGGTCGAACACTTCTTCAAGGTGAACCACC
CCGATGACTATCTGACAAGGCAAGTGATTTACGACGCCAACGTGCTGACC
GACCTCGTGAGGGAGAGGAAGAAAAAGCAGATGTGGCTCAACTTCTACCA
GCTGAAGTACACAAGAAGCCAGTCTAGAAAGCCCTTTTGCAAGACCGGCT
TCCTCGGACTGTGGGGCACAAAGGTGGACGCCATCGACTACTACACAATG
GAGGTGGAGAGACTCAGCAAGGAGATCAGCTCCAAAAGGGAGATGATCGC
TAATGACACCAAGGCTGTCATGCTCGCCGCCTTCGTCAGCTTTAAGACAA
GGAGGGGCGCTGCCATTTGCGCTCATACACAGCAAGCCAGAAACCCTACC
CTCTGGCTGACACAGTGGGCCCCCGAACCTAGGGACATCTACTGGAGGAA
TCTGGCCATCCCCTACGCCTCTCTGAGCATTAGAAAACTGATCGTGAGCG
TGACCTTCTTCTTTCTGGCTACCTTCTTCATGATCCCCATCGCTTTCGTG
CAATCTCTGGCCAACATCGAAGGAATCGAGAAAGCCCTCCCCTTTCTGAG
GCCCGTCATTGAAGCTAGATTCGTGAAGTCCATCATCCAAGGCTTTCTGC
CCGGCATCGTGCTCAAGATTTTTCTGACCTTTCTCCCCAGCATTCTGATG
ATGATGTGCAAGAGCGAGGGAATCATTTCTCTGAGCGCTCTCGAGAGGAG
AGCTGCCGCTAGATACTACGTCTTTCTGCTGATTAACGTCTTTCTGGGCA
GCATTGTGACCGGCACCGCCTTCGAACAACTCAACAACATTCTGCACGAA
ACAGCCAACGCTATCCCCGAGACCATTGGCGCTGCCATCCCCATGAAAGT
CACCTTTTTTATCACCTACACCATGGTCGATGGCTGGGCCGGCATGGCTG
CCGAGATCCTCAGACTCAAACCTCTGATCTGTTACCATCTGAAGGTGTGT
TTTCTGGTGAACACCGAGAAGGACAAGGAGGAGGCTATGAATCCTCAGTC
CTTCGGCTTCAACACCAGAGAGCCCCAAATCCAGCTCTATTTTCTGGTGG
CTCTGGTCTACGCTGTGGCTGCCCCCATTCTGCTGCCCTTTATCGTCCTC
CTCTTCTCCCTCGGCTACATCGTCTACAGACATCAGATCATCAATGTGTA
CAACCAAGAGTACGAGTCCGGAGCCGCTTTCTGGCCCGATGTGCATAAGA
GGATTGTCGTGGCTCTGGTGGTCAGCCAGCTGCTGCTGCTCGGACTGCTC
AGCACCAAGAAAGCTAGCCATTCCACACCTCTGCTGGTGGCTCTGCCCGT
GCTGACAATCTCCTTCCACTATCTGTGTAAGGGCAGATTTCTGCCCGCCT
TCGTGACACATCCTCTGCAAGAGGCCACACTGAAAGACTCCATGGATCTG
GCTAGGGAGCCCGGACTGCACTTTAAGAGGTATCTGCAGAACGCTTACAC
CCACCCTCTGCTGAAGGTGGGCGATAATGCTGAAACCGACGAAGCCTTCC
AAGAGGTGGAACAAGGCTGCCAACTGGTGCAAACCAAAAGGCAGCTGTGG
AGAACCTTTAGCTGA.

By “DcFLYC1.1” is meant a mechanosensory polypeptide capable of conferring ultrasound sensitivity on a cell, e.g., a neuron, and having at least about 85% amino acid sequence identity to the DcFLYC1.1 polypeptide sequence provided below, a fragment thereof, or a human ortholog thereof, and having the biological activity described herein. In embodiments, the mechanosensory polypeptide has at least about 90%, at least about 95%, or at least about 98% amino acid sequence identity to the DcFLYC1.1 polypeptide sequence provided below, a fragment thereof, or a human ortholog thereof, and has the biological activity described herein. In some embodiments, the DcFLYC1.1 polypeptide is substantially identical to the DcFLYC1.1 polypeptide sequence provided below or a functional variant, isoform, homolog, or ortholog having substantial identity thereto. In some embodiments, the DcFLYC1.1 polypeptide is a functional homolog, isoform, ortholog, or fragment of the DcFLYC1.1 polypeptide sequence provided below. In some embodiments, the DcFLYC1.1 polypeptide is or includes the DcFLYC1.1 polypeptide sequence provided immediately below.

DcFLYC1.1 polypeptide sequence:
(SEQ ID NO: 11)
MASNTNISQQGGEINFEKQMAHRRRHEQLAIQIPVKTASQTFRFNEEVDT
RSKFSPAPDITMFYPQPSPNKPPRVPNRTLTRRSTTLKTKPKSRFGEPSL
PIDPAALWELAPNSPTPSFREATPSSNNHRFSVGRGSSFAKGVTPRVAAS
SQRGETTIEGPDEKEVYERVTAQLSARDKKRMTVKLLIELAIFLFVSGCL
ISSLTIHGLKVRKIYGLPIWRLFLFLLVILSGMLVTHWMIHVVVFLIEWK
FLLKKNVVYFTHGLKTSVEVFIWITLILATWGLLIEPDVRHTNRIRNALD
FITWTLLSLLLGSFLWLIKTIMIKTLAASFHLNRFFDRIQESIFHHYVLQ
TLSGRPVVELASGVLTRTETHNGMVSFTEHTKTHKEKKMVDMGKLHQMKQ
EKVPDWTMQLLVDVVSNSGLSTMSGILDEDMAEGGVELDDDEITSEEQAI
ATAVRIFYNIVKDKDDQSYIDRKDLHRFLICEEVDLVFPLFEVKDKDQIN
LKAFSKWVVKLFKERQALKHALNDNKTAVKQLDKLVTSILIVVIIAVWLL
LTEIMTTKVLLFFSSQLLVAVFVFGNTCKTIFEAIIFVFVMHPFDVGDRC
VVDGTMMLVEEMNILTTVFLKWDKEKVYYPNAVLSTKAIGNYYRSPDQVD
SLEFSIDFRTPLSKIGEIKERIKKYLHQNPHLWHPNHNFVVKEIENVNKI
KMQLIFNHTINFQEFPERMKRRSELVLELKKIFEELDIKYNLLPQEVILN
KVSP.

By “DcFLYC1.1 polynucleotide” is meant a nucleic acid molecule encoding a DcFLYC1.1 polypeptide. In particular embodiments, the codons of the DcFLYC1.1 polynucleotide are optimized for expression in an organism of interest or in the cells of an organism of interest (e.g., optimized for human expression or expression in human cells, mammalian expression or mammalian cell expression, plant expression or plant cell expression). The sequence of an exemplary DcFLYC1.1 polynucleotide is provided immediately below. In some embodiments, the DcFLYC1.1 nucleic acid molecule is substantially identical to the DcFLYC1.1 nucleic acid molecule provided below or a functional variant, ortholog, or homolog having substantial identity thereto. In some embodiments, the DcFLYC1.1 nucleic acid molecule is a nucleic acid molecule with the DcFLYC1.1 polynucleotide sequence provided below. In some embodiments, the DcFLYC1.1 nucleic acid molecule is a functional homolog, isoform, or fragment of the nucleic acid molecule with the sequence provided below. In some embodiments, the DcFLYC1.1 nucleic acid molecule is or includes the DcFLYC1.1 polynucleotide sequence provided immediately below. In some embodiments, for example, for expression in a mammalian cell, e.g., a human cell, the codon-optimized DcFLYC1.1 polynucleotide sequence provided below is used, or a sequence with at least 85% sequence identity thereto is used. In embodiments, a sequence with at least 90%, at least 95%, or at least 98% sequence identity thereto is used.

DcFLYC1.1 polynucleotide sequence (DcFLYC1.1
codon-optimized polynucleotide sequence):
(SEQ ID NO: 12)
ATGGCTAGCAACACAAATATTTCCCAGCAAGGCGGCGAGATCAACTTCGA
AAAGCAGATGGCCCACAGAAGGAGACATGAGCAGCTGGCCATCCAAATCC
CCGTGAAAACCGCCAGCCAGACCTTCAGATTCAACGAGGAAGTGGACACA
AGAAGCAAGTTCAGCCCCGCCCCCGACATTACCATGTTCTACCCCCAGCC
TAGCCCCAACAAACCTCCTAGGGTGCCCAATAGGACACTCACCAGAAGGA
GCACCACACTGAAGACCAAACCCAAATCTAGATTCGGCGAACCTTCTCTG
CCTATCGATCCCGCTGCCCTCTGGGAACTGGCTCCCAATTCCCCTACCCC
CAGCTTTAGAGAGGCCACCCCCTCCTCCAACAACCATAGATTCTCCGTGG
GAAGAGGCAGCAGCTTTGCTAAGGGAGTGACCCCTAGAGTCGCCGCCAGC
AGCCAAAGAGGCGAGACAACAATCGAGGGCCCCGACGAGAAAGAAGTGTA
CGAGAGGGTCACAGCCCAGCTGAGCGCTAGGGATAAGAAGAGGATGACCG
TCAAGCTGCTGATCGAGCTGGCCATCTTTCTGTTCGTCAGCGGCTGCCTC
ATCTCCAGCCTCACAATTCACGGACTGAAGGTGAGAAAGATCTACGGACT
GCCTATTTGGAGGCTGTTCCTCTTTCTCCTCGTCATTCTGAGCGGAATGC
TGGTGACACACTGGATGATCCATGTCGTGGTGTTTCTGATCGAATGGAAG
TTTCTGCTGAAGAAGAATGTGGTCTACTTCACCCACGGACTGAAGACCTC
CGTGGAGGTCTTCATTTGGATCACACTGATCCTCGCCACATGGGGACTGC
TCATCGAGCCCGACGTCAGACATACCAATAGAATTAGAAATGCCCTCGAC
TTCATCACATGGACACTGCTGTCTCTGCTGCTCGGCAGCTTTCTCTGGCT
GATCAAGACCATCATGATTAAGACACTCGCCGCCTCCTTCCATCTGAATA
GATTTTTCGATAGAATCCAAGAGTCCATCTTCCACCACTACGTGCTGCAG
ACACTCTCCGGCAGACCCGTCGTCGAACTGGCTTCCGGAGTGCTGACAAG
GACCGAGACACACAATGGCATGGTCAGCTTTACCGAGCACACAAAGACCC
ACAAGGAAAAGAAGATGGTGGACATGGGAAAGCTGCACCAGATGAAGCAA
GAGAAGGTCCCCGACTGGACAATGCAGCTGCTGGTGGATGTCGTGAGCAA
CTCCGGCCTCAGCACCATGTCCGGAATTCTGGACGAGGACATGGCTGAGG
GAGGCGTGGAGCTCGATGACGACGAGATCACCTCCGAGGAGCAAGCCATT
GCTACCGCCGTCAGAATCTTCTATAATATTGTCAAGGACAAGGACGACCA
GTCCTACATCGACAGAAAGGACCTCCATAGATTTCTGATCTGTGAGGAGG
TGGATCTGGTGTTCCCTCTGTTTGAGGTGAAAGACAAAGACCAGATTAAT
CTGAAGGCCTTCAGCAAGTGGGTCGTGAAGCTCTTTAAAGAGAGGCAAGC
CCTCAAGCACGCCCTCAACGACAACAAGACCGCCGTGAAACAGCTCGATA
AGCTGGTGACCTCCATTCTGATTGTGGTGATTATCGCCGTGTGGCTGCTG
CTGACCGAGATTATGACCACCAAAGTGCTGCTGTTCTTCTCCTCCCAGCT
CCTCGTGGCCGTGTTTGTCTTCGGCAACACATGCAAGACAATCTTCGAGG
CCATTATCTTTGTGTTCGTCATGCATCCCTTCGACGTGGGCGATAGATGT
GTGGTGGACGGCACCATGATGCTGGTCGAAGAGATGAACATCCTCACCAC
CGTCTTTCTGAAGTGGGACAAGGAAAAGGTGTACTACCCCAACGCTGTCC
TCTCCACCAAGGCTATTGGCAATTACTATAGAAGCCCCGACCAAGTGGAT
TCTCTGGAGTTCTCCATCGACTTTAGAACACCCCTCTCCAAAATTGGAGA
GATCAAAGAGAGGATTAAGAAATATCTCCATCAGAACCCCCATCTGTGGC
ACCCCAACCACAACTTCGTGGTGAAGGAGATCGAAAATGTCAATAAGATT
AAGATGCAGCTGATCTTTAATCACACAATTAATTTCCAAGAGTTTCCCGA
GAGGATGAAGAGGAGAAGCGAGCTGGTGCTGGAGCTGAAGAAGATCTTCG
AGGAGCTGGACATCAAGTATAATCTGCTGCCCCAAGAGGTCATTCTCAAC
AAGGTGAGCCCTTGA.

By “DcFLYC1.2” is meant a mechanosensory polypeptide capable of conferring ultrasound sensitivity on a cell, e.g., a neuron, and having at least about 85% amino acid sequence identity to the DcFLYC1.2 polypeptide sequence provided below, a fragment thereof, or a human ortholog thereof, and having the biological activity described herein. In embodiments, the mechanosensory polypeptide has at least about 90%, at least about 95%, or at least about 98% amino acid sequence identity to the DcFLYC1.2 polypeptide sequence provided below, a fragment thereof, or a human ortholog thereof, and has the biological activity described herein. In some embodiments, the DcFLYC1.2 polypeptide is substantially identical to the DcFLYC1.2 polypeptide sequence provided below or a functional variant, isoform, homolog, or ortholog having substantial identity thereto. In some embodiments, the DcFLYC1.2 polypeptide is a functional homolog, isoform, ortholog, or fragment of the DcFLYC1.2 polypeptide sequence provided below. In some embodiments, the DcFLYC1.2 polypeptide is or includes the DcFLYC1.2 polypeptide sequence provided immediately below.

DcFLYC1.2 polypeptide sequence:
(SEQ ID NO: 13)
MASNTNISQQGGEINFEKQMAHRRRHEQLAIQIPVKTASQTFPFNEEVDT
TRSKFSPAPSFREATPSSNNHRASVGRGSSFVKGVTPRVAASSRRGETTI
EGPDEREVYERVTAQLSARDKKRMTVKLLIELAVFLFVSGCLISSLTIHG
LKVRIICGLPIWRLFLFLLVILSGMLVTHWMLHVVVFLIEWKFLLKKNVV
YFTHGLKTSVEVFIWITLILATWALLIEPDVRHTNRIRNALDFITWTLLS
LLLCSFLWLIKTIMIKTLAASFHLNRFFDRIQESIFHHYVLQTLSGRPVV
ELASGVLTRTETHNGMVSFTEHTKTHTEKKMVDMGKLHQMKQEKVPDWTM
QLLVDVVSNSGLSTMSGILDEDMAEGGVELDDDEITSEEQAIATAVRIFY
NIVKDKDDQTYIDRKDLHRFLICEEVDLVFPLFEVKDKDQISLKAFSKWV
VKLFKERQALKHALNDNKTAVKQLDKLVTSILIVVIIAVWLLLTELMTTK
VLLFFTSQLLVAVFVFGNTCKTIFEAIIFVFVMHPFDVGDRCVIDGTTML
VEEMNILTTVFLKWDKEKVYYPNAVLSTKAIGNYYRSPDQVDSLEFSIDF
RTPLSKIGEIKERIKKYLHQNPHLWHPNHNLVVKEIENVNKIKTQLIFNH
TMNFQEFPERMKRRTELVLELKKIFEELDIKYNLLPQEVILNNVGP.

By “DcFLYC1.2 polynucleotide” is meant a nucleic acid molecule encoding a DcFLYC1.2 polypeptide. In particular embodiments, the codons of the DcFLYC1.2 polynucleotide are optimized for expression in an organism of interest or in the cells of an organism of interest (e.g., optimized for human expression or expression in human cells, mammalian expression or mammalian cell expression, plant expression or plant cell expression). The sequence of an exemplary DcFLYC1.2 polynucleotide is provided immediately below. In some embodiments, the DcFLYC1.2 nucleic acid molecule is substantially identical to the DcFLYC1.2 nucleic acid molecule provided below or a functional variant, ortholog, or homolog having substantial identity thereto. In some embodiments, the DcFLYC1.2 nucleic acid molecule is a nucleic acid molecule with the DcFLYC1.2 polynucleotide sequence provided below. In some embodiments, the DcFLYC1.2 nucleic acid molecule is a functional homolog, isoform, or fragment of the nucleic acid molecule with the sequence provided below. In some embodiments, the DcFLYC1.2 nucleic acid molecule is or includes the DcFLYC1.2 polynucleotide sequence provided immediately below. In some embodiments, for example, for expression in a mammalian cell, e.g., a human cell, the codon-optimized DcFLYC1.2 polynucleotide sequence provided below is used, or a sequence with at least 85% sequence identity thereto is used. In embodiments, a sequence with at least 90%, at least 95%, or at least 98% sequence identity thereto is used.

DcFLYC1.2 polynucleotide sequence (DmFLYC1.2
codon-optimized polynucleotide sequence):
(SEQ ID NO: 14)
ATGGCTAGCAACACAAATATTTCCCAGCAAGGCGGCGAGATCAACTTCGA
AAAGCAGATGGCTCATAGAAGAAGGCATGAGCAACTCGCTATCCAGATCC
CCGTGAAGACAGCCAGCCAGACCTTCCCCTTCAACGAGGAAGTCGATACC
ACAAGAAGCAAGTTCAGCCCCGCCCCCGACATCACCATGTTCTACCCCCA
ACCTAGCCCTAACAAACCCCCTAGGGTGCCCAATAGGAATCTGTCTAGAA
GATCCACCACACTGAAGACAAAGCCCAAGTCTAGATTCGGCGAACCCAGC
CTCCCTATTGACCCCGCCGCTCTGTGGGAACTGGCCCCCAACAGCCCCGC
TCCCTCCTTTAGGGAAGCCACACCCTCCAGCAACAACCACAGAGCCTCCG
TGGGAAGGGGCAGCTCCTTCGTGAAGGGAGTCACACCTAGGGTGGCCGCC
AGCTCTAGAAGAGGCGAAACCACAATTGAAGGCCCCGACGAGAGAGAGGT
CTATGAGAGAGTGACAGCTCAGCTGAGCGCCAGAGATAAGAAGAGAATGA
CCGTGAAGCTCCTCATTGAGCTCGCCGTCTTTCTGTTTGTGTCCGGATGT
CTGATCTCCAGCCTCACAATTCACGGACTGAAGGTGAGAATTATCTGTGG
ACTGCCCATCTGGAGACTGTTTCTGTTTCTGCTGGTGATCCTCTCCGGAA
TGCTGGTCACACACTGGATGCTCCATGTCGTCGTGTTCCTCATCGAATGG
AAGTTTCTGCTGAAAAAGAATGTCGTGTACTTCACCCACGGACTGAAAAC
CTCCGTCGAGGTGTTCATCTGGATCACACTGATTCTGGCCACATGGGCTC
TGCTGATTGAGCCCGACGTGAGACACACCAATAGAATCAGAAACGCCCTC
GACTTCATCACATGGACACTGCTGTCTCTGCTGCTCTGCTCCTTTCTCTG
GCTGATCAAGACCATCATGATCAAGACACTGGCCGCTTCCTTCCATCTGA
ATAGGTTCTTTGATAGAATCCAAGAGTCCATCTTCCATCACTACGTGCTC
CAGACCCTCTCCGGAAGGCCCGTGGTCGAGCTCGCTTCCGGCGTCCTCAC
CAGAACAGAGACCCACAACGGAATGGTCAGCTTCACCGAGCATACCAAGA
CCCACACAGAGAAGAAGATGGTGGACATGGGCAAGCTCCACCAAATGAAG
CAAGAGAAGGTCCCCGACTGGACCATGCAGCTGCTGGTGGATGTGGTGAG
CAATAGCGGACTGAGCACCATGTCCGGCATTCTGGACGAAGACATGGCCG
AAGGCGGAGTCGAACTGGACGATGACGAGATCACCTCCGAAGAGCAAGCC
ATTGCCACCGCTGTGAGGATTTTCTACAACATCGTCAAGGACAAAGACGA
CCAGACCTACATCGATAGAAAGGATCTGCATAGATTTCTGATCTGCGAAG
AAGTGGACCTCGTGTTCCCCCTCTTTGAAGTGAAGGACAAGGACCAGATC
TCTCTGAAGGCCTTTAGCAAGTGGGTGGTGAAGCTCTTCAAGGAGAGGCA
AGCCCTCAAGCACGCCCTCAACGACAATAAGACCGCCGTCAAGCAGCTGG
ATAAACTCGTGACAAGCATCCTCATCGTGGTCATCATCGCCGTCTGGCTG
CTGCTGACCGAACTCATGACCACCAAGGTGCTGCTGTTCTTCACCAGCCA
ACTGCTCGTGGCCGTGTTTGTGTTTGGAAATACATGTAAAACAATCTTTG
AGGCTATCATCTTCGTGTTCGTGATGCACCCTTTCGACGTGGGCGATAGA
TGTGTGATCGATGGAACCACCATGCTGGTCGAGGAGATGAATATCCTCAC
CACAGTGTTTCTGAAGTGGGACAAAGAAAAAGTGTACTACCCCAACGCCG
TGCTGAGCACCAAAGCTATTGGAAATTACTATAGGTCCCCCGACCAAGTG
GACTCTCTGGAGTTTAGCATCGATTTTAGAACCCCTCTGTCCAAAATCGG
AGAGATTAAGGAAAGGATCAAAAAATATCTGCACCAGAATCCCCATCTGT
GGCACCCTAATCACAATCTGGTGGTCAAGGAGATCGAGAATGTGAACAAG
ATCAAAACACAACTCATTTTCAATCACACCATGAACTTCCAAGAGTTCCC
CGAGAGGATGAAGAGAAGGACAGAACTCGTGCTGGAGCTGAAGAAAATCT
TCGAGGAGCTGGACATCAAATACAATCTGCTGCCCCAAGAAGTCATTCTG
AACAACGTGGGACCCTGA.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refers to compounds that have the same basic chemical structure as a naturally occurring amino acid, i.e., an a carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions in a manner similar to a naturally occurring amino acid. The terms “non-naturally occurring amino acid” and “unnatural amino acid” refer to amino acid analogs, synthetic amino acids, and amino acid mimetics, which are not found in nature.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

“Conservatively modified variants” applies to both amino acid and nucleic acid sequences. With respect to particular nucleic acid sequences, conservatively modified variants refers to those nucleic acids which encode identical or essentially identical amino acid sequences, or where the nucleic acid does not encode an amino acid sequence, to essentially identical sequences. Because of the degeneracy of the genetic code, a large number of functionally identical nucleic acids encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein, which encodes a polypeptide also describes every possible silent variation of the nucleic acid. One of skill will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid, which encodes a polypeptide is implicit in each described sequence with respect to the expression product, but not with respect to actual probe sequences.

By “altered” is meant an increase (or enhancement), or a decrease (or reduction). An increase is any positive change, e.g., by at least about 5%, 10%, or 20%; or by about 25%, 50%, 75%, or even by 100%, 200%, 300% or more. A decrease is a negative change, e.g., a decrease by about 5%, 10%, or 20%; or by about 25%, 50%, 75%; or even an increase by 100%, 200%, 300% or more.

The terms “comprises”, “comprising”, and are intended to have the broad meaning ascribed to them in U.S. Patent Law and can mean “includes”, “including” and the like.

“Contacting” is used in accordance with its plain ordinary meaning and refers to the process of allowing at least two distinct species (e.g. chemical compounds including biomolecules, reagents, or cells) to become sufficiently proximal to react, interact, effect, affect or physically touch. It should be appreciated, however, that the resulting reaction product can be produced directly from a reaction between the added reagents or from an intermediate of one or more of the added reagents, which can be produced in the reaction mixture or under the contacting conditions. Contacting may include allowing two species to react, interact, or physically touch, wherein the two species may be a recombinant viral particle as described herein and a cell. In some embodiments, the two species are an ultrasound contrast agent that is exposed to ultrasound and a cell. In some embodiments, the two species are ultrasound and a cell.

The word “expression” or “expressed” as used herein in reference to a gene means the transcriptional and/or translational product of that gene. The level of expression of a DNA molecule in a cell may be determined on the basis of either the amount of corresponding mRNA that is present within the cell or the amount of protein encoded by that DNA produced by the cell. The level of expression of non-coding nucleic acid molecules (e.g., siRNA) may be detected by standard PCR or Northern blot methods well known in the art. See, Sambrook et al., 1989 Molecular Cloning: A Laboratory Manual, 18.1-18.88.

Expression of a transfected gene can occur transiently or stably in a cell. During “transient expression” the transfected gene is not transferred to the daughter cell during cell division. Since its expression is restricted to the transfected cell, expression of the gene is lost over time. In contrast, stable expression of a transfected gene can occur when the gene is co-transfected with another gene that confers a selection advantage to the transfected cell. Such a selection advantage may be a resistance towards a certain toxin that is presented to the cell. Expression of a transfected gene can further be accomplished by transposon-mediated insertion into to the host genome. During transposon-mediated insertion, the gene is positioned in a predictable manner between two transposon linker sequences that allow insertion into the host genome as well as subsequent excision. Stable expression of a transfected gene can further be accomplished by infecting a cell with a lentiviral vector, which after infection forms part of (integrates into) the cellular genome thereby resulting in stable expression of the gene.

The term “exogenous” (synonymous with “heterologous”) refers to a molecule, reagent, or substance (e.g., a compound, nucleic acid (polynucleotide) or protein (polypeptide or peptide) that originates or derives from a source outside of a given cell or organism. For example, an “exogenous promoter” as referred to herein is a promoter that does not originate from the source (e.g., a given cell or organism) in which it is expressed. By way of example, an “exogenous” or “heterologous” polypeptide or polynucleotide as referred to herein does not originate from the source (e.g., a given cell, tissue, organ, or organism) in which it is expressed, but is obtained or derived from a different source and is introduced or delivered into a given cell, tissue, organ, or organism by genetic or recombinant techniques and then is expressed in that given cell, tissue, organ, or organism. By way of example, an exogenous promoter may be derived from a given organism, such as a bacterium, plant, or fungus (yeast), and used in another organism or cell type, such as a mammalian cell. Conversely, the term “endogenous” (e.g., “endogenous promoter,” “endogenous protein or polypeptide,” or “endogenous polynucleotide”) refers to a molecule or substance that is native to, or originates within, a given cell, tissue, organ, or organism. The terms “heterologously expressed” or “exogenously expressed” are used interchangeably herein in reference to the expression of a heterologous or exogenous polypeptide in a cell.

By “fragment” is meant a portion of a polypeptide or nucleic acid molecule. This portion contains, for example, at least 10%, 20%, 30%, 40%, 50%, 60%, 70%, 80%, or 90% of the entire length of the reference nucleic acid molecule or polypeptide. A fragment may contain 10, 20, 30, 40, 50, 60, 70, 80, 90, or 100, 200, 300, 400, 500, 600, 700, 800, 900, or 1000 nucleotides or amino acids.

The term “gene” means the segment of DNA involved in producing a protein; it includes regions preceding and following the coding region (leader and trailer) as well as intervening sequences (introns) between individual coding segments (exons). The leader, the trailer as well as the introns include regulatory elements that are necessary during the transcription and the translation of a gene. Further, a “protein gene product” is a protein expressed from a particular gene.

“Hybridization” means hydrogen bonding, which may be Watson-Crick, Hoogsteen or reversed Hoogsteen hydrogen bonding, between complementary nucleobases. For example, adenine and thymine are complementary nucleobases that pair through the formation of hydrogen bonds.

The terms “identical” or percent “identity,” in the context of two or more nucleic acids or polypeptide sequences, refer to two or more sequences or subsequences that are the same or have a specified percentage of amino acid residues or nucleotides that are the same (i.e., about 60% identity, or 65%, 70%, 75%, 80%, 85%, 90%, 91%, 92%, 93%, 94%, 95%, 96%, 97%, 98%, 99%, or higher identity over a specified region, when compared and aligned for maximum correspondence over a comparison window or designated region) as measured using a BLAST or BLAST 2.0 sequence comparison algorithms with default parameters described below, or by manual alignment and visual inspection. Such sequences are then said to be “substantially identical” or “homologous.” This definition also refers to, or may be applied to, the compliment of a test sequence. The definition also includes sequences that have deletions and/or additions, as well as those that have substitutions. As described below, the preferred algorithms can account for gaps and the like. In an embodiment, identity exists over a region that is at least about 25 amino acids or nucleotides in length, or over a region that is 50-100 amino acids or nucleotides in length.

The terms “isolated,” “purified,” or “biologically pure” refer to material that is free to varying degrees from components which normally accompany it as found in its native state. “Isolate” denotes a degree of separation from original source or surroundings. “Purify” denotes a degree of separation that is higher than isolation. A “purified” or “biologically pure” protein is sufficiently free of other materials such that any impurities do not materially affect the biological properties of the protein or cause other adverse consequences. That is, a nucleic acid, polypeptide, or peptide is purified if it is substantially free of cellular material, viral material, or culture medium when produced by recombinant DNA techniques, or chemical precursors or other chemicals when chemically synthesized. Purity and homogeneity are typically determined using analytical chemistry techniques, for example, polyacrylamide gel electrophoresis or high performance liquid chromatography. The term “purified” can denote that a nucleic acid or protein gives rise to essentially one band in an electrophoretic gel. For a protein that can be subjected to modifications, for example, phosphorylation or glycosylation, different modifications may give rise to different isolated proteins, which can be separately purified.

By “isolated polynucleotide” is meant a nucleic acid (e.g., a DNA) that is free of the genes which, in the naturally-occurring genome of the organism from which the nucleic acid molecule as described herein is derived, flank the gene. The term therefore includes, for example, a recombinant DNA that is incorporated into a vector; into an autonomously replicating plasmid or virus; or into the genomic DNA of a prokaryote or eukaryote; or that exists as a separate molecule (for example, a cDNA or a genomic or cDNA fragment produced by PCR or restriction endonuclease digestion) independent of other sequences. In addition, the term includes an RNA molecule that is transcribed from a DNA molecule, as well as a recombinant DNA that is part of a hybrid gene encoding additional polypeptide sequence.

By an “isolated polypeptide” is meant a polypeptide as described herein that has been separated from components that naturally accompany it. Typically, the polypeptide is isolated when it is at least 50%, at least 55%, or at least 60%, by weight, free from the proteins and naturally-occurring organic molecules with which it is naturally associated. In embodiments, a preparation is at least 75%, or at least 90%, or at least 99%, by weight, a mechanosensory polypeptide as described herein. An isolated polypeptide as described herein may be obtained, for example, by extraction from a natural source, by expression of a recombinant nucleic acid encoding such a polypeptide; or by chemically synthesizing the protein. Purity can be measured by any appropriate method, for example, column chromatography, polyacrylamide gel electrophoresis, or by HPLC analysis.

By “mammal” is meant any warm-blooded animal including, but not limited to, non-human primate (monkey, ape, baboon and the like), human, cow, horse, pig, sheep, goat, mouse, rat, dog, cat, and the like. In an embodiment, the mammal is a human.

The terms “mechanosensitive”, “mechanically activated”, “mechanoreceptor”, “mechanotransduction”, “stretch-gated”, “acoustically sensitive”, and other similar terms of art as used herein are considered interchangeable and are used to refer to a cell, tissue, or polypeptide, or other material object that is sensitive to activation or inactivation by acoustical energy, such as ultrasound.

By “modulating” is meant effecting or altering the activity or function of a cell, tissue, organ, organism, or subject, for example, by subjecting the cell, tissue, organ, organism, or subject, to ultrasound stimulation. In an embodiment, the activity or function of a cell, such as an insulin-secreting cell, or a neuronal cell is modulated by applying or delivering ultrasound or ultrasound waves to the cell. In embodiments, modulating cells in a subject, e.g., in the brain or CNS of the subject, affects the subject's behavior or response to an agent, stimulus, situation, effect, or activity. In some embodiments, modulating an activity or function may cause an increase (enhancement) or decrease (inhibition or silencing) of cell activity or function, or in a subject's response or responsiveness.

“Nucleic acid” refers to deoxyribonucleotides or ribonucleotides and polymers thereof in either single- or double-stranded form, or complements thereof. The term “polynucleotide” refers to a linear sequence of nucleotides. The terms “polynucleotide” and “nucleic acid molecule” are used interchangeably herein. The term “nucleotide” typically refers to a single unit of a polynucleotide, i.e., a monomer. Nucleotides can be ribonucleotides, deoxyribonucleotides, or modified versions thereof. Examples of polynucleotides contemplated herein include single and double stranded DNA, single and double stranded RNA (including siRNA), and hybrid molecules having mixtures of single and double stranded DNA and RNA. The terms also encompass nucleic acids containing known nucleotide analogs or modified backbone residues or linkages, which are synthetic, naturally occurring, and non-naturally occurring, which have similar binding properties as the reference nucleic acid, and which are metabolized in a manner similar to the reference nucleotides. Examples of such analogs include, without limitation, phosphorothioates, phosphoramidates, methyl phosphonates, chiral-methyl phosphonates, and 2-O-methyl ribonucleotides.

Nucleic acid is “operably linked” when it is placed into a functional relationship with another nucleic acid sequence. For example, DNA for a presequence or secretory leader is operably linked to DNA for a polypeptide if it is expressed as a preprotein that participates in the secretion of the polypeptide; a promoter or enhancer is operably linked to a coding sequence if it affects the transcription of the sequence; or a ribosome binding site is operably linked to a coding sequence if it is positioned so as to facilitate translation. Generally, “operably linked” means that the DNA sequences being linked are near each other, and, in the case of a secretory leader, contiguous and in reading phase. However, enhancers do not have to be contiguous. Linking is accomplished by ligation at convenient restriction sites. If such sites do not exist, the synthetic oligonucleotide adaptors or linkers are used in accordance with conventional practice.

The term, “obtaining” as in “obtaining an agent” includes synthesizing, deriving, isolating, purchasing, or otherwise acquiring the agent, e.g., a protein, polynucleotide, or sample.

By “positioned for expression” is meant that a polynucleotide (e.g., a DNA molecule) is positioned adjacent to a DNA sequence, which directs transcription, and, for proteins, translation of the sequence (i.e., facilitates the production of, for example, a recombinant polypeptide as described herein, or an RNA molecule).

The term “plasmid” or “vector” refers to a nucleic acid molecule that encodes for genes and/or regulatory elements necessary for the expression of genes. Expression of a gene from a plasmid or vector can occur in cis or in trans. If a gene is expressed in cis, the gene and the regulatory elements are encoded by the same plasmid and vector. Expression in trans refers to the instance where the gene and the regulatory elements are encoded by separate plasmids or vectors.

As used herein, the terms “prevent,” “preventing,” “prevention,” “prophylactic treatment” and the like refer to reducing the probability of developing a disorder or condition in a subject, who does not have, but is at risk of or susceptible to developing a disorder or condition.

By “reduces” is meant a negative alteration of at least 5%, 10%, 25%, 50%, 75%, or 100%.

By “reference” or “control” is meant a standard condition. For example, an untreated cell, tissue, or organ that is used as a reference.

The terms “protein”, “peptide”, and “polypeptide” are used interchangeably to denote an amino acid polymer or a set of two or more interacting or bound amino acid polymers. The terms apply to amino acid polymers in which one or more amino acid residue is an artificial chemical mimetic of a corresponding naturally occurring amino acid, as well as to naturally occurring amino acid polymers and non-naturally occurring amino acid polymer. For specific polypeptides described herein (e.g., MscS, MscL, MscK, MscL G22S, MscS-like, MscMJ, MscMJLR, Msc-Like 3, MscSfam), the named polypeptide includes any of the polypeptide's naturally occurring forms, or variants, isoforms, or homologs that maintain the polypeptide's mechanosensory activity (e.g., within at least 50%, 80%, 90%, 95%, 96%, 97%, 98%, 99% or 100% activity compared to the native polypeptide). In some embodiments, variants or homologs have at least 90%, 95%, 96%, 97%, 98%, 99% or 100% amino acid sequence identity across the whole sequence or a portion of the polypeptide sequence (e.g. a 50, 100, 150 or 200 continuous amino acid portion) compared to a naturally occurring form. In other embodiments, the polypeptide is the polypeptide as identified by its Genbank Accession number.

Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50.

The term “recombinant” when used with reference, e.g., to a cell, or nucleic acid, protein, or vector, indicates that the cell, nucleic acid, protein or vector, has been modified by the introduction of a heterologous nucleic acid or protein or the alteration of a native nucleic acid or protein, or that the cell is derived from a cell so modified. Thus, for example, recombinant cells express genes that are not found within the native (non-recombinant) form of the cell or express native genes that are otherwise abnormally expressed, under expressed or not expressed at all. Transgenic cells and plants are those that express a heterologous gene or coding sequence, typically as a result of recombinant methods.

The terms “sonogenetics,” “sonogenetic,” “sonogenics” or “sonogenic” refer to a non-invasive approach, method, or technique to manipulate, control, or modulate the activity or function of a cell or cell type, such as a neuron (e.g., a motor neuron), that expresses a heterologous or exogenous mechanosensitive (also called mechanotransductive) channel, which is responsive to ultrasound, e.g., low-intensity ultrasound. Cell activity, such as neuronal cell activity, can be controlled or modulated by expressing a heterologous or exogenous mechanosensitive channel in a target cell, e.g., a neuron or type of neuronal cell such as a motor neuron, and subjecting the cell to ultrasound (low-intensity ultrasound), which is thereby responsive to ultrasound or ultrasound pulses. In an embodiment, the cell types are located within the mammalian brain. Target cells that express such heterologous or exogenous mechanosensitive channel is specific cells renders those cells sensitive to mechanical deformations generated by noninvasive ultrasound waves. In an embodiment, the cells are neurons in regions of the brain or in the spinal cord (central nervous system, CNS). In an embodiment the cells are in peripheral nervous system (PNS). In an embodiment, the region of the brain is the hypothalamus. In an embodiment, ultrasound is delivered or applied to the hypothalamus using an external transducer. In an embodiment, the transducer is non-invasively positioned on the head of an awake mammalian subject. In an embodiment, the transducer is a PZT-based transducer. In an embodiment, the cells are neurons in the spinal cord.

The term “subject” as used herein refers to a vertebrate organism, for example, a mammal, e.g., dog, cat, rodent, horse, bovine, rabbit, goat, non-human primate, or human. In an embodiment, a subject may be a human individual or patient.

By “substantially identical” is meant a polypeptide or nucleic acid molecule exhibiting at least 50% identity to a reference amino acid sequence (for example, any one of the amino acid sequences described herein) or nucleic acid sequence (for example, any one of the nucleic acid sequences described herein). In embodiments, such a sequence is at least 60%, or at least 80% or 85%, or at least 90%, 95% or even 99% identical at the amino acid level or nucleic acid to the sequence used for comparison.

Sequence identity is typically measured using sequence analysis software (for example, Sequence Analysis Software Package of the Genetics Computer Group, University of Wisconsin Biotechnology Center, 1710 University Avenue, Madison, Wis. 53705, BLAST, BESTFIT, GAP, or PILEUP/PRETTYBOX programs). Such software matches identical or similar sequences by assigning degrees of homology to various substitutions, deletions, and/or other modifications. Conservative substitutions typically include substitutions within the following groups: glycine, alanine; valine, isoleucine, leucine; aspartic acid, glutamic acid, asparagine, glutamine; serine, threonine; lysine, arginine; and phenylalanine, tyrosine. In an exemplary approach to determining the degree of identity, a BLAST program may be used, with a probability score between e⁻³ and e⁻¹⁰⁰ indicating a closely related sequence.

By “transformed cell” is meant a cell into which (or into an ancestor of which) has been introduced, by means of recombinant DNA techniques, a polynucleotide molecule encoding (as used herein) a polypeptide as described herein.

As used herein, the terms “treat,” treating,” “treatment,” and the like refer to reducing, abating, decreasing, ameliorating, or eliminating a disease, disorder, pathology, or condition and/or symptoms associated therewith. It will be appreciated that, although not precluded, treating a disease, disorder, or condition does not require that the disease, disorder, pathology, condition, or symptoms associated therewith, be completely eliminated.

By “transformed cell” is meant a cell into which (or into an ancestor of which) has been introduced, by means of recombinant DNA techniques, a polynucleotide molecule encoding (as used herein) a polypeptide as described herein.

The terms “transfection”, “transduction”, “transfecting” or “transducing” can be used interchangeably and are defined as a process of introducing a nucleic acid molecule or a protein to a cell. Nucleic acids are introduced to a cell using non-viral or viral-based methods. The nucleic acid molecules may be gene sequences encoding complete proteins or functional portions thereof. Non-viral methods of transfection include any appropriate transfection method that does not use viral DNA or viral particles as a delivery system to introduce the nucleic acid molecule into the cell. Exemplary non-viral transfection methods include calcium phosphate transfection, liposomal transfection, nucleofection, sonoporation, transfection through heat shock, magnetofection and electroporation. In some embodiments, the nucleic acid molecules are introduced into a cell using electroporation following standard procedures well known in the art. For viral-based methods of transfection any useful viral vector may be used in the methods described herein. Examples for viral vectors include, but are not limited to retroviral, adenoviral, lentiviral and adeno-associated viral vectors. In some embodiments, the nucleic acid molecules are introduced into a cell using a retroviral vector following standard procedures well known in the art. The terms “transfection” or “transduction” also refer to introducing proteins into a cell from the external environment. Typically, transduction or transfection of a protein relies on attachment of a peptide or protein capable of crossing the cell membrane to the protein of interest. See, e.g., Ford et al. (2001) Gene Therapy 8:1-4 and Prochiantz (2007) Nat. Methods 4:119-20.

As used herein, the terms “treat,” treating,” “treatment,” and the like refer to reducing or ameliorating a disorder and/or symptoms associated therewith. It will be appreciated that, although not precluded, treating a disorder or condition does not require that the disorder, condition or symptoms associated therewith be completely eliminated.

An “effective amount” is an amount sufficient to accomplish a stated purpose (e.g. achieve the effect for which it is administered, treat a disease, reduce enzyme activity, reduce one or more symptoms of a disease or condition, reduce viral replication in a cell). An example of an “effective amount” is an amount sufficient to contribute to the treatment, prevention, or reduction of a symptom or symptoms of a disease, which could also be referred to as a “therapeutically effective amount.” A “reduction” of a symptom or symptoms (and grammatical equivalents of this phrase) means decreasing of the severity or frequency of the symptom(s), or elimination of the symptom(s). A “prophylactically effective amount” of a drug is an amount of a drug that, when administered to a subject, will have the intended prophylactic effect, e.g., preventing or delaying the onset (or reoccurrence) of an injury, disease, pathology or condition, or reducing the likelihood of the onset (or reoccurrence) of an injury, disease, pathology, or condition, or their symptoms. The full prophylactic effect does not necessarily occur by administration of one dose, and may occur only after administration of a series of doses. Thus, a prophylactically effective amount may be administered in one or more administrations. An “activity decreasing amount,” as used herein, refers to an amount of antagonist required to decrease the activity of an enzyme or protein (e.g. Tat, Rev) relative to the absence of the antagonist. A “function disrupting amount,” as used herein, refers to the amount of antagonist required to disrupt the function of an enzyme or protein relative to the absence of the antagonist. The exact amounts will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Pickar, Dosage Calculations (1999); and Remington: The Science and Practice of Pharmacy, 20th Edition, 2003, Gennaro, Ed., Lippincott, Williams & Wilkins).

“Patient,” “subject” or “subject in need thereof” refers to a living organism or individual suffering from, afflicted with, having, at risk for, or susceptible or prone to, a disease, pathology, disorder, or condition that can be treated by using the products, compositions and methods provided herein. The term does not necessarily indicate that the subject has been diagnosed with a particular disease, but typically refers to an individual under medical supervision. Non-limiting examples include humans, other mammals, bovines, rats, mice, dogs, monkeys, goat, sheep, cows, deer, as well as other non-mammalian animals. In some embodiments, a patient or subject is human.

The terms “ultrasonic wave”, “acoustical energy”, “acoustic wave”, or “ultrasound” are used interchangeably herein to refer to the disturbance in a material corresponding to the mechanical transfer or mechanical transduction of energy through the material. In various embodiments, the disturbance is a vibration of the materials' components. In some embodiments, the material is a volume of liquid, a cell, a cell membrane, a tissue, or an organ.

Unless specifically stated or obvious from context, as used herein, the term “or” is understood to be inclusive. Unless specifically stated or obvious from context, as used herein, the terms “a”, “an”, and “the” are understood to be singular or plural.

Unless specifically stated or obvious from context, as used herein, the term “about” is understood as within a range of normal tolerance in the art, for example within 2 standard deviations of the mean. About can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein are modified by the term “about.”

The recitation of a listing of chemical groups in any definition of a variable herein includes definitions of that variable as any single group or combination of listed groups. The recitation of an embodiment for a variable or for an aspect herein includes that embodiment as any single embodiment or in combination with any other embodiments or portions thereof as described herein.

Any compositions or methods and embodiments thereof as provided herein can be combined with one or more of any of the other compositions, methods and embodiments thereof as provided herein.

### BRIEF DESCRIPTION OF THE DRAWINGS

FIGS. 1A-1D are images, a scanning electron micrograph, a plot, and a bar graph relating to identification of putative mechanosensory channels in the Venus flytrap trigger hair. FIG. 1A presents representative images of a soil-grown Venus flytrap clone (left), Venus flytrap leaf (center), and single trigger hair (right). Black arrowheads in the center image indicate trigger hairs on leaf. FIG. 1B presents a scanning electron micrograph of a trigger hair. Cells of the lever (L), indentation zone (In) and podium (P) are indicated. Also seen are the digestive glands on the floor of the lobe. FIG. 1C presents a plot showing fold-enrichment of protein-coding genes of >100 amino acids in length (black circles) in the trigger hair relative to the trap. FLYC1, FLYC2 and OSCA are as labeled “CPM,” i.e., counts per million of mapped sequencing reads. FIG. 1D presents a bar graph showing average Fragments Per Kilobase of transcript per Million mapped reads (FPKM) for FLYC1, FLYC2 and OSCA in traps and trigger hairs. Dots of the same shade of grey indicate paired biological replicates. **FDR<0.005.

FIGS. 2A-2E present phylogenetic trees and polypeptide topology maps relating to the molecular phylogenetic relationship of FLYCATCHER and OSCA proteins. FIG. 2A presents a phylogenetic tree presenting a phylogenetic analysis by maximum likelihood method to show the relationship between the conserved MscS domain of Escherichia coli MscS protein, Arabidopsis thaliana MSL proteins (MSL1-MSL10), and Dionaea muscipulaNenus flytrap (DmFLYC1 and DmFLYC2) and Drosera capensis/Cape sundew (DcFLYC1.1 and DcFLYC1.2) FLYCATCHER proteins. FIGS. 2B and 2C present polypeptide topology maps showing predicted topology for DmFLYC1 (FIG. 2B) and DmFLYC2 (FIG. 2C) proteins. FIG. 2D is a phylogenetic tree presenting a phylogenetic analysis by maximum likelihood method showing the relationship between Arabidopsis thaliana OSCA family proteins and Venus flytrap DmOSCA. FIG. 2E is a polypeptide topology map showing predicted topology for DmOSCA protein. In FIGS. 2A and 2D, bootstrap values >50 are shown; scale, substitutions per site. In FIGS. 2B, 2C and 2E, amino acid residues that are conserved with Arabidopsis MSL10, MSL5 and OSCA1.5, respectively, are indicated in dark circles, while residues similar in identity are indicated in lighter circles. The predicted pore domain for each protein is encircled by a black line.

FIGS. 3A-3E are images demonstrating FLYC1 mRNA localization in Venus flytrap trigger hairs. FIG. 3A is an image showing a toluidine blue-stained longitudinal section through the base of a trigger hair. Elongated sensory cells are visible at the indentation zone (arrowheads). FIG. 3B presents max projection images through a longitudinal section after fluorescent in situ hybridization. (Top) FLYC1 transcript and DAPI at low (left) and high (right) magnification of the indentation zone. The FLYC1 transcript puncta was localized in sensory cells. (Bottom) No signal was observed when using FLYC1-sense probes. High background fluorescence is observed in all channels. FIG. 3C presents an image showing toluidine blue-stained transverse section through the indentation zone. The cells forming the outer ring are presumed to be the mechanosensors. FIG. 3D presents max projection images through a transverse section after fluorescent in situ hybridization. FLYC1 transcript (red) at low (left) and high (right) magnification. The FLYC1 transcript puncta was localized predominately in the sensory cells of the indentation zone. FIG. 3E presents max projection images through a transverse section of the trigger hair lever. No transcript was observed. Scale bars, μm.

FIGS. 4A-4D are a collection of plots, schematics, and a bar graph demonstrating that FLYC1 induces stretch-activated currents. FIG. 4A provides a schematic of the patch clamp method and plots showing representative traces of stretch-activated currents recorded from FLYC1 expressing HEK-P1KO cells in the cell-attached patch clamp configuration at −80 mV membrane potential. Stimulus trace illustrated above the current trace. Left, currents in response to graded negative pressure steps from 0 to 100 mmHg (Δ 10 mmHg). Right, current in response to single pulse of −70 mmHg pressure. FIG. 4B is a bar graph showing quantification of maximal current response from cells transfected with mock (N=7), mouse PIEZO1 (N=5), or FLYC1 plasmid (N=10). p=0.0251 (Mock vs. PIEZO1); p=0.0070 (Mock vs. FLYC1); Dunn's multiple comparison test. FIG. 4C (left) presents plots showing representative single channel traces in response to stretch at the indicated membrane potential. FIG. 4C (right) presents plots showing average I-V relationship of stretch-activated single channel currents from FLYC1 transfected cells. FIG. 4D (left) presents plots showing representative stretch-activated single channel currents recorded from excised inside-out patch configuration in asymmetrical NaCl solution at the indicated membrane potential. FIG. 4D (right) presents a plot showing average I-V of stretch-activated single channel currents in asymmetrical NaCl solution (Eᵣₑᵥ: −30.0±1.4 mV (N=7)).

FIGS. 5A-5E are images and a bar graph demonstrating that DcFLYC1.1 and DcFLYC1.2 localize to touch-sensitive structures of Drosera. FIG. 5A is an image of the Cape sundew leaf showing tentacle projections with mucilage secretions. FIG. 5B is an image of tentacle bending in response to insect (house fly) touch. Arrowheads mark examples of tentacles that have bent inward. FIG. 5C is a bar graph showing relative expression of DcFLYC1.1 and DcFLYC1.2 in sundew tentacles versus tentacle-less leaves by qRT-PCR. **p<0.005, moderated t test, results from four biological replicates of each tissue type. FIG. 5D presents an image showing toluidine blue-stained longitudinal section through the head and upper neck of a Cape sundew tentacle. The head is composed of xylem (X), an endodermis-like layer (E), and secretory cells (S). FIG. 5E presents Max projection images, at low (left) and high magnification (right), showing collective localization of DcFLYC1.1 and DcFLYC1.2 mRNAs to the outer secretory cells of the tentacle head (top). Arrows indicate example secretory cells with DcFLYC puncta. No signal above background was observed in the leaf (bottom). In FIGS. 5A, 5B, 5D, and 5E, scale bars represent μm.

FIGS. 6A and 6B are schematics presenting models of touch-induced movements in carnivorous Droseraceae plants. FIG. 6A presents a schematic model for the Venus flytrap. Not wishing to be bound by theory, mechanical stimulation of the trigger hair by a prey animal causes bending at the indentation zone sensory cells (1), leading to mechanically induced activation of mechanosensitive (MS) channels and chloride efflux (2). This triggers an action potential that propagates from the base of the sensory cells to cells of the podium via plasmodesmata (3). Propagation of action potentials through the lobe of the leaf results in trap closure. FIG. 6B presents a schematic model for sundew tentacles. Not wishing to be bound by theory, the pulling of mucilage could lead to activation of DcFLYC1.1 and DcFLYC1.2 proteins in the outer cell layer of tentacle heads, triggering a propagating action potential down the tentacle stalk.

FIGS. 7A and 7B are an image and a diagram relating to a Venus flytrap clonal propagation system. FIG. 7A is an image presenting an example of clonal Venus flytraps growing in tissue culture (10 cm plate) using methods adapted from Jang and Park. See Jang, G. W., Kim, K. S., & Park, R. D. (2003) “Micropropagation of Venus fly trap by shoot culture” Plant Cell Tissue and Organ Culture, 72(1), 95-98. FIG. 7B presents a diagram depicting the method of propagation. Rosettes were separated by splitting the rhizome. Plants were then transferred to fresh sterile growth medium for further propagation in culture or transferred to soil and ‘hardened’ for at least 2-3 months prior to experiments.

FIGS. 8A and 8B present images showing autofluorescence and EF1α transcript expression in Venus flytrap. FIG. 8A presents images of an unprocessed Venus flytrap trigger hair in different channels to depict autofluorescence from the plant cuticle. Images were taken with the same settings as tissue subjected to in situ hybridization. FIG. 8B presents images of a longitudinal section of a trigger hair and trap with smFISH against the housekeeping gene EF1α and FLYC1 as well as DAPI. White arrowheads indicate examples of EF1α expression in various cells throughout the tissue. Scale bars, μm.

FIGS. 9A and 9B present images showing FLYC2 and DmOSCA transcript expression in a trigger hair. Max projection through longitudinal sections after fluorescent in situ hybridization of (FIG. 9A) DmFLYC2 transcript and (FIG. 9B) DmOSCA transcript at low (left) and high resolution (center and right). No transcript was observed for DmFLYC2, whereas OSCA was observed mostly in the sensory cells. Scale bars, μm.

FIG. 10 is a bar graph relating to DmFLYC2 and DmOSCA functionality. Macroscopic stretch-activated currents were recorded from HEK P1-KO cells transfected with plasmids containing polynucleotides encoding each of the polypeptides identified along the x-axis, namely, Mock (N=7), MmPiezol (N=5), AtMSL10 (N=9), DmFLYC1 (N=10), DmFLCY2 (N=10), AtOSCA1.5 (N=4), and DmOSCA (N=12) plasmids.

FIG. 11 is a plot and a schematic. FIG. 11 presents a plot showing chloride permeability in DmFLYC1. Average I-V (N=5) of DmFLYC1 stretch-activated currents recorded in cell attached patch clamp configuration. The recording pipette was composed of 100 mM Calcium-gluconate. The schematic depicts the patch clamp technique to record stretch-activated currents.

FIGS. 12A-12D present a sequence alignment, protein structural images, and a plot relating to the sequence of a putative pore-forming helix in FLYC1 is compatible with MscS-like channel structure. FIG. 12A presents a sequence alignment of the putative pore helix of Venus flytrap FLYC1 and Drosera DcFLYC1.1 and DcFLYC1.2 proteins with MSL10 and MscS. Nonpolar and polar residues are shown as light grey and ionizable residues are shown in dark grey. A glycine predicted to localize at a central bend in the helix is shown within a grey box. The sequences presented in FIG. 12A are as follows:

FLYC1 (D. muscipula)
(SEQ ID NO: 1)
ATTKLIVLLSSQLVVAAFIFGNTCKTIFEAIIFVFVMHP
FLYC1.1 (D. capensis)
(SEQ ID NO: 2)
MTTKVLLFFSSQLLVAVFVFGNTCKTIFEAIIFVFVMHP
FLYC1.2 (D. capensis)
(SEQ ID NO: 3)
MTTKVLLFFTSQLLVAVFVFGNTCKTIFEAIIFVFVMHP
McsS (E. coli)
(SEQ ID NO: 4)
QTASVIAVLGAAGLAVGLALQGSLSNLAAGVLLVMFRPF

FIG. 12B is an image presenting modeled heptameric organization. The sequence of Venus flytrap FLYC1 was threaded on the inner helix of heptameric MscS in a closed conformation (PDB 2OAU), and minimized using the Rosetta energy function while imposing C7 symmetry. Predicted intersubunit hydrogen bonds between serines of pore segments are shown, while a ring of phenylalanines (F572) constrict the pore. Helices TM6a, forming the central pore, and amphipathic helix TM6b are indicated for one pore segment. FIG. 12C is an image of a cross section through the protein surface colored by electrostatic potential, showing an uncharged (light grey) pore, with positive charge (darker grey) above and below the pore. FIG. 12D is a plot presenting average I-V of stretch-activated single channel currents from FLYC1 (N=7) and FLYC1(K579E) (N=4) in asymmetrical NaCl solution. FLYC1 data are the same as that described in FIG. 3D.

FIGS. 13A and 13B are scanning electron microscopy images showing variation in Cape sundew tentacles. FIG. 13A is a scanning electron microscopy (SEM) image showing mucilage-producing tentacles of different lengths on the Cape sundew leaf blade. An asterisk (*) marks the staging needle. Tentacles increase in length towards the leaf edge. FIG. 13B presents a composite image of three SEM micrographs showing tentacle bending around a D. melanogaster fly. Scale bars, 200 μm.

FIG. 14 is an image showing unique cell morphology of Cape sundew sensory/excretory cells by presenting toluidine blue-stained longitudinal section through the head of a Cape sundew tentacle. E, endodermis-like cells; S, outer two layers of secretory cells. Arrows mark the unique cell wall buttresses of a single outer secretory cell, around which membrane crenellations occur.

FIG. 15 presents a graph showing Drosera FLYC functionality. Macroscopic stretch-activated currents recorded from HEKP1-KO cells transfected with Mock (N=7), DmFLYC1 (N=10), DcFLCY1.1 (N=7), DcFLYC1.2 (N=10), and DcFLYC1.1/1.2 (N=11) plasmids.

FIGS. 16A and 16B present images and a plot demonstrating that leaves of a Venus fly trap respond to ultrasound stimuli. FIG. 16A presents images showing Venus fly trap leaves open (top) and closed (bottom) after ultrasound stimuli. FIG. 16B presents a plot showing the number of 100 msec pulses needed to close the trap at different ultrasound intensities. n>30 for each condition. Averages and standard deviations are shown.

FIGS. 17A and 17B present plots demonstrating that FLYC1 suppresses INS1 activity upon application of ultrasound. The plots show ratio (y-axis has units of deltaF/F) of changes in GCaMP fluorescence to baseline in INS1 controls (FIG. 17A) and in INS1-FLYC1 cells (FIG. 17B). 100 msec pulses of ultrasound are represented by vertical gray bands. n>17. The x-axis is labeled “Frame”, which has units of 0.1 seconds, and the y-axis is labeled “deltaF/F”.

FIGS. 18A and 18B provide graphs demonstrating that FLYC1 suppresses INS1 activity upon ultrasound. The graph shown in FIG. 18A demonstrates that INS1 cells show a dose-dependent response to KCl in the FLIPR assay. The graph shown in FIG. 18B demonstrates that FLYC1 expression suppresses INS1 response to KCl upon ultrasound stimulation. n=12 wells each, * p<0.05 t-test with Bonferroni correction.

FIG. 19 presents patch-clamp electrophysiology traces resulting from the monitoring of sono-silencing channels. The traces provide examples of excitatory HEK cells expressing FLYC1 stimulated with different durations of ultrasound stimuli from a 6.91 MHz transducer.

FIGS. 20A and 20B show dot plots demonstrating the FLYC1 suppresses ultrasound-evoked hsTRPA1 calcium events. Shown is the ratio of change in GCaMP fluorescence relative to baseline in HEK cells expressing hsTRPA1+dTom controls or hsTRPA1+FLYC1 upon ultrasound stimulation (FIG. 20A) or exposure to AITC chemical agonist (30 μM concentration), (FIG. 20B). n>60 for each condition. ** indicates p<0.01 by logistic regression.

FIGS. 21A and 21B show representative traces and plots demonstrating that the R334E variant exhibits different activation kinetics. FIG. 21A presents example traces of stretch-activated current from HEK-P1KO cells expressing wildtype (WT), (top trace) and R334E (bottom trace) in cell attached configuration at −80 mV membrane potential in response to −70 mmHg pipette pressure. FIG. 21B (left plot) shows current time and inactivation time (right plot) time for the cells as described in FIG. 21A. n=6. ** indicates p<0.01 Dunn's multiple comparison test.

### DETAILED DESCRIPTION OF THE EMBODIMENTS

Provided and featured herein are products and compositions featuring mechanosensory polypeptides and polynucleotides, methods for expressing such polypeptides and polynucleotides in a cell type of interest, and methods for inducing the activation of the mechanosensory polypeptide in neuronal cells (e.g., neurons) and other cell types using ultrasound.

The aspects and embodiments as described herein are based, at least in part, on the discovery that specific proteins, e.g., mechanosensory proteins, derived from plants confer sensitivity to ultrasound when transduced or transfected into target cells and expressed as heterologous proteins on the target cell surface. The Examples provided and described herein demonstrate that such mechanosensory proteins have a sensitivity to ultrasound. Not wishing to be bound by theory, ultrasound can generate a mechanical deflection in the focal zone that leads to activation of the expressed mechanosensory protein. In embodiments described and exemplified herein, the mechanosensory proteins can be used non-invasively to control cells.

In particular, as described and exemplified herein, the Venus flytrap protein FLYCATCHER1 (FLYC1) was identified as a polypeptide (protein) capable of inducing chloride-permeable stretch-activated and/or ultrasound-activated currents in naïve cells transfected or transduced with a polynucleotide encoding the polypeptide (protein). As described and demonstrated herein, FLYC1 is a chloride-selective inhibitory mechanosensitive channel protein in mammalian cells. FLYC1 was expressed in excitable HEK cells and spontaneously active insulinoma cells (INS-1) and the protein was able to inhibit an activity in an ultrasound-dependent manner in both types of cell lines. Not wishing to be bound by theory, the FLYC1 polypeptide was determined to be a mechanosensitive ion channel likely allowing the Venus flytrap to sense touch. The products, compositions and methods described herein allow for precision targeting of cell inhibition and for inhibiting or silencing the activity of cells upon ultrasound stimulation.

As described and exemplified herein, members of the FLYC and OSCA families of ion channels were identified as candidate mechanosensors in rapid touch sensation in carnivorous plants. Members of both ion channel families were shown to have enriched expression in the sensory trigger hairs of Venus flytrap. Among these, the FLYC1 gene was identified as a prime candidate as its mRNA is highly expressed/enriched in the putative mechanosensory cells that initiate transduction of the touch-induced signal. The encoded polypeptide, FLYC1, formed a mechanically activated ion channel with properties that would facilitate generation of action potentials in sensory cells. In addition, expression of FLYC genes was remarkably conserved in two morphologically disparate touch-sensitive structures from different genera in the Droseraceae family.

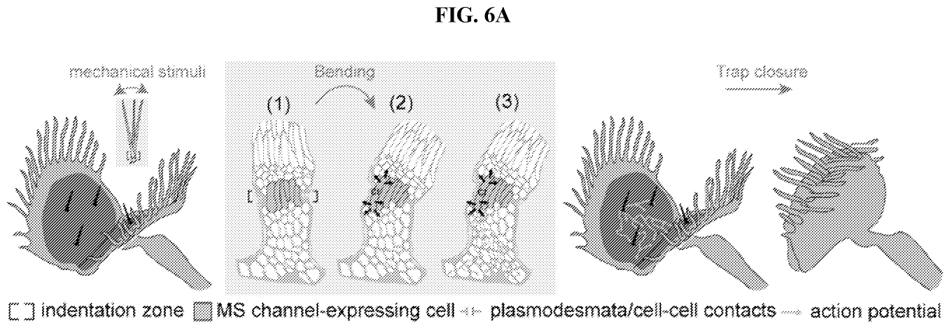

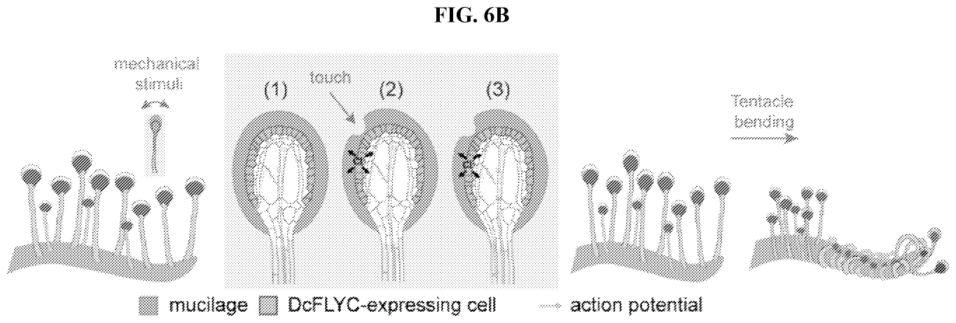

As described and demonstrated in the examples herein, robust macroscopic, as well as single channel stretch-activated currents, were detected in FLYC1-expressing cells in a mammalian cell-expression system, and residues important for channel properties were identified, supporting the finding that FLYC1 formed mechanosensitive ion channels. Not intending to be bound by theory, a chloride-permeable channel like FLYC1 could contribute to membrane depolarization in plant cells. This is consistent with the finding that increasing concentrations of extracellular chloride ions was shown previously to reduce or abolish the electrical response of Venus flytrap sensory cells to a mechanical stimulus. FLYC1-mediated depolarization, likely in combination with FLYC2 and OSCA (a mechanosensitive calcium permeable ion channel), may initiate action potentials that propagate into the leaf through plasmodesmata clustered on the basal side of the sensory cells, eliciting trap closure (FIG. 6A). A similar chain of events would occur in the sundew tentacle: prey contact with the tentacle results in DcFLYC-induced action potentials that may propagate through plasmodesmata along the outer cell layers of the tentacle mechanosensory head and stalk, evoking tentacle bending (FIG. 6B). In an embodiment, in plant cells, FLYC1 is likely a chloride-selective excitatory mechanosensitive channel protein in plant cells. The findings described and exemplified herein are consistent with FLYC1 being identified as a bona fide sensor of touch. In various embodiments, the FLYC1 polypeptide may be used to modulate an activity in a cell when expressed as a heterologous polypeptide in the cell.

In various embodiments, the described heterologous mechanosensory proteins described herein can be used to render neurons, cardiac muscle, urinary bladder tissues, T-cells, or beta-cells responsive to ultrasound. In some embodiments, the mechanosensory proteins can be used to render sensitive to ultrasound any cell type that is sensitive to a rapid change in cation (e.g., calcium, potassium, sodium) or anion (e.g., chloride, fluoride, bromide, or iodide) concentration. The mechanosensory proteins can be used to alter cellular functions in vivo, in vitro, or ex vivo. In addition, the mechanosensory proteins can be used to alter cell function of cells expressing these proteins in cell culture.

Accordingly, provided and described are polynucleotides encoding a mechanosensory polypeptide, expression vectors comprising such polynucleotides, cells expressing a heterologous mechanosensory polypeptide, cells expressing a heterologous recombinant mechanosensory polypeptide, and methods for stimulating such cells with ultrasound.

### Ultrasound

Ultrasound is well suited for stimulating neuron populations as it focuses easily through intact thin bone and deep tissue (K. Hynynen and F. A. Jolesz, Ultrasound Med Biol 24 (2), 275 (1998)) to volumes of just a few cubic millimeters (G. T. Clement and K. Hynynen, Phys Med Biol 47 (8), 1219 (2002)). The non-invasive nature of ultrasound stimulation is particularly significant for manipulating vertebrate neurons including those in humans, as it eliminates the need for invasive techniques, such as surgery, to insert light fibers (required for some current optogenetic methods). Also, the small focal volume of the ultrasound wave compares well with light that is scattered by multiple layers of brain tissue (S. I. Al-Juboori et al., PLoS ONE 8 (7), e67626 (2013)). Moreover, ultrasound has been previously used to manipulate deep nerve structures in human hands and reduce chronic pain (W. D. O'Brien, Jr., Prog Biophys Mol Biol 93 (1-3), 212 (2007); L. R. Gavrilov et al., Prog Brain Res 43, 279 (1976)). As described herein, novel, non-invasive compositions for the heterologous expression of mechanosensory polypeptides in cells are provided, and methods to stimulate or modulate the activity or function of cells expressing heterologous mechanosensory polypeptides using low-intensity ultrasound stimulation are provided. In an embodiment, the cells that express the heterologous mechanosensory polypeptides are neuronal cells, and the methods involve neuromodulation by the non-invasive use of ultrasound stimulation, in particular, low-intensity ultrasound.

### Cells and Cellular Compositions Comprising Recombinant Mechanosensory Polypeptides

Provided are cells comprising a heterologous nucleic acid molecule encoding a mechanosensory polypeptide (e.g., FLYC1). Such mechanosensory polypeptides are heterologously or exogenously expressed in a cell type of interest. In an embodiment, the cell type of interest expresses a heterologous mechanosensory polypeptide as described herein and is sensitive to a rapid change in anion (e.g., chloride, bromide, fluoride, or iodide) or cation (e.g., calcium, potassium, sodium) concentration associated with mechanosensory modulation or inhibition of an activity of the cell by ultrasound. In an embodiment, the cell type is a cardiac muscle cell comprising a mechanosensory polynucleotide under the control of a promoter suitable for expression in a cardiac cell (e.g., NCX1 promoter). In an embodiment, the cell type is a muscle cell comprising a mechanosensory polynucleotide under the control of a promoter suitable for expression in a muscle cell, e.g., myoD promoter. In another embodiment, the cell type is an insulin secreting cell (e.g., a beta (β) islet cell) comprising a mechanosensory polynucleotide under the control of a promoter suitable for expression in an insulin-secreting cell, e.g., Pdx1 promoter. In another embodiment, the cell type is an adipocyte comprising a mechanosensory polynucleotide under the control of a promoter suitable for expression in an adipocyte (e.g., iaP2). In another embodiment, the cell type is a neuronal cell type (neuron) comprising a mechanosensory polynucleotide under the control of a promoter suitable for expression in a neuronal cell. In an embodiment and by way of nonlimiting example, the neuronal cell may be a neuron in the central nervous or peripheral nervous system, a neuron in the brain or spinal cord, a motor neuron, a sensory neuron, an interneuron, or an Agouti-Related Protein-expression positive (AGRP-^{+ve}) neuron. By way of nonlimiting examples, a nestin or Tuj 1 promoter is generally suitable for expression of the mechanosensory polynucleotide in a neuron; an H2b promoter is suitable for expression of the mechanosensory polynucleotide in a motor neuron; an Islet 1 promoter is suitable for expression of the mechanosensory polynucleotide in an interneuron; and an OMP promoter, T1R, T2R promoter, rhodopsin promoter, or Trp channel promoter is suitable for expression of the mechanosensory polynucleotide in a sensory neuron. In some embodiments, the cell is a plant cell. In some embodiments, the cell can be an cell of the immune system, e.g., a T cell, B cell, monocyte, macrophage, natural killer (NK) cell. Such cells may be cells in vitro, ex vivo, or in vivo. In particular embodiments, the cells express a mechanosensory polypeptide that is sensitive to ultrasound. In particular embodiments, the mechanosensory polypeptide is a DmFLYC1, DmFLYC2, DmOSCA, DcFLYC1.1, or DcFLYC1.2 polypeptide, or a functional portion, isoform, ortholog, or homolog thereof. In another embodiment, the mechanosensory polypeptide, e.g., a DmFLYC1, DmFLYC2, DmOSCA, DcFLYC1.1, or DcFLYC1.2 polypeptide, or a functional portion, isoform, ortholog, or homolog thereof, or the polynucleotide encoding the mechanosensory polypeptide and the like, is codon-optimized for expression in a mammalian cell or in a plant cell. In another embodiment, the mechanosensory polypeptide, e.g., a DmFLYC1, DmFLYC2, DmOSCA, DcFLYC1.1, or DcFLYC1.2, or a functional portion, isoform, ortholog, or homolog thereof, or the polynucleotide encoding the mechanosensory polypeptide and the like, is codon-optimized for expression in a mammalian cell or a human cell.

### Expression of Recombinant Mechanosensory Polypeptides

In one approach, a cell of interest (e.g., a neuron, such as a motor neuron, sensory neuron, neuron of the central or peripheral nervous system, neuronal cell lines, or a plant cell) is genetically or recombinantly engineered to express a heterologous mechanosensory polynucleotide whose expression renders the cell responsive to ultrasound stimulation. Ultrasound stimulation of such cells induces cation or anion influx or efflux. The molecular techniques involved in genetically or recombinantly engineering cells to express heterologous polypeptides are well known to and routinely practiced by those having skill in the art.

The mechanosensory polypeptide may be constitutively expressed or its expression may be regulated by an inducible promoter or other control mechanism where conditions necessitate highly controlled regulation or timing of the expression of a mechanosensory polypeptide. For example, heterologous DNA encoding a mechanosensory polypeptide gene to be expressed is inserted in one or more pre-selected DNA sequences. This can be accomplished by homologous recombination or by viral integration into the host cell genome. The desired gene sequence can also be incorporated into a cell, particularly into its nucleus, using a plasmid expression vector and a nuclear localization sequence. Methods for directing polynucleotides to the nucleus have been described in the art. The genetic material can be introduced using promoters that will allow for the gene of interest to be positively or negatively induced using certain chemicals/drugs, to be eliminated following administration of a given drug/chemical, or can be tagged to allow induction by chemicals, or expression in specific cell compartments.

Calcium phosphate transfection can be used to introduce plasmid DNA containing a target gene or polynucleotide into cells and is a standard method of DNA transfer to those of skill in the art. DEAE-dextran transfection, which is also known to those of skill in the art, may be preferred over calcium phosphate transfection where transient transfection is desired, as it is often more efficient. Since the cells as described herein are isolated cells, microinjection can be particularly effective for transferring genetic material into the cells. This method is advantageous because it provides delivery of the desired genetic material directly to the nucleus, avoiding both cytoplasmic and lysosomal degradation of the injected polynucleotide. Cells can also be genetically modified using electroporation.

Liposomal delivery of DNA or RNA to genetically modify the cells can be performed using cationic liposomes, which form a stable complex with the polynucleotide. For stabilization of the liposome complex, dioleoyl phosphatidylethanolamine (DOPE) or dioleoyl phosphatidylcholine (DOPQ) can be added. Commercially available reagents for liposomal transfer include Lipofectin (Life Technologies). Lipofectin, for example, is a mixture of the cationic lipid N-[1-(2, 3-dioleyloxy)propyl]-N—N—N-trimethyl ammonia chloride and DOPE. Liposomes can carry larger pieces of DNA, can generally protect the polynucleotide from degradation, and can be targeted to specific cells or tissues. Cationic lipid-mediated gene transfer efficiency can be enhanced by incorporating purified viral or cellular envelope components, such as the purified G glycoprotein of the vesicular stomatitis virus envelope (VSV-G). Gene transfer techniques which have been shown effective for delivery of DNA into primary and established mammalian cell lines using lipopolyamine-coated DNA can be used to introduce target DNA into the de-differentiated cells or reprogrammed cells described herein.

Naked plasmid DNA can be injected directly into a tissue comprising cells of interest. Microprojectile gene transfer can also be used to transfer genes into cells either in vitro or in vivo. The basic procedure for microprojectile gene transfer was described by J. Wolff in Gene Therapeutics (1994), page 195. Similarly, microparticle injection techniques have been described previously, and methods are known to those of skill in the art. Signal peptides can be also attached to plasmid DNA to direct the DNA to the nucleus for more efficient expression.

Viral vectors are used to genetically alter cells as provided and described herein and their progeny. Viral vectors are used, as are the physical methods previously described, to deliver one or more polynucleotide sequences encoding the mechanosensory polypeptides, for example, into the cells. Viral vectors and methods for using them to deliver or introduce polynucleotides such as DNA or RNA, to cells are well known to those of skill in the art. Examples of viral vectors that can be used in the delivery of polynucleotides into the cells as provided and described herein include, but are not limited to, adenoviral vectors, adeno-associated viral vectors (AAV), retroviral vectors (including lentiviral vectors), alpha viral vectors (e. g., Sindbis vectors), and herpes virus vectors. In embodiments, the viral vectors are recombinant viral vectors.

### Targeted Cell Types

Mechanosensory polypeptides can be heterologously expressed in virtually any eukaryotic or prokaryotic cell of interest. In one embodiment, the cell (or target cell) is a bacterial cell or a pathogenic cell type. In another embodiment, the cell (or target cell) is a mammalian cell, such as an adipocyte, muscle cell, cardiac muscle cell, immune cell, insulin secreting cell (e.g., beta (B) islet cell), or neuron (e.g., a motor neuron, a sensory neuron, a neuron of the central nervous system (e.g., a neuron in the brain or in a region of the brain, e.g., the hypothalamus), a neuron of the peripheral nervous system, an interneuronal cell, and neuronal cell lines or populations). In some embodiments, the cell is a plant cell. In some embodiments, the plant cell is not a Dionaea muscipula or a Drosera capensis cell.

Methods of Modulating Activity and/or Function of a Cell

The methods provided herein are, inter alia, useful for the modulation (e.g. increase, decrease, silencing, or inhibition) of an activity and/or function of cells that express the mechanosensory polypeptides. In particular, ultrasound stimulation of such cells induces cation or anion influx or efflux, thereby altering cell activity and/or function. Altering the intensity of the ultrasound modulates the extent of alteration in the function and/or activity.

The terms “neuron,” “neural cell,” or “neuronal cell” are used interchangeably herein and refer to a cell of the brain or nervous system, such as the central nervous system (CNS) or the peripheral nervous system (PNS). Non-limiting examples of neural or neuronal cells include neurons, interneurons, glial cells, astrocytes, oligodendrocytes and microglia cells. Where a neural cell is stimulated, a function or activity (e.g., excitability) of the neural cell is modulated by modulating, for example, the expression or activity of a given gene or protein (e.g., a mechanosensory polypeptide) within the neural cell. The change in expression or activity may be 10%, 20%, 30%, 40%, 50%, 60%, 70%, 80%, 90% or more in comparison to a control (e.g., unstimulated cell). In certain instances, expression or activity is 1.5-fold, 2-fold, 3-fold, 4-fold, 5-fold, 10-fold or higher or lower than the expression or activity in the absence of stimulation. In certain instances, expression or activity is 1.5-fold, 2-fold, 3-fold, 4-fold, 5-fold, 10-fold or lower than the expression or activity in the absence of stimulation. The neural cell may be stimulated by applying an ultrasonic wave to the neural cell.

The term “applying” as provided herein is used in accordance with its plain ordinary meaning and includes the terms contacting, introducing and exposing. An “ultrasonic wave” as provided herein is an oscillating sound pressure wave having a frequency greater than the upper limit of the human hearing range. Ultrasound (ultrasonic wave) is thus not separated from “normal” (audible) sound by differences in physical properties, only by the fact that humans cannot hear it. Although this limit varies from person to person, it is approximately 20 kilohertz (20,000 hertz) in healthy, young adults. Ultrasound (ultrasonic wave) devices operate with frequencies from 20 kHz up to several gigahertz. The methods provided herein use the energy of an ultrasonic wave to stimulate a neural cell expressing an exogenous mechanosensory protein. A mechanotransduction protein as provided herein refers to a cellular protein capable of converting a mechanical stimulus (e.g., sound, pressure, movement) into chemical activity. Cellular responses to mechanosensation or mechanotransduction are variable and give rise to a variety of changes and sensations. In some embodiments, the mechanosensory protein is a mechanically gated ion channel, which makes it possible for sound, pressure, or movement to cause a change in the excitability of a cell (e.g., a sensory neuron). The stimulation of a mechanosensory protein may cause mechanically sensitive ion channels to open and produce a transduction current that changes the membrane potential of a cell.

In one aspect, a method of stimulating a cell is provided. The method includes transfecting or transducing a cell with a recombinant vector or viral vector containing a nucleic acid sequence encoding an exogenous (heterologous) mechanosensory polypeptide, wherein the transfected or transduced cell expresses the mechanosensory polypeptide, in particular, in the cell membrane. An ultrasonic wave is applied to the transfected or transduced cell, thereby stimulating the cell. In some embodiments, the mechanosensory polypeptide is DmFLYC1, DmFLYC2, DmOSCA, DcFLYC1.1, DcFLYC1.2, or a homolog or ortholog thereof. In some embodiments, the mechanotransduction polypeptide is a mechanosensory polypeptide or a functional portion, homolog, or ortholog thereof. In some embodiments, ultrasound applied to a cell expressing a heterologous DmFLYC1, DmFLYC2, DmOSCA, DcFLYC1.1, DcFLYC1.2 mechanosensory polypeptide or a homolog or ortholog thereof results in a reduction, inhibition, or silencing of an activity or function in the cell. In some embodiments, the ultrasonic wave has a frequency of about 0.8 MHz to about 4 MHz. In some embodiments, the ultrasonic wave has a frequency of about 1 MHz to about 3 MHz. In some embodiments, the ultrasonic wave has a focal zone of about 1 cubic millimeter to about 1 cubic centimeter. In some embodiments, the ultrasonic wave has a frequency of about 385 KHz. In some embodiments, the ultrasonic wave has a frequency of about 10 MHz. In some embodiments, the ultrasonic wave has a frequency of about or of at least about 0.001 MHz, 0.01 MHz, 0.1 MHz, 0.2 MHz, 0.3 MHz, 0.4 MHz, 0.5 MHz, 1 MHz, 2 MHz, 3 MHz, 4 MHz, 5 MHz, 6 MHz, 7 MHz, 8 MHz, 9 MHz, 10 MHz, 11 MHz, 12 MHz, 13 MHz, 14 MHz, 15 MHz, 20 MHz, 30 MHz, 40 MHz, or 50 MHz. In some embodiments, the ultrasonic wave has a frequency of less than about 0.001 MHz, 0.01 MHz, 0.1 MHz, 0.2 MHz, 0.3 MHz, 0.4 MHz, 0.5 MHz, 1 MHz, 2 MHz, 3 MHz, 4 MHz, 5 MHz, 6 MHz, 7 MHz, 8 MHz, 9 MHz, 10 MHz, 11 MHz, 12 MHz, 13 MHz, 14 MHz, 15 MHz, 20 MHz, 30 MHz, 40 MHz, or 50 MHz. In some embodiments, the ultrasonic wave has a frequency of from about 0.2 MHz to about 20 MHz, from about 0.15 MHz to about 0.6 MHz, from about 0.3 MHz to about 0.4 MHz, from about 9 MHz to about 11 MHz, or of from about 5 MHz to about 20 MHz. In some embodiments, the ultrasonic wave has an intensity of less than 500 mW/cm².

In some embodiments, the ultrasonic wave has an intensity of from about or at least about 0.01 W/cm², 0.5 W/cm², 1 W/cm², 5 W/cm², 10 W/cm², 25 W/cm², 50 W/cm², 100 W/cm², 150 W/cm², 200 W/cm², 250 W/cm², 300 W/cm², or 400 W/cm². In some embodiments, the ultrasonic wave has an intensity of less than about 0.01 W/cm², 0.5 W/cm², 1 W/cm², 5 W/cm², 10 W/cm², 25 W/cm², 50 W/cm², 100 W/cm², 150 W/cm², 200 W/cm², 250 W/cm², 300 W/cm², or 400 W/cm². In some embodiments, the ultrasonic wave produces a peak negative pressure of from between 0.05 and 3 MPa within a targeted region.

In some embodiments, ultrasonic waves are administered to a cell or tissue in pulses or bursts. In some embodiments, the pulse repetition frequency for the pulses or bursts is from about 0.1 Hz to about 200 Hz, or from about 0.5 Hz to about 2 Hz. In some embodiments, the pulse repetition frequency is about 1 Hz. In various embodiments, the ultrasonic waves are administered with a duty cycle from about 0.005% to about 100%, from about 0.01% to about 50%, from about 0.1% to about 10%, or from about 0.5% to about 2%. In some embodiments, the duty cycle is about 1%. By “duty cycle” is meant the fraction of the time duration of a single on and off cycle of ultrasonic wave administration over which an ultrasonic wave is actively administered.

In some embodiments, the method further includes contacting a transfected or transduced cell, e.g., without limitation, a neural cell, with an ultrasound contrast agent prior to applying ultrasound. In various embodiments, the ultrasound contrast agent is a microbubble. In certain embodiments, the microbubble has a diameter of from about 0.1 μm, 0.2 μm, 0.3 μm, 0.4 μm, 0.5 μm, 0.75 μm, 1 μm, 1.5 μm, 2 μm, 3 μm, 4 μm, or 5 μm to about 6 μm, 7 μm, 8 μm, 9 μm, 10 μm, 50 μm, or 100 μm. In certain embodiments, the neural cell forms part of an organism. In some embodiments, the organism is a mammal (e.g., non-human primate, human, murine, bovine, ovine, rodent, camelid, feline, canine mammal).

### Generation of Acoustical Energy (Ultrasound)

Various devices may be used to generate an ultrasound wave, such as acoustic and ultrasonic emitters, transducers or piezoelectric transducers, composite transducers, micromachined ultrasound transducers (MUTs) including capacitive micromachined ultrasonic transducers (cMUTs), Micro-Electro-Mechanical Systems (MEMS), silicon on insulator MEMS (SOI MEMS). A device for generating ultrasound waves may be provided as single or multiple transducers or in array configurations. The ultrasound waves may be of any shape, and may be focused or unfocused. Focal spot size depends on probe active aperture diameter (A), wavelength (lambda) and focal length (F). The center deflection of a clamped circular plate under a uniform pressure can be found from the following equation for a circular membrane

$$P = \frac{{Eh}^{4}}{{R}^{3}} [ \frac{16 y}{3 ( 1 - {v}^{2} ) h} + \frac{( 7 - v ) {y}^{3}}{3 ( 1 - {v}^{2} ) {h}^{3}} + \frac{4 {R}^{2} σ y}{( 1 - v ) {Eh}^{3}} ]$$

where P is the uniform pressure applied on the membrane, R is the membrane radius, y is the center deflection, 6 is the intrinsic stress of the membrane material, E is the Young's modulus of the membrane material, and v is Poisson's ratio of the membrane material. This equation can be used to estimate the pressure of the ultrasound waves under a prescribed membrane deflection. Such emitters may be made atop a substrate. Multiple substrates may be combined to form a single applicator. Multiple applicators may be combined to form a single probe.

In some embodiments, an ultrasonic wave is generated by an opto-acoustic system or transducer, such as that described, for example, in U.S. Pat. No. 6,022,309 and U.S. Pat. Appl. Pub. No. 20050021013, the entire disclosures of which are incorporated herein by reference in their entirety for all purposes. In some embodiments, ultrasonic waves are generated by optical energy delivered by pulsed laser light, optionally guided through an optical fiber. In some embodiments, the optical fiber is disposed within a catheter. In some embodiments, optical energy is deposited in a water-based optical energy-absorbing fluid, e.g. saline, thrombolytic agent, blood or thrombus, and generates an acoustic impulse in the fluid through thermoelastic and/or thermodynamic mechanisms. By pulsing the laser at a repetition rate (e.g., from 10 Hz to 100 kHz) ultrasonic waves can be established locally in the medium. In some embodiments, a high repetition rate laser system is used to produce the optical energy. In various embodiments, the laser light has a pulse frequency within the range of from about 10 Hz to about 100 kHz, or in the range of from about 20 kHz to about 1,000 kHz, or in the range of from about 0.1 MHz to about 50 MHz, or from about 0.1 MHz to about 20 MHz, or from about 0.2 MHz to about 10 MHz, or from about 1 MHz to about 20 MHz, or a wavelength within the range of about 200 nm to about 5000 nm and an energy density within the range of about 0.01 J/cm² to about 4 J/cm². In one embodiment, the pulse frequency is within the range of about 5 kHz to about 25 kHz. In various embodiments, an optical fiber used to deliver optical energy to a fluid has a core diameter of 200 microns or of 100 microns or less.

Not wishing to be bound by theory, an absorbing fluid responds thermoelasticly to the deposition of the optical energy such that a region of high pressure is created in the fluid in a volume of a composition (e.g., a fluid, blood, a tissue, a cell, a composition comprising cells, etc.) heated by the optical energy. The boundary of the high pressure zone decays into a pattern of acoustic waves and a compression wave propagates away from the energy deposition region (diverging wave front) and a rarefaction wave propagates towards the center of the energy deposition region (converging wave front). When the rarefaction wave converges on the center of the initial deposition region, it creates a region of tensile stress that promotes the formation of a cloud of cavitation bubbles which coalesce to form a larger bubble. Eventually, the cavitation bubble collapses resulting in an expanding acoustic wave. Collapse and subsequent rebound of the cavitation bubble will generate acoustic waves in the surrounding fluid, which will carry off a portion of the energy of the cavity. The collapse and rebound processes take place on a time scale governed principally by the fluid density and the maximum size of the initial cavity. The first collapse and rebound will be followed by subsequent collapse and rebound events of diminishing intensity until the energy of the cavity is dissipated in the fluid. In some embodiments, subsequent laser pulses are delivered to repeat or continue this cycle and generate ultrasonic waves at a frequency or frequencies determined by the laser pulse frequency.

The pulsed laser energy source used as described herein is not limiting and can be based on, as non-limiting examples, a gaseous, liquid or solid state medium. Rare earth-doped solid state lasers, ruby lasers, alexandrite lasers, Nd:YAG (neodymium-doped yttrium aluminum garnet; Nd:Y₃Al₅O₁₂) lasers and Ho:YLF (neodymium-doped yttrium lithium fluoride) lasers. Any of these solid state lasers may incorporate non-linear frequency-doubling or frequency-tripling crystals to produce harmonics of the fundamental lasing wavelength. A solid state laser producing a coherent beam of ultraviolet radiation may be employed directly in connection with the compositions and methods as described herein or used in conjunction with a dye laser to produce an output beam which is tunable over a wide portion of the ultraviolet and visible spectrum.

In one aspect, an ultrasonic wave may be generated in a fluid by: (i) depositing laser energy in a volume of the fluid comparable to a dimension (e.g., diameter or a maximum dimension of an area over which laser energy is absorbed by the fluid) of an optical fiber used to deliver the laser energy and in a time scale of duration less than the acoustic transit time across the dimension (as controlled by, for example, choice of laser wavelength and/or absorbing fluid); (ii) controlling the laser energy such that the maximum size of a generated cavitation bubble is approximately the same as the fiber dimension; and (iii) pulsing the laser at a repetition rate such that multiple cycles of this process generate an acoustic radiation field in the fluid. Resonant operation may be achieved by synchronizing the laser pulse repetition rate with cavity lifetime. In some embodiments, operation leads to a fluid-based transducer that cycles at 1-100 kHz with a reciprocating displacement of 100-200 μm. In various embodiments, the acoustic waves are propagated into tissue or fluid surrounding the small volume of fluid.

In another aspect, an ultrasonic wave may be generated in a fluid by: (i) depositing laser energy in a small volume of fluid (as controlled by, for example, choice of laser wavelength and absorbing fluid) within a blood vessel or circumvented by a tissue; (ii) controlling the laser energy such that the maximum size of a vapor bubble generated is approximately the same as or less than the diameter of a blood vessel within which the vapor bubble is generated or the diameter defined by the circumventing tissue within which the vapor bubble is generated; and (iii) pulsing the laser energy at a repetition rate such that multiple cycles of the bubble generation and collapse process generates an acoustic waves in the fluid. In various embodiments, the acoustic waves are propagated into tissue or fluid surrounding the small volume of fluid.

### Methods of Treatment and Cellular Manipulation

In an aspect, non-invasive therapeutic methods are provided and described, in which the methods involve the use of sound waves (ultrasound) to activate mechanosensitive cellular transmembrane proteins (mechanoreceptors) that in turn excite, increase, silence, or inhibit cellular function, locally and/or downstream of the initial source of ultrasound application. In another aspect, a method of altering the function of a sensory unit that innervates a targeted tissue portion of an animal is provided. In another aspect, a method for manipulating the activity or function of a cell or tissue is provided, in particular, a mammalian cell or tissue, including a human cell or tissue, or a plant cell or tissue. In an embodiment, the plant is not Dionaea muscipula or Drosera capensis. The method involves expressing a heterologous polypeptide in a cell. The methods include expressing in a cell or tissue of a subject a therapeutically effective amount of an exogenous mechanosensory polypeptide (e.g., DmFLYC1, DmFLYC2, DmOSCA, DcFLYC1.1, and DcFLYC1.2) and applying ultrasound (an ultrasonic wave) to the subject, cell, or tissue, thereby resulting in a change in mechanosensory polypeptide conductance, e.g., cation or anion influx or efflux, in the cell or tissue. In an embodiment, the methods include administering or delivering to a cell or tissue of a subject a therapeutically effective amount of a recombinant nucleic acid, a vector, or a viral vector encoding an exogenous mechanosensory polypeptide (e.g., DmFLYC1, DmFLYC2, DmOSCA, DcFLYC1.1, and DcFLYC1.2) and applying ultrasound (an ultrasonic wave) to the cell or tissue, resulting in a change in mechanosensory polypeptide conductance, e.g., cation or anion influx or efflux. In an embodiment, the method is used to manipulate a tissue or cell ex vivo, in vivo, in situ, or ex situ. In an embodiment, the methods involve treating or ameliorating a disease or disorder by modulating, e.g., enhancing, increasing, silencing, or inhibiting cellular activity or function in a subject having the disease or disorder. In particular embodiments, the exogenous mechanosensory polypeptide is a mechanosensory polypeptide, namely, DmFLYC1, DmFLYC2, DmOSCA, DcFLYC1.1, DcFLYC1.2, an ortholog or homolog thereof, or a functional portion thereof.

In some embodiments, the method further includes administering to the subject, cell, or tissue an ultrasound contrast agent prior to the application of ultrasound to the subject, cell, or tissue. In some embodiments, the ultrasound contrast agent is a microbubble. In some embodiments, the microbubble has a diameter of from about 0.1 μm, 0.2 μm, 0.3 μm, 0.4 μm, 0.5 μm, 0.75 μm, 1 μm, 1.5 μm, 2 μm, 3 μm, 4 μm, or 5 μm to about 6 μm, 7 μm, 8 μm, 9 μm, 10 μm, 50 μm, or 100 μm, and is injected or otherwise introduced into the body (e.g., the brain), tissue, cell, plant, or culture medium containing the tissue or cell where it enhances ultrasound stimulation.

In various embodiments, the method involves delivering or applying acoustical energy (ultrasound) to a targeted tissue or cell. In general, the delivery or application of the acoustical energy to the targeted tissue or cell is non-invasive. In some embodiments the target tissue or cell forms part of a sensory unit. In various embodiments, the tissue or cell has been configured to express an acoustically sensitive transmembrane protein, e.g., an exogenous or heterologous acoustically sensitive transmembrane protein, such that when the tissue or cell is exposed to acoustical energy, a membrane potential of cell or cells comprising the tissue is modulated at least in part due to exposure of the acoustically sensitive protein to the acoustical energy. The tissue or cell may have been genetically or recombinantly modified to express the acoustically sensitive transmembrane protein. The acoustically sensitive transmembrane protein may be selected from DmFLYC1, DmFLYC2, DmOSCA, DcFLYC1.1, DcFLYC1.2, or an ortholog or homolog thereof.

The acoustical source may be selected from, as non-limiting examples, a piezoelectric transducer, e.g., a PZT-based transducer, a composite transducer, a micromachined ultrasound transducer, a capacitive micromachined ultrasonic transducer, and a micro-electro-mechanical system. The acoustical source may comprise a silicon-on-insulator type micro-electro-mechanical system. In some embodiments, the acoustical energy is delivered transcutaneously from an acoustical source. In some embodiments, the acoustical energy is delivered transcranially. In some embodiments, the tissue or cell is in the brain, e.g., the hypothalamus, of a subject. In some embodiments, the tissue or cell constitutes part of the central nervous system. In some embodiments, the tissue or cell constitutes part of the peripheral nervous system. In some embodiments, the acoustical source is disposed on or within a tissue or organ of the subject or plant. In some embodiments, the acoustical source is disposed on or within the brain of the subject. In a particular embodiment, a nerve cell expressing an acoustically sensitive, heterologous mechanosensory protein as described herein, e.g., a motor neuron, in one area, e.g., the spinal cord, is stimulated with acoustical energy (ultrasound), resulting in activation, inhibition, or silencing of nerve cells, e.g., motor neurons, in another area, e.g., in downstream muscle tissue. In some embodiments, the method involves using a plurality of acoustic emitters to activate cells or tissue(s) of a subject. In embodiments, the cells are neurons, e.g., motor neurons, in the spinal cord and/or in downstream muscles.

In an aspect, a method for sonogenetics-based neuromodulation in a patient is provided. In some embodiments, the method involves determination of a desired nervous system functional modulation which can be facilitated by sonogenetic excitation and/or inhibition. The method can further involve a selection of a neuroanatomic resource within the patient to provide such outcome. The method can involve causing cells of the neuroanatomic resource to render them sensitive to acoustical energy. The method can further involve delivering acoustical energy to the targeted neuroanatomy to cause controlled, specific excitation and/or inhibition of such neuroanatomy by virtue of the presence of the mechanoresponsive protein in cells thereof. In some embodiments, mechanosensory proteins modulate membrane potential of a neuron, or other type of cell, by transporting ions, e.g., potassium, sodium, calcium, chloride, bromide, fluoride, or iodide ions, through the membrane of the cell.

The practice of the aspects and embodiments as described herein employs, unless otherwise indicated, conventional techniques of molecular biology (including recombinant techniques), microbiology, cell biology, biochemistry and immunology, which are well within the purview of the skilled artisan. Such techniques are explained fully in the literature, such as, “Molecular Cloning: A Laboratory Manual”, second edition (Sambrook, 1989); “Oligonucleotide Synthesis” (Gait, 1984); “Animal Cell Culture” (Freshney, 1987); “Methods in Enzymology” “Handbook of Experimental Immunology” (Weir, 1996); “Gene Transfer Vectors for Mammalian Cells” (Miller and Calos, 1987); “Current Protocols in Molecular Biology” (Ausubel, 1987); “PCR: The Polymerase Chain Reaction”, (Mullis, 1994); “Current Protocols in Immunology” (Coligan, 1991). These techniques are applicable to the production of the polynucleotides and polypeptides as described herein, and, as such, may be considered in making and practicing the aspects and embodiments as described. Particularly useful techniques for particular embodiments will be discussed in the sections that follow.

The following examples are put forth so as to provide those of ordinary skill in the art with a complete disclosure and description of how to make and use the assay, screening, and therapeutic methods as described, and are not intended to be limiting.

### Compositions and Pharmaceutical Compositions

Compositions comprising cells expressing a heterologous or exogenous mechanosensory polypeptide as described herein are provided. In an embodiment, the composition is a pharmaceutical composition. Typically, the carrier or excipient for such a composition is a pharmaceutically acceptable carrier or excipient, such as sterile water, aqueous saline solution, aqueous buffered saline solutions, aqueous dextrose solutions, aqueous glycerol solutions, ethanol, or combinations thereof. The preparation of such solutions ensuring sterility, pH, isotonicity, and stability is affected according to protocols established in the art. Generally, a carrier or excipient is selected to minimize allergic and other undesirable effects, and to suit the particular route of administration, e.g., subcutaneous, intramuscular, intranasal, and the like.

A composition or pharmaceutical composition is administered at a dosage or effective amount that ameliorates, decreases, diminishes, abates, alleviates, or eliminations the effects of a disease, disorder, or condition, and/or the symptoms thereof. The composition may be provided in a dosage form that is suitable for parenteral (e.g., subcutaneous, intravenous, intramuscular, intrathecal, or intraperitoneal) administration route. The pharmaceutical compositions may be formulated and prepared according to conventional pharmaceutical practice (see, e.g., Remington: The Science and Practice of Pharmacy (20th ed.), ed. A. R. Gennaro, Lippincott Williams & Wilkins, 2000 and Encyclopedia of Pharmaceutical Technology, eds. J. Swarbrick and J. C. Boylan, 1988-1999, Marcel Dekker, New York). A pharmaceutical composition may be administered parenterally by injection, infusion or implantation (subcutaneous, intravenous, intramuscular, intraperitoneal, intrathecal, and the like). Compositions for parenteral use may be provided in unit dosage forms (e.g., in single-dose ampules), or in vials containing several doses and in which a suitable preservative may be added (see below). The composition may be in the form of a solution, a suspension, an emulsion, an infusion device, or a delivery device for implantation.

### EXAMPLES

### Example 1: Discovery of FLYCATCHER1 and FLYCATCHER2 (FLYC1 and FLYC2), and DmOSCA

The Venus flytrap has evolved rapid touch-induced movements. Bending of any one trigger hair of the Venus flytrap generates an action potential in sensory cells at the base of the trigger hair that propagates through the trap and is correlated with an increase in cytosolic calcium in leaf trap cells. Early recordings from Venus flytrap trigger hairs have suggested that, based on the time scale of action potential generation, mechanosensitive ion channels may play a role in transducing force into electrical signals. To identify possible ion channels required for the touch response in Venus flytrap, an effort was initiated to find genes that are preferentially expressed in trigger hairs.

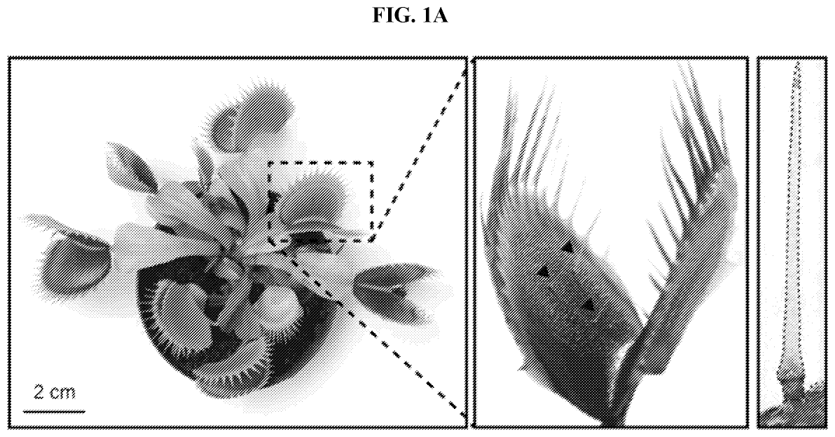

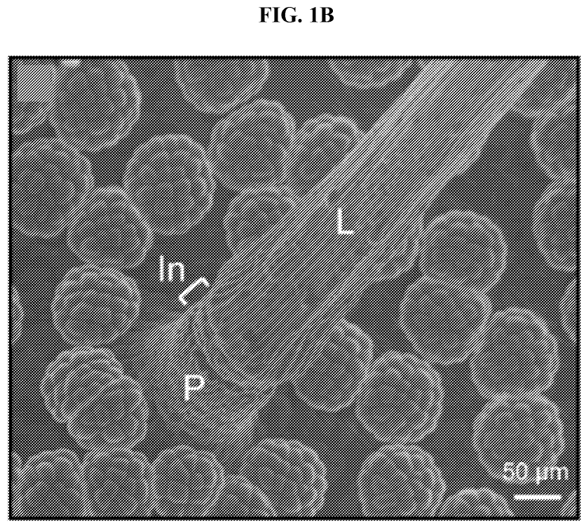

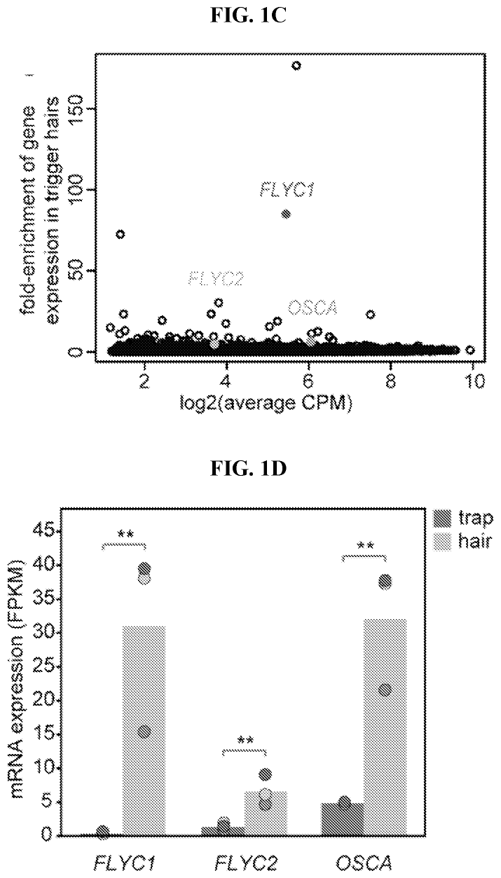

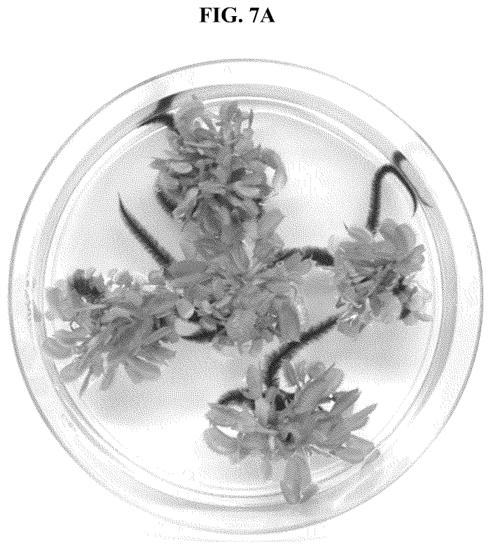

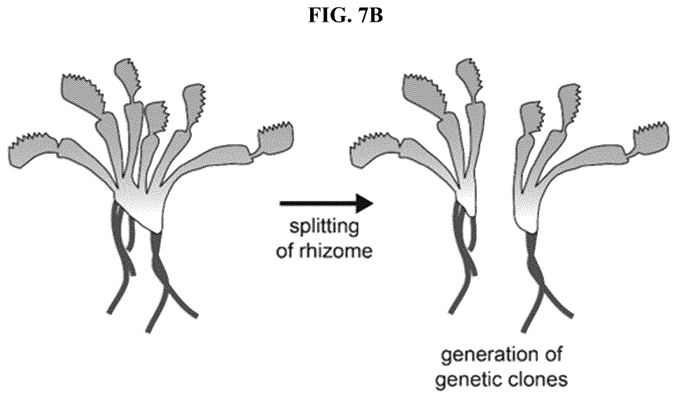

The Venus flytrap genome is very large—approximately 20 times the size of that of the commonly studied model plant Arabidopsis thaliana (Table 1)—and lacks available inbred strains. Therefore, a clonal propagation system was established for the collection of genetically identical material (FIGS. 1A, 1B, 7A and 7B). Using these clones, a de novo transcriptome was generated representing genes expressed in trap tissue from Illumina-based short sequencing reads. The transcriptome of the clonal strain consisted of almost 28,000 unique transcripts with open reading frames coding for predicted proteins of at least 100 amino acids. This is larger than the approximately 21,000 genes previously predicted by genome sequencing, reflecting the presence of multiple isoforms in the transcriptome, possible heterozygosity and strain differences, and/or differences in genome/transcriptome completeness, indicated by BUSCO analysis (see Materials and Methods). Comparing the transcriptome of the plant's trigger hairs with that of only the leaf traps revealed that 495 protein-coding genes were differentially-enriched by greater than 2-fold in the trigger hair, whereas 1,844 protein-coding genes were similarly enriched in the trap. Based on homology to Arabidopsis, many genes preferentially expressed in leaf traps were associated with photosynthetic function, while those more highly expressed in the trigger hairs included transcription factors and genes that may affect cellular and organ structure.

TABLE 1
Size estimates of two Arabidopsis (Col-0) samples compared to the
Venus flytrap strain (CP01).

| Species | DNA Content (pg/2C) | St. Dev. |
|---|---|---|
| A. thaliana (Col-0) | 0.38 | 0.010 |
| Sample #1 | | |
| A. thaliana (Col-0) | 0.40 | 0.007 |
| Sample #2 | | |
| D. muscipula (CP01) | 7.86 | 0.359 |

To find potential mechanosensitive ion channels, the trigger hair-enriched transcriptome was screened for transcripts coding for likely multi-pass transmembrane proteins. Of 45 such transcripts, three coded for possible candidates based on homology to Arabidopsis proteins. Two of these shared homology to MSL family proteins (transcript IDs comp20014_c0_seq1 and comp28902_c0_seq1), and one to the OSCA family (comp16046_c0_seq1). These genes were named FLYCATCHER1 and FLYCATCHER2 (FLYC1 and FLYC2), and DmOSCA, respectively. Enriched expression of homologs to the mechanically activated PIEZO channels were not observed. One of these MSL-related transcripts, FLYC1, was expressed 85-fold higher in trigger hairs than in trap tissue (FIGS. 1C and 1D), and was the second highest enriched gene (a putative terpene synthase was 176-fold enriched; see Materials and Methods). By contrast, the other two putative ion channels were less than 7-fold differentially expressed. To validate the transcriptome, the transcript sequence from cDNA of FLYC1 was verified using Sanger sequencing to determine the complete gene sequence. The absence of single nucleotide polymorphisms (SNPs) causing amino acid changes in exons in the strain was determined to be indicative of selection for the protein product (Materials and Methods).

### Example 2: Structural, Sequence, and Phylogenetic Analyses of FLYC1, FLYC2, and DmOSCA

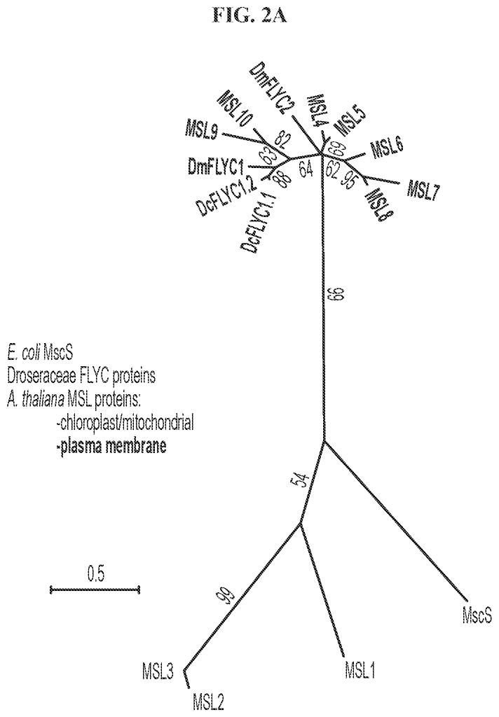

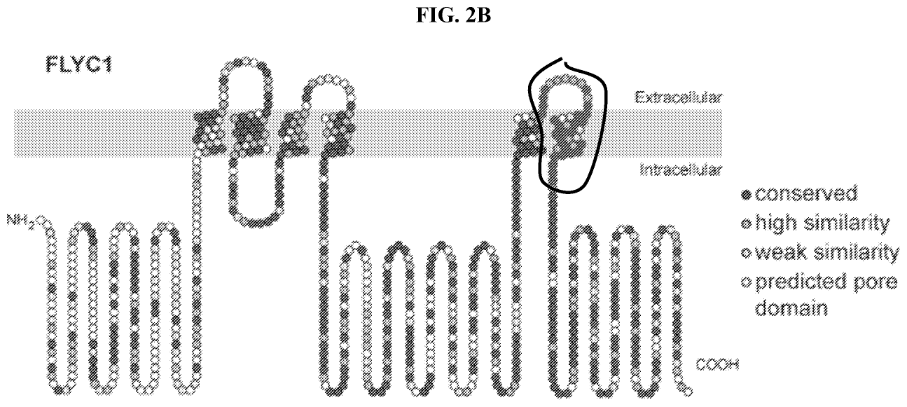

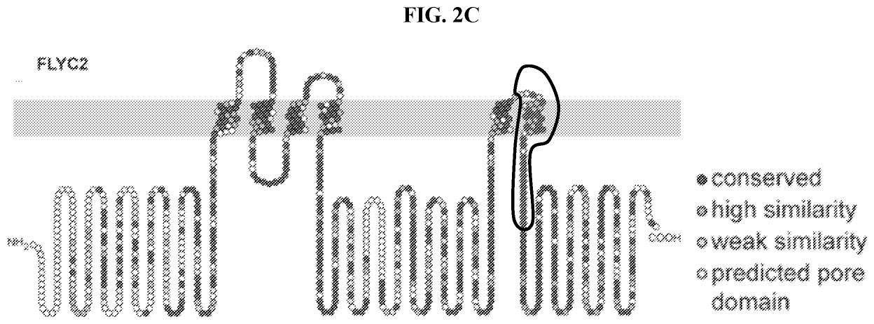

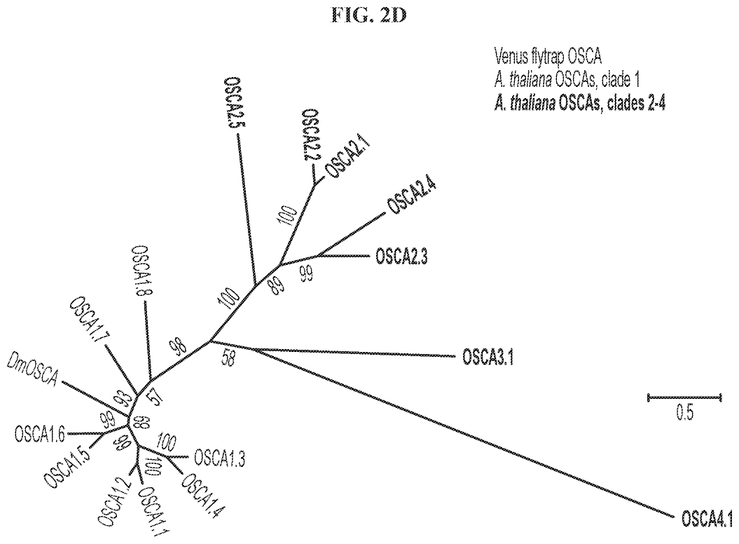

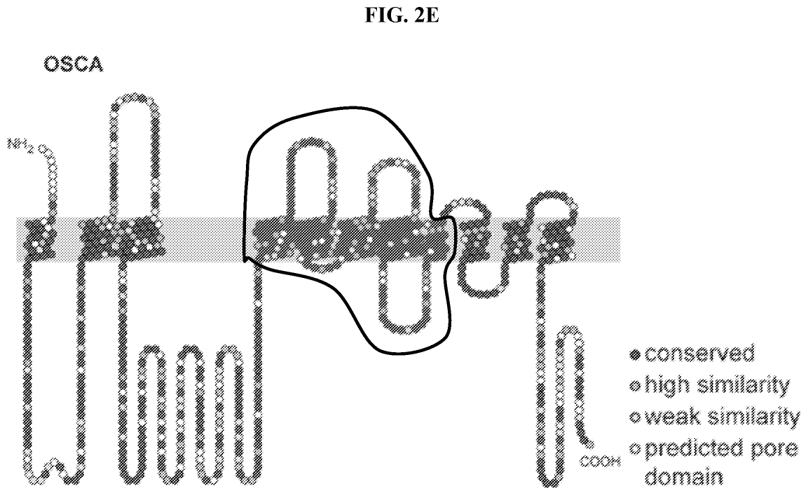

FLYC1 and FLYC2 coded for predicted proteins of 752 and 897 amino acids, respectively, with homology to Arabidopsis MSL10 and MSL5 (FIG. 2A). Ten MSL (MscS-like) proteins were discovered previously in Arabidopsis based on their similarity to the bacterial mechanosensitive channel of small conductance, MscS. Not wishing to be bound by theory, in Escherichia coli, MscS opens to allow ion release upon osmotic down-shock and cell swelling, thereby preventing cell rupture. In Arabidopsis, MSL8 functions in pollen rehydration, whereas the roles of the remaining MSLs in mechanosensory physiological processes is unclear. MSL10 forms a functional mechanosensitive ion channel with slight preference for chloride when heterologously expressed in Xenopus laevis oocytes. Furthermore, along with other members of the MSL family, MSL10 accounts for stretch-activated currents recorded from root protoplasts. While FLYC1 and FLYC2 were 38.8% identical, they shared 47.5% and 35.5% identity with MSL10 respectively, with most variation in the cytoplasmic N-terminus. Compared to MscS, FLYC1 and FLYC2 had three additional predicted transmembrane helices, six in total, with the C-terminus of the proteins sharing highest homology (FIGS. 2B and 2C).

DmOSCA encoded a predicted 754 amino acid protein with highest homology to Arabidopsis OSCA1.5 (64% identity), which belong to a 15 member family of OSCA proteins in Arabidopsis (FIGS. 2D and 2E). Although initial identification characterized the OSCA family as hyperosmolarity-activated calcium channels, it has been demonstrated that several members of the OSCA family are mechanosensitive ion channels that are non-selective for cations and that have some chloride permeability. Fly and mammalian orthologues, Tmem63, also encode mechanosensitive ion channels, suggesting that the molecular function of the OSCA genes is conserved. Furthermore, purification and reconstitution of AtOSCA1.2 in proteoliposomes induced stretch-activated currents, indicating that these proteins are inherently mechanosensitive. Notably, mutant OSCA1.1 plants have stunted leaf and root growth when exposed to hyperosmotic stress, possibly as a consequence of impaired mechanotransduction in response to changes in cell size. This suggests an ancestral role for these channels as osmosensors, similar to the MSL family.

### Example 3: Identification of Cells Expressing FLYC1, FLYC2, and DmOSCA

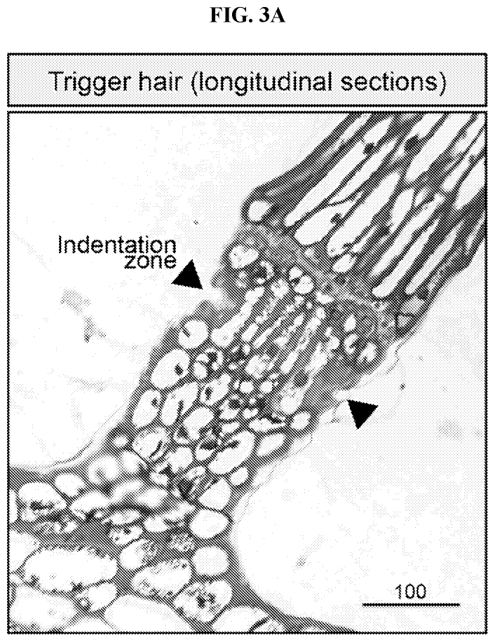

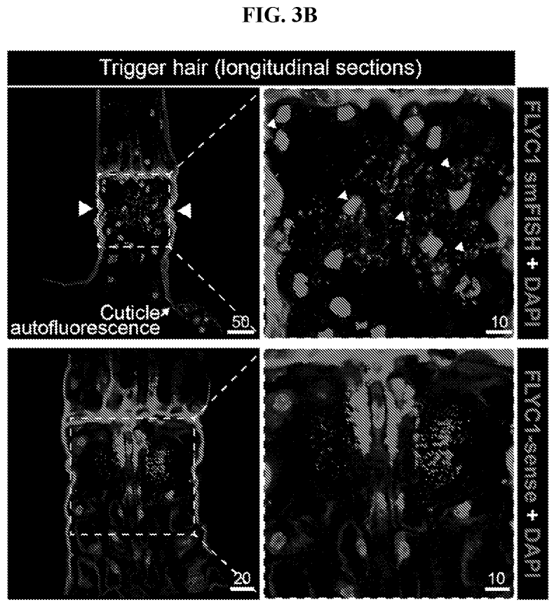

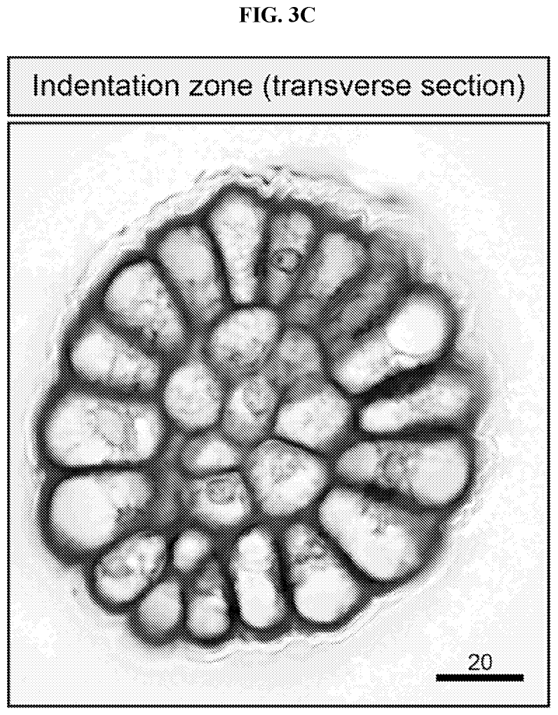

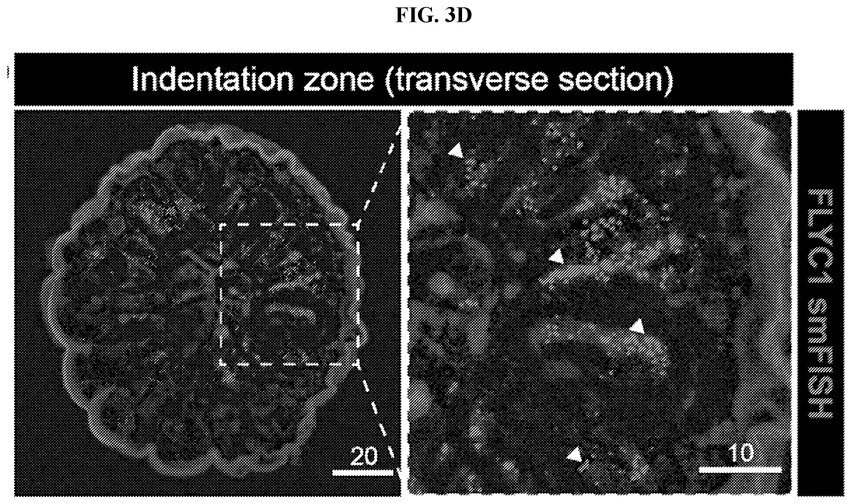

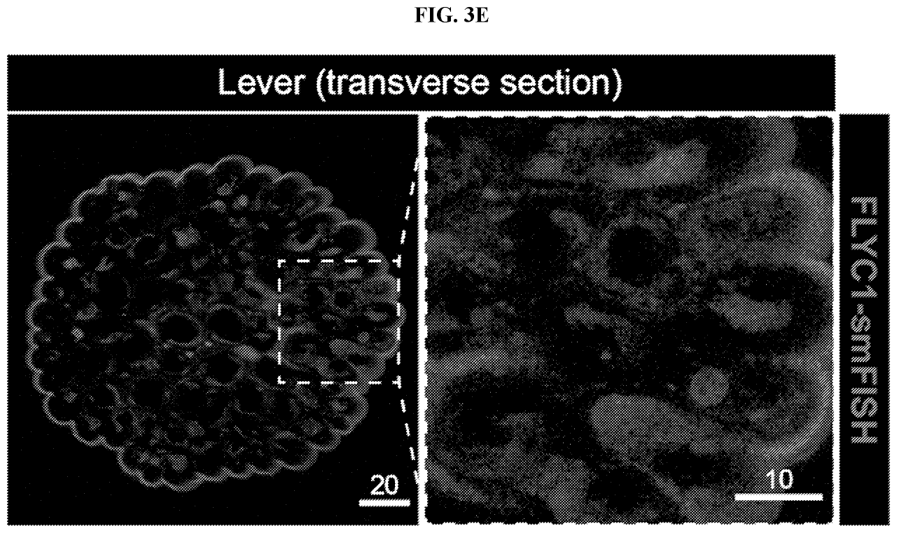

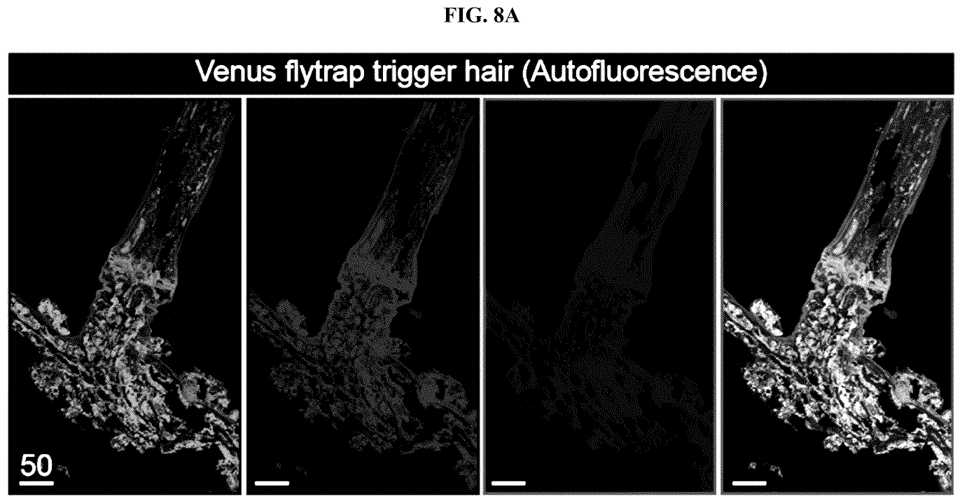

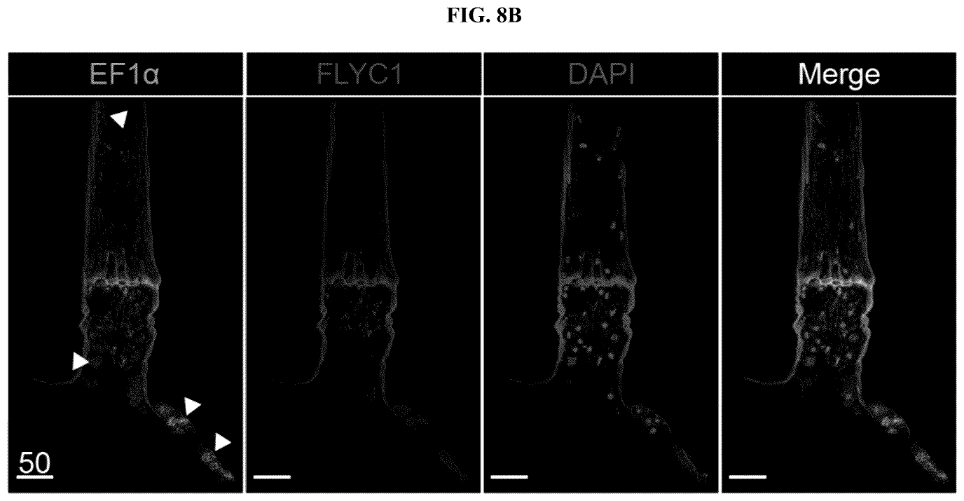

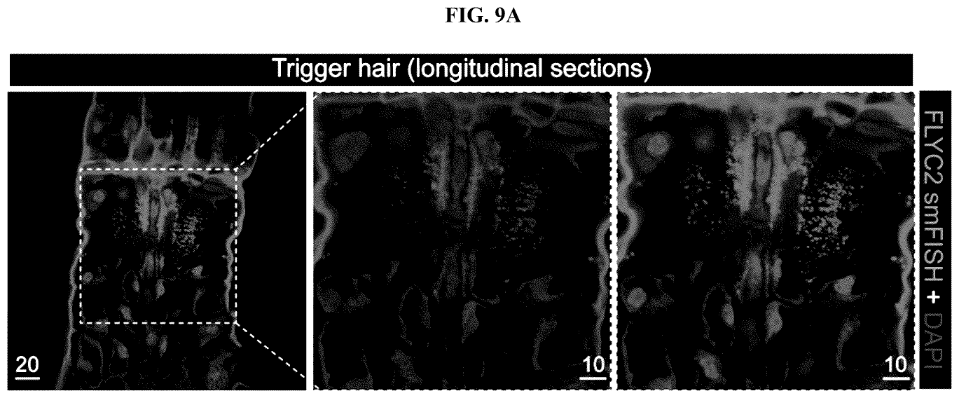

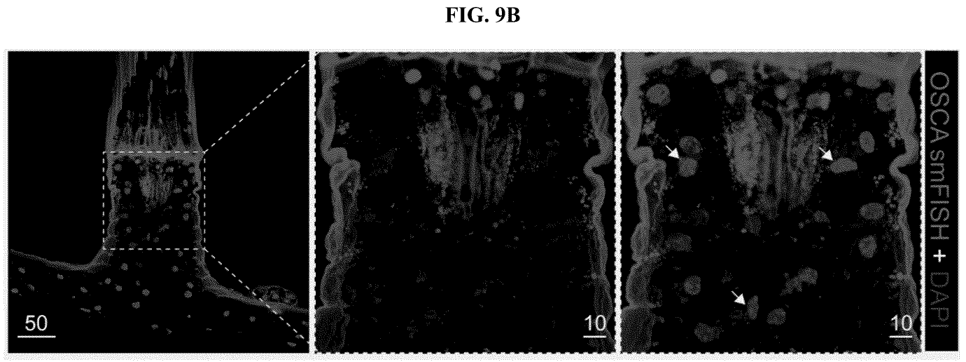

Experiments were conducted to identify the cells in the trigger hair that expressed the FLYC1, FLYC2, and DmOSCA genes. The trigger hair can be divided into two main regions: a cutinized lever and a podium on which the lever sits. An indentation zone at the top of the podium separates the two regions (FIGS. 3A and 3C). This zone is where most flexure of the trigger hair occurs. Electrophysiological recordings have demonstrated that mechanical stimulation of the trigger hair generates action potentials from a single layer of sensory cells at this indentation zone, and not from other cells of the podium or lever. By fluorescent in situ hybridization, FLYC1 transcript was detected specifically in indentation zone sensory cells (FIGS. 3B, 3C, and 3D). No transcript was observed in cells of the lever or lower podium (FIGS. 3B and 3E). In contrast, EF1α transcript, a housekeeping gene, was detected throughout the trigger hair and trap (FIGS. 8A and 8B). FLYC1-sense probes produced no signal above background (FIG. 3B). Using similar in situ hybridization methods FLYC2 transcripts were not detected, whereas DmOSCA was found at high levels within trigger hair sensory cells, but also low levels in other cell types (FIGS. 9A and 9B). These results are consistent with RNA-seq findings from whole trigger hairs, where FLYC2 and DmOSCA were less enriched over background trap tissue compared to FLYC1, and in the case of FLYC2, were only weakly expressed (FIG. 1D). Notwithstanding, the expression profile of FLYC1 and DmOSCA was consistent with these genes being at the site of touch-induced initiation of action potentials, resulting in trap closure.

### Example 4: Exogenous Expression of FLYC1, FLYC2, and DmOSCA in HEK-P1KO Cells

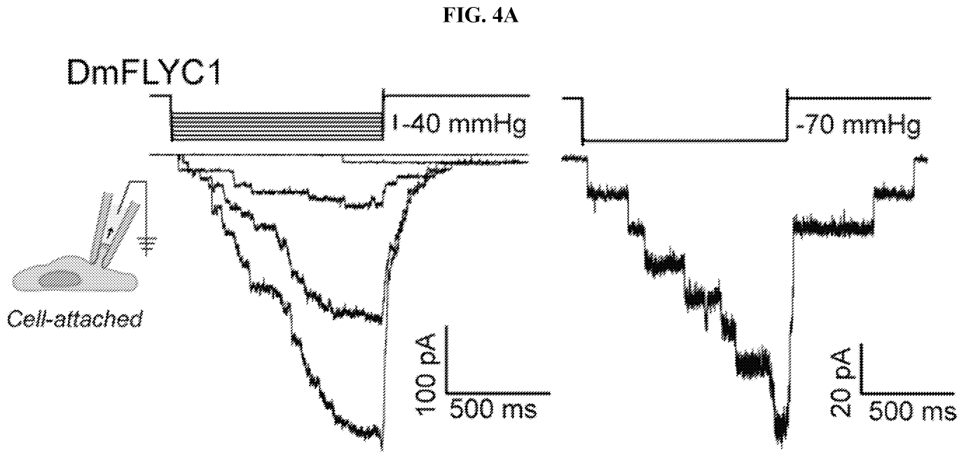

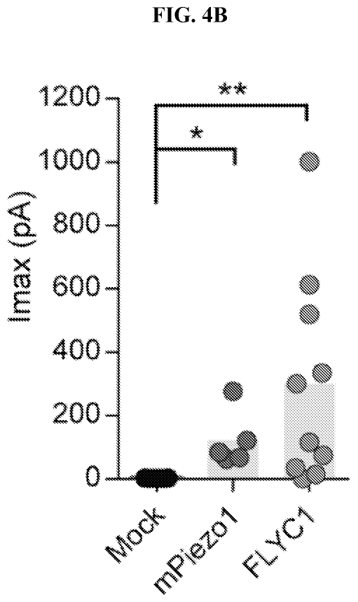

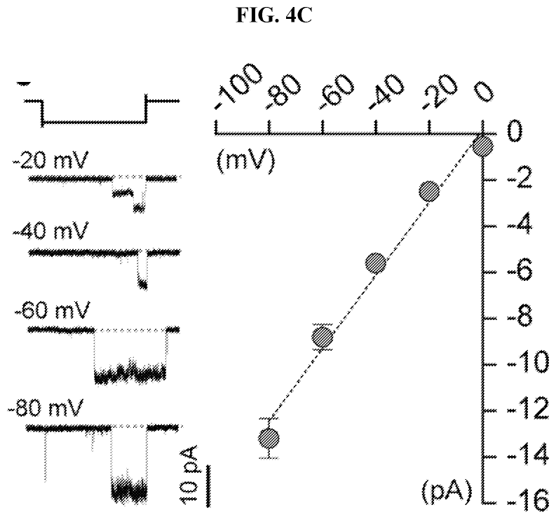

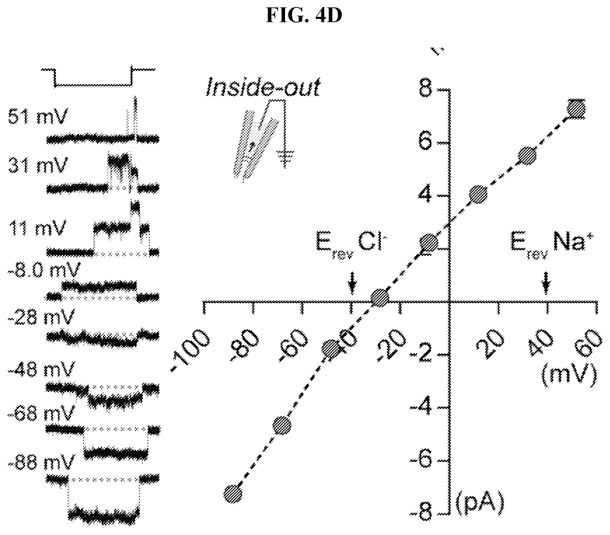

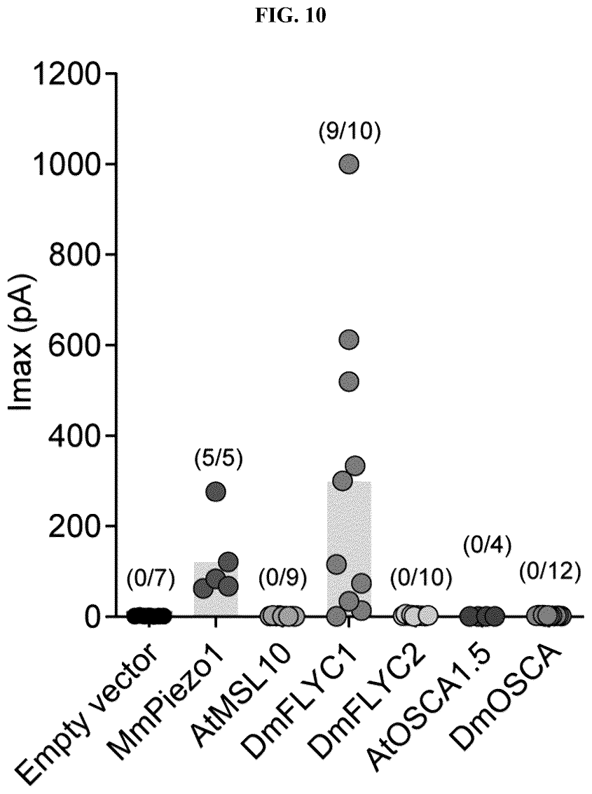

To test whether FLYC1, FLYC2, and DmOSCA were mechanically-activated ion channels and that conferred mechanosensitivity to naïve cells, human codon-optimized polynucleotide sequences of these genes were exogenously expressed in mechanically-insensitive HEK-P1KO cells. Robust stretch-activated currents were recorded from FLYC1-expressing cells when negative pressure was applied to the recording pipette in the cell-attached patch configuration (FIGS. 4A and 4B), but not from FLYC2-, or DmOSCA-expressing cells (FIG. 10). In these experiments, overexpression of human codon-optimized MSL10 and of OSCA1.5 subcloned from Arabidopsis thaliana did not produce stretch-activated currents in the system using HEK-P1KO cells (FIG. 10). Without intending to be bound by theory, expression of MSL10 in oocytes has been shown to elicit stretch-activated currents; therefore, trafficking of these plant proteins may be compromised following expression in mammalian cells. As would be appreciated by the skilled practitioner in the art, modifying polynucleotide expression techniques, the conditions used to express the plant proteins in HEK-P1KO, the use of other expression systems and host cells, and/or further codon-optimization, and the like, may lead to functional stretch-activated currents by certain of these exogenously-expressed plant-derived proteins. Given the high transcript enrichment, localization in sensory cells, and mechanosensitivity, FLYC1 was selected as a likely functional mechanosensor in Venus flytrap sensory hairs.

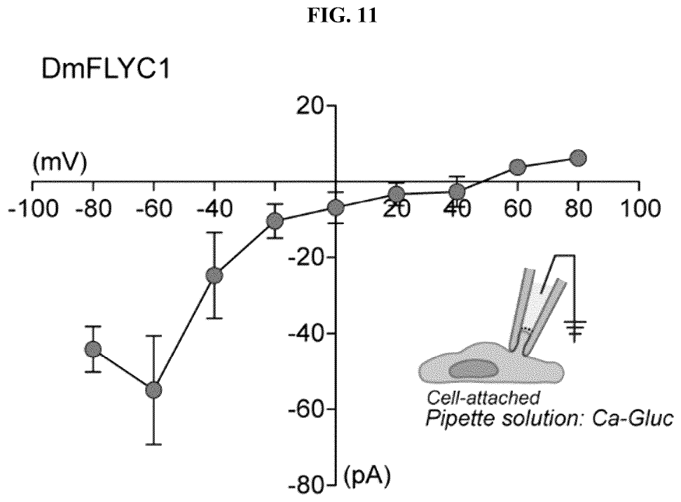

Further characterization of FLYC1 channel properties indicated that stretch induced FLYC1 activation occurred at −70±4 mmHg (N=9), and the channel had a conductance of 164±9 pS (N=6) (FIG. 4C). Similar to MSL10 stretch-activated currents in oocytes, FLYC1 currents had a slow rise time for activation and did not saturate, suggesting that these channels didn't inactivate. Upon removal of the stretch stimulus, the currents decayed with a time constant of 167±34 ms (N=5). Not wishing to be bound by theory, in land plants and green algae, chloride-permeable channel opening is associated with membrane depolarization, due to the efflux of chloride ions down their electrochemical gradient. Therefore, it was tested whether FLYC1 is permeable to chloride by recording stretch-activated FLYC1 currents from excised patches in asymmetrical NaCl solution. FLYC1 was demonstrated to exhibit a preference for chloride over sodium with a P_{Cl}/P_{Na} ratio of 9.8±1.8 (N=7) (FIG. 4D and FIG. 11), which was higher than ratios previously obtained for MSL10 (P_{Cl}/P_{Na}: 5.9) and MscS (P_{Cl}/P_{K}: 1.2-3.0).

### Example 5: Evaluation of the Pore-Lining Helix

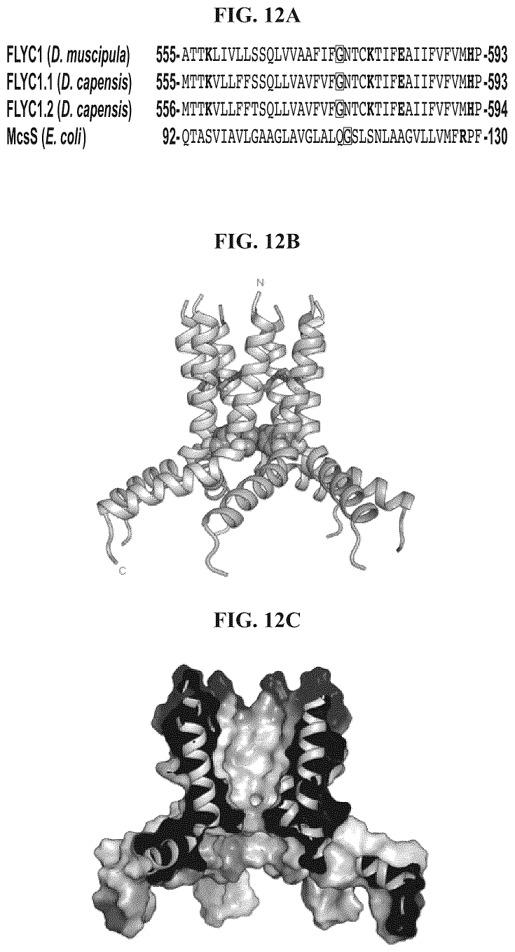

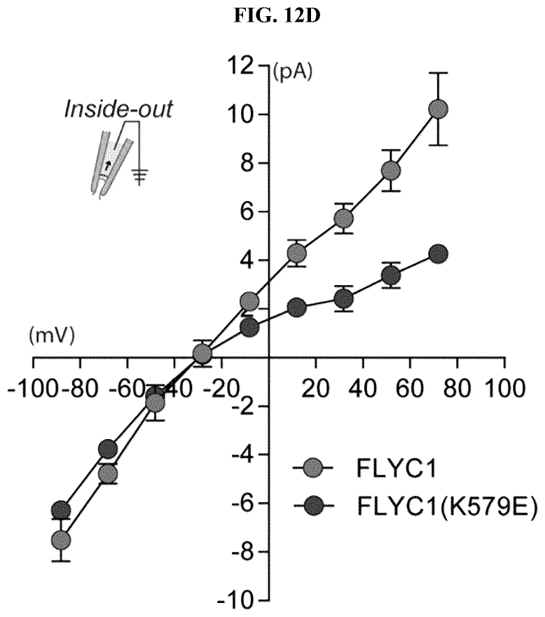

While bacterial MscS are known to be mechanically activated ion channels gated by membrane tension, much less is known about the structure and function of plant MSLs. Although homology modelling of MSL10 with MscS has been indicative of certain residues that alter channel conductance, molecular determinants of selectivity and gating in MSL10 remain largely unknown. Because of a high sequence similarity in the C-terminus of MscS, MSL10, and FLYC1 proteins (FIG. 12A), the extent to which the molecular architecture of the putative pore-lining helix was shared among these channels was evaluated. Homology-modelling of FLYC1 with the known MscS structure suggested that certain features of the pore-forming transmembrane domain (TM6) were conserved. These include hydrophobic residues within the part of the helix that lines the pore (TM6a), and a glycine kink at G575 followed by an amphipathic helix (TM6b) that lies parallel to the inner plasma membrane surface (FIGS. 12B and 12C). Based on the result that FLYC1 has a high preference for chloride, it was tested whether the positively-charged lysine residues on either side of the putative pore (K558 and K579) may confer pore properties. Lysine residues were substituted at positions 558 and 579 with glutamate residues and P_{Cl}/P_{Na} was measured. While selectivity for chloride in both mutants remained unchanged, the K579E mutant exhibited smaller single channel currents at positive membrane potentials (FIG. 12D), suggesting that K579 was in the vicinity of the pore. This analysis confirmed that the predicted TM6 of FLYC1 is part of the pore-lining region of the channel, consistent with mutagenesis results in MSL10. These findings were further supported by the cryo-EM (electron microscopy) structure of AtMSL1, which indicated a similar architecture of the last transmembrane (TM) of the channel.

### Example 6: Exogenous Expression of DcFLYC1.1 and DcFLYC1.2

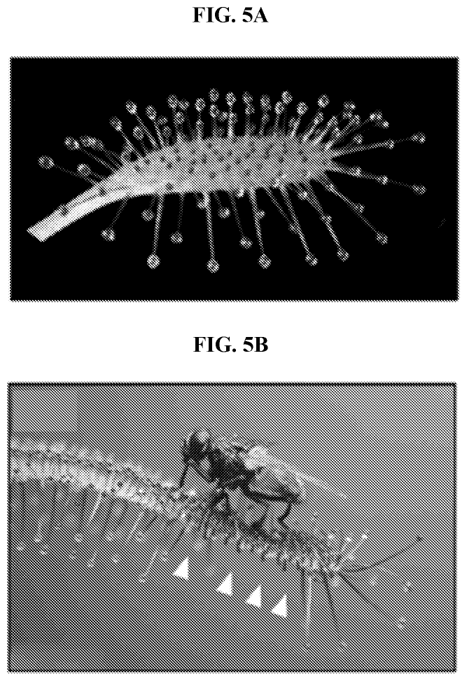

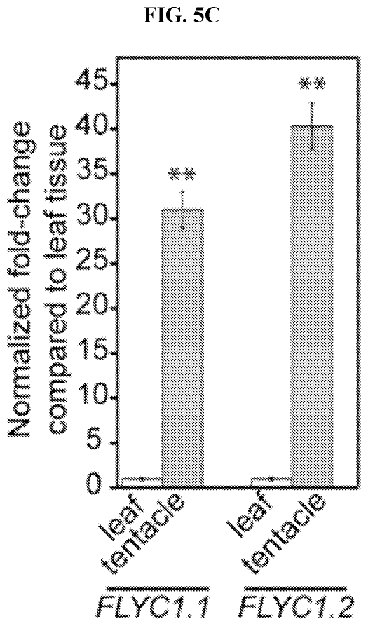

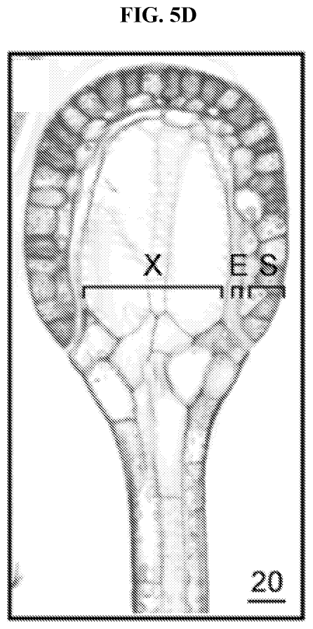

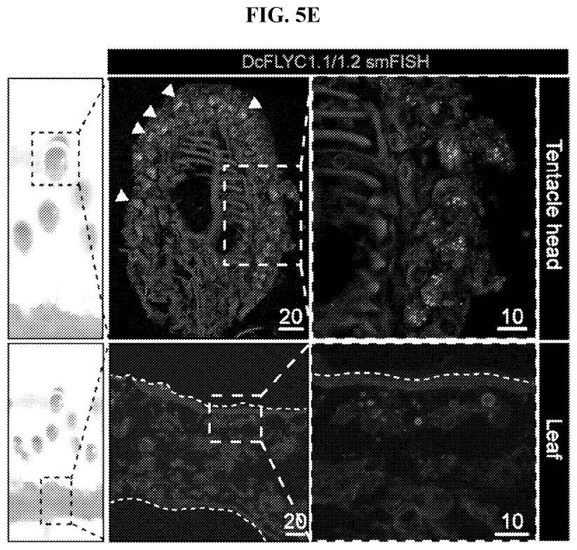

Because of the importance of FLYC1 for touch-induced prey recognition, its expression and function in mechanosensory structures was hypothesized to be conserved across carnivorous Droseraceae plants. To test this hypothesis, the largest genus in the family, Drosera, which includes approximately 200 species of sundew plants was investigated. Sundews are characterized by touch-sensitive projections on their leaf surface called tentacles (FIG. 5A). These tentacles typically secrete a glob of sticky mucilage from their head, which acts as a trapping adhesive when contacted by insect prey. Movement by the adhered insect results in action potentials along the tentacle, often accompanied by radial movement of the tentacle toward the leaf center. This traps the struggling insect against even more mucilage-secreting tentacles, allowing for digestion to occur (FIG. 5B).

The expression of two FLYC1 homologs in Cape sundew (Drosera capensis) was identified by quantitative reverse transcriptase-(qRT-) PCR. Consistent with findings from experimental studies of the Venus flytrap, these two transcripts had 30- to 40-fold higher expression in tentacles compared to tentacle-less leaf tissue (FIG. 5C). The cloned cDNAs of these two genes—which were named DcFLYC1.1 and DcFLYC1.2—coded for almost identical predicted protein products with 96.4% identity. They shared 66.2% and 65.6% identity to Venus flytrap FLYC1, respectively (FIG. 2A).

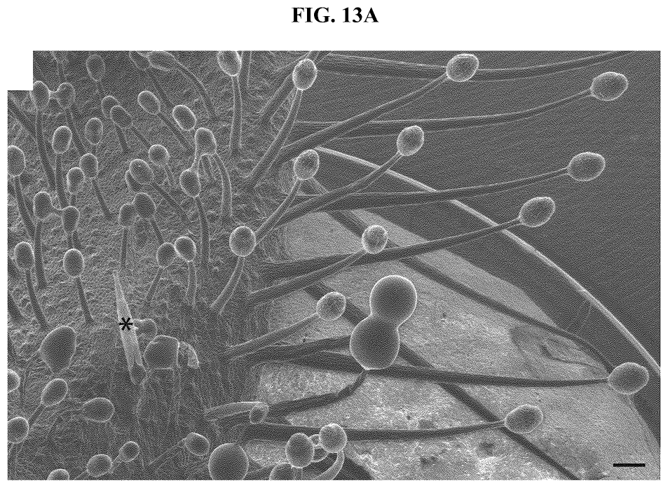

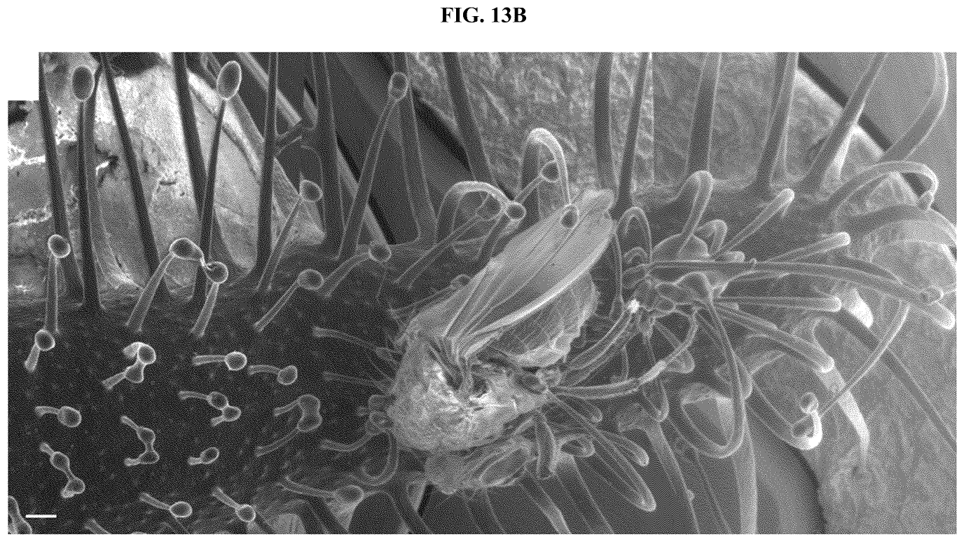

Cape sundew tentacles display variation in length depending on their position on the leaf (FIGS. 13A and 13B), but share a similar mechanosensory head structure, with two outer layers of secretory cells (FIG. 5D). The outermost secretory cells have previously been hypothesized to double as the site of touch sensation. Not only are these cells directly exposed to the mucilage environment on which the insect prey adheres and pulls, but they display a unique morphology of outer cell wall buttresses and plasma membrane crenellations (FIG. 14). In addition, cellulose fibrils extend from the outer cell wall into the cuticle in much the same way as they do in the Venus flytrap indentation zone. smFISH probes against DcFLYC1.1 and DcFLYC1.2 transcripts localized to the outer secretory cells, whereas no transcripts were observed in the leaf at the base of the tentacles (FIG. 5E).

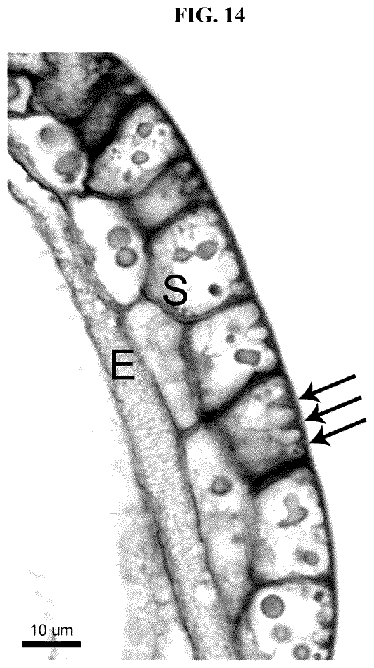

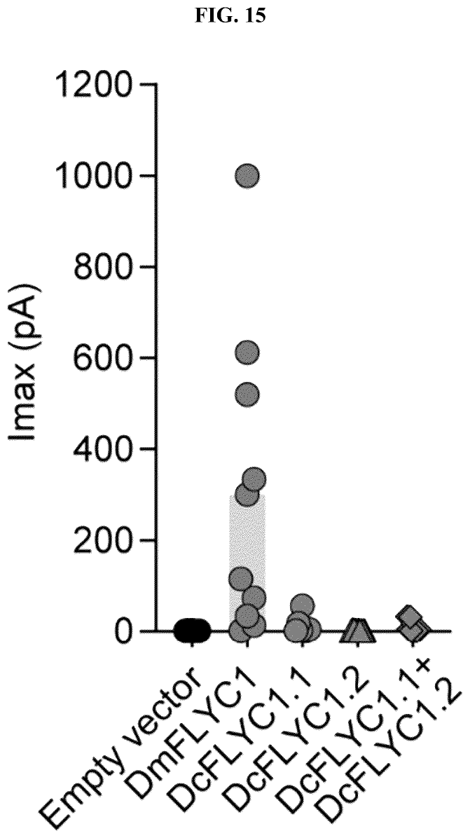

Heterologous expression of human codon-optimized DcFLYC1.1 and DcFLYC1.2 cDNA, independently or together in HEK-P1KO cells, did not result in stretch-activated currents (FIG. 15). Similar to the findings with DmFLYC2 and DmOSCA, the lack of activity may be due to incorrect folding and trafficking of these proteins. Technical difficulties in live-labeling these ion channels have limited the ability to present evidence for this (see Materials and Methods). Nonetheless, the expression data are consistent with a conserved role for FLYC1 in two divergent species in carnivorous Droseraceae plants.

Materials and Methods in Connection with Examples 1 to 6

### Plant Materials and Growth Conditions

Venus flytraps were propagated in tissue culture using methods modified from those previously described (Jang, G. W., Kim, K. S., & Park, R. D. (2003) “Micropropagation of Venus fly trap by shoot culture” Plant Cell Tissue and Organ Culture, 72(1), 95-98). Seeds (FlytrapStore.com, OR) were surface sterilized for 5 minutes (min) using 70% ethanol with 0.1% Triton X-100; rinsed; and then seeded onto ⅓× Murashige and Skoog (MS) salts and vitamins (Caisson Labs), 3% sucrose, and 4.3 g/L gellan gum. Following germination, plants were selected for robust growth in tissue culture as well as large size to facilitate tissue manipulations. A single strain originating from a single seed (CP01) was chosen for further propagation by continual splitting of rhizomes. After 9-12 months in culture, the largest rosettes were transferred to soil (fine-grade sphagnum peat moss) and grown under greenhouse conditions (25-30° C.; ˜16 h light 8 h dark, with overhead artificial red light added in evening hours to bring to 16 h light length). Soil was kept constantly moist using purified water. Plants were allowed to harden on soil for at least 2-3 months prior to experiments.

Drosera capensis var. rubra (Cape sundew) seeds (AK Carnivores, HI) were surface sterilized, germinated on plates, and seedlings transferred to soil and grown under greenhouse conditions.

### Imaging

For imaging of cell wall-stained tissue sections, freshly harvested plant tissue was dissected in 2% (v/v) paraformaldehyde and 1.25% (v/v) glutaraldehyde in 50 mmol/L PIPES buffer, pH 7.2 and fixed for 2 hours (h) in the same solution. Samples were dehydrated in a graded ethanol series and embedded in JB-4 Plus Embedding Media (Electron Microscopy Sciences) according to manufacturer's instructions, with the exception that infiltration was performed at room temperature over 7 days. Some dehydrated samples were then stained with 0.1% eosin in 100% ethanol prior to embedding to help visualize the material during sectioning. Sections were cut at 4-6 μm and dried from a drop of dH2O onto Probe On Plus slides (ThermoFisher Scientific). Tissues sections were stained with 0.01% aqueous Toluidine Blue O, cover-slipped and examined with an Olympus BX51 microscope.

Scanning electron microscopy (SEM) was performed by imaging fresh plant tissue in the variable-pressure mode of a field emission-scanning electron microscope (Sigma VP; Zeiss) at 5 Pa of nitrogen with the variable-pressure secondary electron detector.

For time-lapse movies and light images of D. capensis during feeding, plants were fed with Drosophila melanogaster (Canton-S) or house flies (Musca domestica) and imaged using an Olympus Tough TG-5 camera. To aid in feeding, all insects were momentarily paralyzed by placing them in a tube on ice for 5-10 min immediately prior to plant feeding.

### Estimation of Nuclear Genome Size

Nuclear genome size was estimated using flow cytometry methods similar to those reported previously (Arumuganathan, K., & Earle, E. D., 1991, Plant Molecular Biology Reporter, 9(3), 229-241. doi:10.1007/bf02672073), and were performed at Benaroya Research Institute, Seattle, WA. Nuclei from 50 mg of Arabidopsis leaf or Venus flytrap petiole tissue were suspended in 0.5 mL solution of 10 mM MgSO₄, 50 mM KCl, 5 mM Hepes pH 8.0, 3 mM dithiothreitol, 0.1 mg/mL propidium iodide (PI), 1.5 mg/mL DNAse free RNAse (Roche) and 0.25% Triton X-100. Nuclei were filtered through a 30 μm nylon mesh and incubated at 37° C. for 30 min. Stained nuclei were analyzed with a FACScalibur flow cytometer (Becton-Dickinson). As an internal standard, samples included nuclei from chicken red blood cells (2.5 pg/2C). For each measurement, the PI fluorescence area signals (FL2-A) from 1000 nuclei were collected and analyzed and the mean positions of the G0/G1 peaks for the sample and internal standard were determined using CellQuest software (Becton-Dickinson). Nuclear DNA size estimates are an average of 4 measurements.

Tissue Collection for RNA-Seq and qRT-PCR

To generate RNA-seq libraries from trigger hair tissue, trigger hairs were collected over the course of a month, 3-4 times a week during the hours of 6:30-9:30 μm under artificial red light. Only traps larger than 1.5 cm in length were used. 12 trigger hairs were dissected from the surface of two leaves at a time before snap-freezing in liquid N₂ (<5 min between dissection and freezing). Aliquots of trigger hairs were later pooled during RNA extraction. A sampling of traps (˜20) with the trigger hairs removed was collected simultaneously as comparison tissue. The experiment was performed in triplicate: 250 trigger hairs were collected for the first replicate, and 750 trigger hairs for each of replicates two and three.

For fed versus unfed trap samples, each tissue sample included two traps (>1.5 cm in length), each fed with a single house fly for 24 h. Fed traps were opened at the time of harvest and the fly carcass removed before snap-freezing the tissue. Samples were collected in duplicate.

Cape sundew tentacles were harvested by removing fresh leaves from plants and placing these directly into liquid N₂. While immersed in the liquid N₂, the leaves were agitated and/or scraped to break off the tentacles. The tentacles and remaining leaf material were then separated for RNA extraction. Each sample type was collected in quadruplicate.

RNA Extraction for qRT-PCR and RNA-Seq

Total RNA was extracted using a modified LiCl-based method similar to that described by others (Bemm, F. et al., 2016, Genome Res, 26(6), 812-825. doi:10.1101/gr.202200.115; Jensen, M. K. et al., 201), PLoS One, 10(4), e0123887. doi:10.1371/journal.pone.0123887). Briefly, frozen plant material was ground to a powder and 700 μL of RNA extraction buffer added (2% CTAB, 2% polyvinylpyrrolidone K25, 100 mM Tris HCl pH 8.0, 25 mM Na-EDTA pH 8.0, 2M NaCl, 2% v/v β-mercaptoethanol). If necessary, tissue aliquots were pooled at this stage, or later during ethanol precipitation. Samples were vortexed 2 min and then incubated 65° C. for 10 min, vortexing occasionally. Debris was removed by centrifugation and 600 μL chloroform was added to the supernatant and mixed. The sample was centrifuged at 10,000 rpm for 10 min. ⅓ vol 7.5 M LiCl was added to the aqueous phase which was then incubated overnight at 4° C. with gentle mixing. RNA was pelleted by centrifugation at 10,000 rpm for 10 min at 4° C. The RNA pellet was washed with 70% ethanol, air-dried, and re-suspended in 100 μL H2O. The RNA was then re-precipitated by adding 0.1 vol 3M NaAcetate (pH 5.2), 2.5 vol 100% ethanol and an optional 1.5 μL GlycoBlue (Thermo Fisher) for low yield samples; mixing; and incubating for 1 h at −80° C. The RNA was pelleted by centrifugation at 12,000 rpm for 20 min at 4° C. The pellet was washed with 70% ethanol, air dried, and re-suspended in 30-50 μL H₂O. These samples were then used directly to generate cDNA for qRT-PCR. Alternatively, samples for RNA-seq were re-suspended in 22 μL H2O, and to this was added 2.5 μL 10× TURBO buffer and 1 μL TURBO DNase (Thermo Fisher). Samples were incubated at 37° C. for 20-30 min. 2.5 μL Inactivation Reagent was then added, and samples incubated for a further 5 min at room temperature, mixing occasionally. The resin was removed by centrifugation at 10,000 rpm for 1.5 min and transferring the supernatant to a new tube. RNA quality and yield was assessed by Agilent TapeStation before proceeding with sequencing library generation.

### RNA-Seq and Venus Flytrap Trap Transcriptome Assembly

Stranded mRNA-Seq libraries were prepared using Illumina TruSeq Stranded mRNA Library Prep Kit according to the manufacturer's instructions. Libraries were quantified, pooled and sequenced at paired-end 125 bp reads using the Illumina HiSeq 2500 platform at the Salk NGS Core. Raw sequencing data was demultiplexed and converted into FASTQ files using CASAVA (v1.8.2). All samples were sequenced simultaneously on a single flow cell lane. The average sequencing depth was 8.8 million reads per library. Library quality was assessed using FastQC (v0.11.5) and Illumina adapter sequences and poor quality reads trimmed using Trimmomatic (0.36.0) with the suggested parameters by Trinity, an application for generating de novo transcriptomes widely used in the art.

Most aspects of the de novo transcriptome assembly and RNA-Seq analysis were performed using CyVerse cybercomputing infrastructure (S. Goff et al., 2011, Frontiers in Plant Science, 2, doi: 10.3389/fpls.2011.00034). To acquire the most complete representation of the Venus flytrap trap transcriptome, reads collected from house fly-fed and unfed traps were included, in addition to the paired trigger hair and trigger hair-less trap samples to build the trap transcriptome (Table 2). Trinity (v2.5.1) was used for the de novo build, using default settings and an assembled contig length of greater than 300 nt. The final transcriptome included 80,592 contigs, which were grouped into 77,539 components. Herein, each Trinity component is generally referred to as a “gene”. The maximum contig length was 15,873 nt; average and median lengths 867 and 536 nt, respectively; and N50 1,197 nt.

TABLE 2
Summary of sequencing reads used to build the de novo transcriptome
(NCBI Transcriptome Shotgun Assembly Sequence Database accession #
GHJF00000000, corresponding to submission number SUB5415411).

| | Paired | | | SRA accession |
|---|---|---|---|---|
| Sample | replicates | Tissue | # paired reads | # |
| unfed1 | no | 2 traps | 9,079,584 | SRR8834216 |
| unfed2 | no | 2 traps | 9,424,911 | SRR8834215 |
| fed1 | no | 2 traps | 9,597,638 | SRR8834218 |
| fed2 | no | 2 traps | 9,887,029 | SRR8834217 |
| trap1 | yes (A) | ~20 trapsª | 4,410,249 | SRR8834220 |
| trigger_hair1 | yes (A) | 250 hairs | 3,702,412 | SRR8834221 |
| trap2 | yes (B) | ~20 trapsª | 10,974,314 | SRR8834219 |
| trigger_hair2 | yes (B) | 750 hairs | 9,711,759 | SRR8834214 |
| trap3 | yes (C) | ~20 trapsª | 11,418,472 | SRR8834222 |
| trigger_hair3 | yes (C) | 750 hairs | 9,975,164 | SRR8834213 |

^{a}Trigger hairs removed.

CEGMA (Core Eukaryotic Genes Mapping Approach) and BUSCO (Benchmarking Universal Single-Copy Orthologs) analyses were used to assess the completeness of the Venus flytrap trap transcriptome. Using CEGMA, of 248 ultra-conserved core eukaryotic genes tested for, all were present in the transcriptome, including 246 of which the coding sequences were defined as complete by CEGMA criteria. In a search for the presence of near-universal single copy orthologs using BUSCO (v3.0; lineage: plantae; species: Arabidopsis), 94.7% of BUSCOs were present, 92.6% of which were defined as complete. This number is greater than that reported for the previously published Venus fly trap genome sequence (Palfalvi, G. et al., 2020, Curr Biol, 30(12), 2312-2320 e2315, doi:10.1016/j.cub.2020.04.051).

To further test the quality of the transcriptome, reads were aligned for each sample back to the transcriptome using Bowtie 2 (v2.2.4). For each sample, 80-90% of the paired reads mapped back concordantly, while the overall alignment rate was >90%. These values were similar when the reads for each sample from a second sequencing run were independently aligned.

TransDecoder (v1.0) was used to find open reading frames (ORFs) that coded for possible proteins or incomplete protein fragments of at least 100 amino acids in length on the +strand. In total, 33,710 ORFs (open reading frames) fulfilling these criteria were found (this number increased only a small amount, to 34,080, if the-strand was also included). Of these, 14,886 were identified as complete. These values are similar to those previously reported for a Venus flytrap transcriptome generated from multiple tissues (Jensen, M. K. et al., (2015), Transcriptome and genome size analysis of the Venus flytrap, PLoS One, 10(4), e0123887. doi:10.1371/journal.pone.0123887).

To assign homologous sequences from Arabidopsis to protein-coding transcripts, complete and partial polypeptides were blasted against the Arabidopsis TAIR10 proteome using default settings in Blastp (v2.2.29), with an e-value threshold of <0.01. The top hit only was retained for downstream analysis. To predict the number of transmembrane passes per protein, TMHMM v. 2.0 was used.

The de novo Venus flytrap trap transcriptome is available through the National Center for Biotechnology Information (NCBI) Transcriptome Shotgun Assembly (TSA) database with accession number GHJF00000000. The uploaded transcriptome has the following modifications from that described above and used for differential gene expression analysis below: the last 42 nt of contig comp11005_c0_seq1 and the first 21 nt of comp46326_c0_seq1 were removed (possible adapter sequences), and 18 contigs were flagged as possible contaminants and deleted. The uploaded transcriptome includes 80,574 contigs.

### RNA-Seq Differential Gene Expression Analysis

Methods supported by Trinity were used for finding genes differentially expressed between trigger hair and trigger hair-less trap tissue samples. To increase the sequencing read count number over any given contig, a second sequencing run of all samples was performed (SRA accession numbers SRR8834210, SRR8834209, SRR8834208, SRR8834207, SRR8834212 and SRR8834211). The concatenated reads were trimmed from both sequencing runs using Trimmomatic as described above, and aligned these to de novo Venus flytrap trap transcriptome using Bowtie. RSEM (v1.2.12) was used to find expected gene counts, and edgeR to find differentially-expressed genes using a paired experimental design for statistical testing. For the analysis in edgeR (v3.12.1), the gene list was first filtered to include only those Trinity components whose transcript(s) included an ORF (open reading frame) coding for a protein (or fragment thereof) of at least 100 amino acids in length. For components with more than one such protein assigned to them, the longest ORF (open reading frame) was assumed to be the most relevant, and was used as the basis for assigning an Arabidopsis homolog to the gene/component. Genes that had counts per million (CPM)<2 in over half the samples were excluded from the analysis. A table of genes passing these criteria showing gene expression values as mean CPM in traps and trigger hairs, as well as differential expression in trigger hairs versus traps calculated using edgeR algorithms with false discovery rate (FDR) can be found at the NCBI Gene Expression Omnibus (GEO) database with GEO Series accession number GSE131340. This repository also includes a list of all protein-coding genes and blast results against the Arabidopsis proteome. Genes that had a fold-enrichment difference of 2-fold or more, in addition to FDR <0.05, were designated as having trigger hair- or trap-enriched expression. Fold-enrichment was calculated using edgeR algorithms, and not directly from mean CPM values. For gene ontology (GO) analysis, GO terms were assigned to each gene based on its homologous Arabidopsis protein (Arabidopsis TAIR10 annotation data downloaded April 2018). GO enrichment was performed using BiNGO 3.03 (biological process terms only) with Benjamini and Hochberg corrected p value <0.05.

The highest differentially-expressed gene in the trigger hair transcriptome coded for a protein with homology to a terpene synthase (transcript ID comp18811_c0_seq1; approximately 175-fold enrichment). It is possible that the expression of this gene is a vestigial remnant of the proposed evolutionary history of the trigger hair from an ancestral, tentacle-like secretory structure. However, enriched expression of other genes obviously associated with volatile production were not observed. When the transcript was blasted against the Arabidopsis TAIR10 genome, a reported hit against Arabidopsis MSL10 was found, due to a conserved 17 nt stretch. Thus, a shared regulatory sequence may exist between this transcript and MSL-related genes.

Cloning of Venus Flytrap cDNAs, and Sequencing of DmFLYC1 Genomic DNA

cDNA from Venus flytrap whole-trap tissue was prepared from RNA using the Maxima First Strand cDNA Synthesis Kit (Thermo Fisher). FLYC1 cDNA (Trinity ID #comp20014_c0_seq1) was then PCR amplified using primers CP1010 and CP1011 (Table 3), which anneal in the predicted 5′ and 3′ UTRs, and was ligated into pCR-Blunt II-TOPO (Invitrogen). A clone was generated that matched the predicted sequence from the de novo transcriptome, as assessed by Sanger sequencing methods. Similarly, EF1α (comp10702_c0_seq1) was PCR amplified from cDNA template using primers CP1144 and CP1145, and ligated into pCR-Blunt II-TOPO. This EF1α transcript was chosen as a ubiquitously expressed control gene for the RNAscope experiments due to its high expression in all RNA-seq tissue samples. The EF1α cDNA insert was sequenced using flanking M13F and M13R primers that anneal in the pCR-Blunt II-TOPO vector, and verified that the first ˜1.1 kb and last ˜350 bp of the cDNA matched the prediction from the de novo transcriptome.

TABLE 3
Primer sequences used to amplify FLYC1 cDNA

| Primer | SEQ ID NO: | Sequence (5′→3′) |
|---|---|---|
| CP0994 | 15 | CCAGTGTCACCTTATAGGGAAGAAGCG |
| CP0995 | 16 | CCCTCGACGTAGTTCCCCTAGC |
| CP1009 | 17 | cacatcgatTCACATATTGCGGATACTAATTTCTTGGGGC |
| CP1010 | 18 | acacccgggGCTAGCTTTCATCCACCGAATAAACACC |
| CP1011 | 19 | cacatcgatACATCATTGACCAGAAGCAAGGCACTC |
| CP1033 | 20 | TGGCATCTTCATTCCATTTGAATAGGTTCTTTG |
| CP1034 | 21 | GCATACCCGACATGGTCGACAAGC |
| CP1035 | 22 | TGACAAGTTTGTTTAACTGCTTCACTGCTG |
| CP1144 | 23 | AGGTCTTTAGATTAACTCTTCAACATGGGTAAGG |
| CP1145 | 24 | ACAAGACTTCATTTTGCACCCTTCTTTATCG |
| CP1172 | 25 | GATTGAGCAAACAAAAGGCGCATGAAG |
| CP1173 | 26 | AAATTTTGACCTACGTTGACCGTCAGC |
| CP1174 | 27 | TGATCAGGCTGCGCTTAAACATGC |
| CP1176 | 28 | ACATTCTACTTTGTTTGCAATTGTTTTCCCACTC |
| CP1177 | 29 | AAGAAACATTAAGCTGCACCTGCTCC |
| CP1208 | 30 | GAGAGGTCCACCAACCTTGACTGG |
| CP1209 | 31 | AGCAACGGTCTGACGCATGTCC |
| CP1218 | 32 | GTGCCAGTGGGAAGAGTTGAGAC |
| CP1219 | 33 | CAGAGAAAGTCTCGACAACCATGGG |
| CP1224 | 34 | CAAATCATTGAAAACGTGAAGGGAAGCACTG |
| CP1225 | 35 | CCTATGAGATACTTAGCCTGTTAGCCATGC |
| CP1233 | 36 | GCCCTTCTATAGTAGTCTCACCTCTTCG |
| CP1237 | 37 | GCCATGCGCAGCATATGTACTAGC |
| CP1240 | 38 | GCTATTTCTTATTCTCCTGAGCACAACATACTG |
| CP1242 | 39 | TGATCGCTGTGTCGTAGATGGAACAATG |
| CP1243 | 40 | CTAATGGATTGCAAACTAGGAGATGCTTAGC |

Venus flytrap genomic DNA was extracted from fresh plant tissue using a CTAB-based method (Murray, M. G., & Thompson, W. F. (1980), Rapid isolation of high molecular weight plant DNA, Nucleic Acids Res, 8(19), 4321-4325). Using this as template, overlapping fragments covering the gene were PCR-amplified and sequenced using Sanger sequencing. Overlapping and nested primer sets were: CP1010 and CP0995; CP0994 and CP1034; CP1033 and CP1035; CP1172 and CP1173; CP1176 and CP1177; and CP1174 and CP1009 (see Table 3). The presence of two overlapping peaks on a chromatogram was used as evidence of heterozygosity and allelic variation. The most 3′ primer (CP1009) annealed over the stop codon and last 31 bases of the coding sequence, and, as such, the existence of additional SNPs within this stretch cannot be ruled out. 32 SNPs in total were detected in the FLYC1 gene, of which only 2 were found in the coding region and were silent (i.e. coded for the same amino acid).

For electrophysiological characterization, FLYC1, FLYC2, and OSCA cDNAs were gene synthesized (human codon optimized) into pIRES2-mCherry vector from Genewiz. K558E and K579E substitutions in FLYC1 were generated using Q5 Site-Directed Mutagenesis Kit (New England BioLabs) according to the manufacturer's instruction and confirmed by full-length DNA Sanger sequencing.

Identification of Drosera capensis cDNA Sequences and Design of qRT-PCR Primers

To find Drosera FLYC1 homologs, the first exon of Venus flytrap FLYC1 was blasted against the scaffold assemblies of the D. capensis genome (Butts, C. T. et al., 2016, Proteins, 84(10), 1517-1533, doi:10.1002/prot.25095). Because the first exon codes for the N-terminal cytoplasmic domain, which is most divergent among MSL family members, it may therefore best differentiate among different MSL genes and identify the best FLYC1 homolog. Four close homologous sequences were found using this method (e-value of 0.0; >60% query cover; NCBI blastn), two of which were located on scaffold LIEC01006169.1, one on LIEC01010092.1, and another on LIEC01012078.1. Position 42843-46002 of scaffold LIEC01006169.1 (reverse strand; start to stop codon) and position 23270-26408 of scaffold LIEC01012078.1 (forward strand; start to stop codon) were predicted to code for transcripts closely resembling the complete Venus flytrap FLYC1 sequence based on inferred exon structure and conserved sequences. The genes were called DcFLYC1.1 and DcFLYC1.2, respectively.

To determine the exact DcFLYC1.1 and DcFLYC1.2 sequences coded for in the D. capensis plants, the cDNAs were amplified using primers predicted to bind in the 5′ and 3′ UTRS (primers CP1240 and CP1243; and CP1224 and CP1225, respectively). Each cDNA was amplified from template derived from two different plants, and these PCR products were independently cloned into pCR-Blunt II-TOPO vector. For DcFLYC1.1, 1 of 2 and 2 of 2 clones from each of the two reactions from independent templates shared the same cDNA sequence as determined by Sanger sequencing. For DcFLYC1.2, 3 of 7 and 4 of 8 clones from each of the two reactions shared the same cDNA sequence. Other clones had additional base differences not seen in any other clone. While these might represent endogenous variation in this tetraploid species, they are more likely a result of PCR amplification errors or low-fidelity transcription and reverse transcription processes.

DcFLYC1.1 and DcFLYC1.2 cDNAs share high sequence similarity. To resolve between the two by qRT-PCR, primer pairs were designed where one of the two primers annealed in a less-conserved stretch of residues in the 5′ or 3′ UTR (CP1242 and CP1237, and CP1224 and CP1233, respectively). The resolution of the two cDNAs using these primers was confirmed by directly sequencing the two PCR products by Sanger sequencing. DcFLYC1.1 and DcFLYC1.2 cDNAs were gene synthesized by Genewiz (human codon optimized) and subcloned into pIRES2-mCherry vector for electrophysiological characterization.

To design qRT-PCR primers for the D. capensis EF1α reference transcripts, a PCR product from cDNA template was amplified using primers CP1208 and CP1209. Sanger sequencing of this product suggested the presence of at least two highly similar EF1α transcripts, as evidenced by double peaks on the chromatogram. These may represent the products of different genes or alleles. To avoid biases against any one EF1α transcript, the qRT-PCR primers were refined to anneal over unambiguous bases (CP1218 and CP1219).

qRT-PCR

Analysis of Cape sundew leaves and tentacles by qRT-PCR was performed using SYBR green-based protocols. Briefly, 500-1000 ng of RNA was used to generate cDNA using the Maxima First Strand cDNA Synthesis Kit (Thermo Fisher), from which dilutions were used as template for qPCR. Comparisons were performed in biological quadruplicate. Cycling reaction temperatures were 95° C. for 10 sec, 60° C. for 20 sec, and 72° C. for 30 sec. Fold-changes in gene expression were calculated using the ΔΔCt method.

### Bioinformatics Analyses: Homology Modelling, Protein/Nucleotide Comparisons, Protein Topology Predictions, and Evolutionary History

Unless otherwise specified, all nucleotide and amino acid comparisons were made using the default settings of MUSCLE. For phylogenetic tree analysis of MscS domain and OSCA proteins shown in FIGS. 2A and 2D, Maximum Likelihood trees were built from MUSCLE alignments of MscS domains (Haswell, E. S., & Meyerowitz, E. M. (2006). MscS-like proteins control plastid size and shape in Arabidopsis thaliana. Curr Biol, 16(1), 1-11. doi:10.1016/j.cub.2005.11.044) and full-length OSCA proteins using MEGA X software (LG+G model, 4 discrete Gamma categories) and viewed using iTOL. All positions with less than 95% site coverage were eliminated (partial deletion option). The trees with the highest log likelihood and bootstrap values from 1000 replications are shown in the figures.

Protein topologies of FLYC1 and FLYC2 (FIGS. 2B and 2C) were predicted using Proffer. DmOSCA topology (FIG. 2E) was determined by aligning the protein against Arabidopsis OSCA1.2 and assigning TM (transmembrane) and pore domains at the same positions identified in the OSCA1.2 protein structure. To model the Venus flytrap FLYC1 TM6 putative pore domain, residues A555-P593 were threaded to residues Q92-F130 of MscS in a closed conformation (PDB 2OAU). C7 symmetry was imposed using Rosettascripts, and side chain and backbone conformations were minimized using the Rosetta energy function with the solvation term turned off due to exposed hydrophobics in the partial structure.

### Accession Numbers

RNA-Seq data, the de novo transcriptome, ORF (open reading frame) identification, and downstream differential gene expression analysis can be found at NCBI using the accession numbers referenced above, and under the umbrella Bioproject PRJNA530242. CDS sequences subcloned and sequenced from plant cDNA template are available in GenBank (DmFLYC1, DcFLYC1.1, and DcFLYC1.2).

Cell Culture and Transient Transfection with Heterologous Polynucleotides

PIEZO1-knockout Human Embryonic Kidney 293T (HEK-P1KO) cells were used for all heterologous expression experiments. HEK-P1KO cells were generated using CRISPR-Cas9 nuclease genome editing technique as described previously (Lukacs, V. et al., 2015, Nat Commun, 6, 8329. doi:10.1038/ncomms9329), and were negative for mycoplasma contamination. Cells were grown in Dulbecco's Modified Eagle Medium (DMEM) containing 4.5 mg·ml−1 glucose, 10% fetal bovine serum, 50 units·ml−1 penicillin and 50 μg·ml−1 streptomycin. Cells were plated onto 12-mm round glass poly-D-lysine coated coverslips placed in 24-well plates and transfected using lipofectamine 2000 (Invitrogen) according to the manufacturer's instructions. All plasmids were transfected at a concentration of 700 ng·ml−1. Cells were recorded from 24 to 48 hours after transfection.

### Electrophysiology

Patch-clamp experiments in cells were performed in standard cell-attached, or excised patch (inside-out) mode using Axopatch 200B amplifier (Axon Instruments). Currents were sampled at 20 kHz and filtered at 2 kHz or 10 kHz. Leak currents before mechanical stimulations were subtracted off-line from the current traces. Voltages were not corrected for a liquid junction potential (LJP) except for ion selectivity experiments. LJP was calculated using Clampex 10.6 software. All experiments were performed at room temperature.

### Solutions

For cell-attached patch clamp recordings, external solution used to zero the membrane potential contained (in mM) 140 KCl, 1 MgCl₂, 10 glucose and 10 HEPES (pH 7.3 with KOH). Recording pipettes were of 1-3 MΩ resistance when filled with standard solution composed of (in mM) 130 mM NaCl, 5 KCl, 1 CaCl₂, 1 MgCl₂, 10 TEA-Cl and 10 HEPES (pH 7.3 with NaOH).

Ion selectivity experiments were performed in inside-out patch configurations. PCl/PNa was measured in extracellular solution composed of (in mM) 150 NaCl and 10 HEPES (pH 7.3 with NaOH) and intracellular solution was composed of (in mM) 30 NaCl, 10 HEPES and 225 Sucrose (pH 7.3 with NaOH). Calcium-gluconate solution was composed of (in mM) 50 Calcium gluconate, 0.5 CaCl₂, 10 HEPES, 170 Sucrose (pH 7.3 with NaOH).

### Mechanical Stimulation

Macroscopic stretch-activated currents were recorded in the cell-attached or excised, inside-out patch clamp configuration. Membrane patches were stimulated with 1 second negative pulses through the recording electrode using Clampex controlled pressure clamp HSPC-1 device (ALA-scientific), with inter-sweep duration of 40 seconds. Stretch-activated single-channel currents were recorded in the cell-attached configuration. Since single-channel amplitude is independent of the pressure intensity, the most optimal pressure stimulation was used to elicit responses that allowed single-channel amplitude measurements. These stimulation values were largely dependent on the number of channels in a given patch of the recording cell. Single-channel amplitude at a given potential was measured from trace histograms of 5 to 10 repeated recordings. Histograms were fitted with Gaussian equations using Clampfit 10.6 software. Single-channel slope conductance for each individual cell was calculated from linear regression curve fit to single-channel I-V plots.

### Permeability Ratio Measurements

Reversal potential for each cell in the mentioned solution was determined by interpolation of the respective current-voltage data. Permeability ratios were calculated by using the following Goldman-Hodgkin-Katz (GHK) equations:

Pc1/Pna Ratios:

$${E}_{rev} = \frac{RT}{F} ln \frac{{{P}_{Na} [ Na ]}_{o} + {{P}_{Cl} [ Cl ]}_{i}}{{{P}_{Na} [ Na ]}_{i} + {{P}_{Cl} [ Cl ]}_{o}}$$

### In Situ Hybridization and Imaging

Whole Venus flytrap and Cape sundew leaves were cut from the plant, fresh frozen in liquid N₂, and 15 μm sections collected. RNA in situ hybridization (RNAscope) was performed on sections according to manufacturer's instructions (ACDBio: #323100) using probes against DmFLYC1 (ACDBio; Ref: 546471, lot: 18177B), DmFLYC1-sense (ACDBio; Ref: 566181-C2, lot: 18361A), DmFLYC2 (ACDBio; Ref: 546481, lot: 18177C), DmOSCA (ACDBio; Ref: 571691, lot: 19032A), DcFLYC1.1/DcFLYC1.2 (ACDBio; Ref: 572451, lot: 19037B), and EF1α (ACDBio; Ref: 559911-C2, lot: 18311B). DmFLYC1-sense (FIG. 3B) and DmFLYC2 (FIGS. 9A and 9B) were tested on the same section. However, DmFLYC2 probe was independently tested in two additional experiments and no signal was observed. Slides were mounted with Vectashield+DAPI (Ref: H1200, lot: ZE0815). Stained sections were imaged with a Nikon C2 laser scanning confocal microscope and z-stacks were acquired through the entire section with a 60× objective. Displayed images are max projections of the entire z-stack. Images were processed using ImageJ (Fiji image processing package). Bungarotoxin binding sequence (BBS) targeted immunostaining experiments.

Labeling efficiency of DmFLYC1 was first tested with the intent of using a similar strategy for DmFLYC2, DmOSCA, and DcFLYC1.1/1.2. Protein expression at the membrane can be estimated by inserting a bungarotoxin binding sequence (BBS) in putative extracellular loops of the proteins of interest, and immunolabelling with α-bungarotoxin conjugated to a fluorescent tag and measuring fluorescence efficiency using flow cytometry. The 13 amino acid bungarotoxin binding sequence (WRYYESSLEPYPD; SEQ ID NO: 41) was cloned-in using Q5 site-directed mutagenesis kit in at the sites G216, P287, V289, or P292 in DmFLYC1-ires mCherry. PIEZO1-BBS-ires GFP (green fluorescent protein) construct, which has been demonstrated to be expressed at the membrane, was used as a positive control. HEK-P1 KO cells were transfected with PIEZO1-BBS, and FLYC1-BBS constructs. 48 hours post transfection, media was aspirated and cells were resuspended in 100 μL labeling buffer (PBS containing 2% Fetal bovine serum and 1 mM EDTA) using versene to detach cells from the plate. Cells were incubated with 1:100 Alexa Fluor 647-conjugated α-bungarotoxin for PIEZO1 and Alexa Fluor 488-conjugated α-bungarotoxin for DmFLYC1 (Thermo Fisher Scientific, Ref B13422, Ref B35450) in 1004 labeling buffer for 15 minutes at room temperature. Cells were then washed 3× with labeling buffer with a 5 minute incubation at each step and subjected to flow cytometry (LSR II). Labelling efficiency was measured using Flowjo. While positive labeling of PIEZO1-BBS was observed, labelling of FLYC1 was not observed at four distinct sites in two different putative extracellular loops of the protein. Insertion of the BBS in FLYC1 may have affected folding and trafficking of the wild-type protein.

### Example 7: Venus Fly Trap Plants are Sensitive to Ultrasound Stimuli

In land plants and green algae, chloride-permeable channel opening is associated with membrane depolarization due to the efflux of chloride ions down their electrochemical gradients. To ascertain whether plants had mechanosensitive chloride-channels that could be triggered by ultrasound stimuli, the well-known carnivorous plant, Dionaea muscipula (Venus fly trap), was selected for experimentation. The leaf of the Venus fly trap consists of an open bilobed trap, which attracts animal prey (insect) by volatile secretions. When an animal contacts one of the 3-4 mechanosensory hairs on each lobe, it bends the hair, which, in turn, generates an action potential in the sensory cells at the base of that hair and propagates throughout the trap. Two action potentials within a short 30 second time window lead to rapid closure, ensnaring the prey. This trap closes in 100 msecs, one of the fastest movements in the plant kingdom.

In Examples 1-6 described supra, genes that are selectively expressed in trigger hairs were identified, including two transcripts with homology to the MSL class and one to the OSCA family. One of these three transcripts was highly enriched in mechanosensitive trigger hairs and was termed FLYC1 (flycatcher 1). Example 4 supra demonstrates that expressing human codon-optimized FLYC1 in mechanically insensitive HEK-P1KO cells (A. E. Dubin et al., 2017, Neuron, 94:266-270. E263) rendered them sensitive to membrane stretch, thus confirming that this channel is chloride-selective.

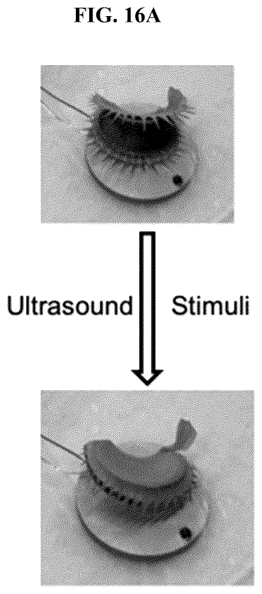

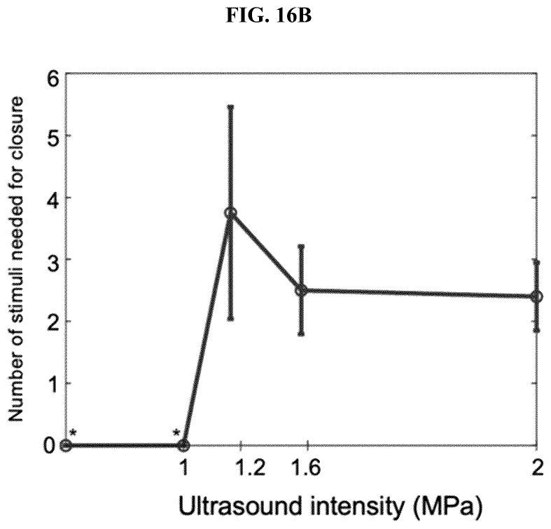

To test the sensitivity of Venus fly trap plants to ultrasound stimuli, the plants were mounted on a lithium niobate transducer, which allowed for delivery of different frequencies of ultrasound stimuli and to monitor responses of the plant. It was found that the plants' traps closed when at least two pulses of ultrasound stimuli of >1 MPa were delivered within 30 milliseconds (FIGS. 16A and 16B). These data confirmed that ultrasound stimuli could trigger the mechanosensory hairs, resulting in closure.

### Example 8: Exogenous Expression Establishing FLYC1 as a Candidate Sono-Silence Channel

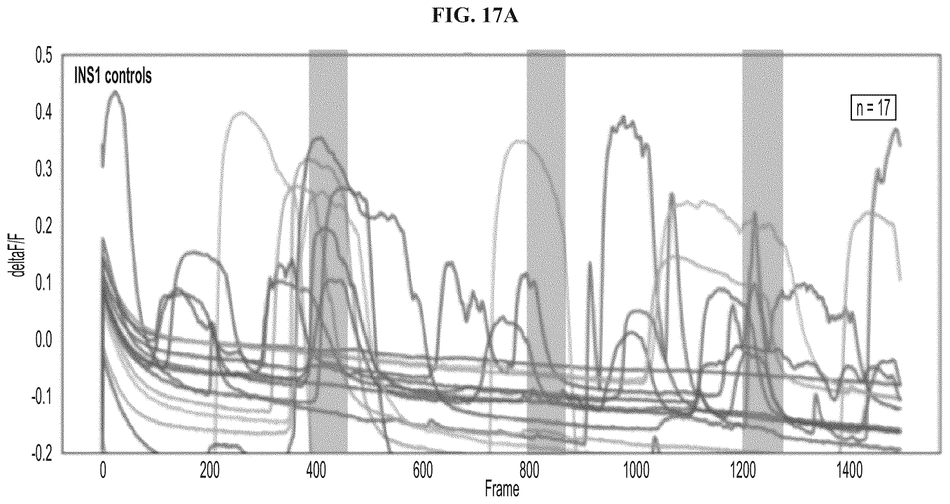

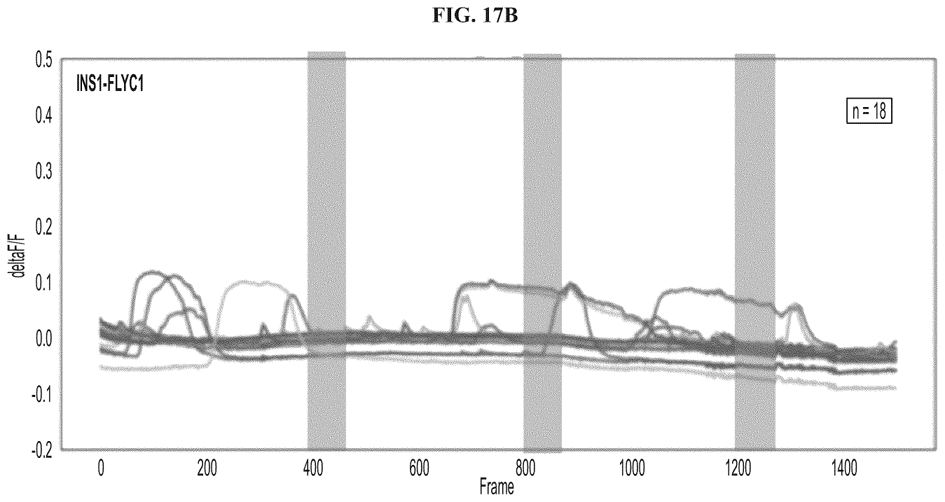

As described supra, FLYC1 is a chloride-selective mechanosensitive protein that is highly enriched in the trigger hairs of the Venus fly trap. An immortalized rat pancreatic beta cell line (INS1), (I. Cosar-Castellano et al., 2008, Diabetes, 57:3056-3068) was used to express the genetically encoded calcium indicator, GCaMP6 (T. W. Chen et al., Nature, 499:295-300). These cells exhibited spontaneous calcium events as measured by changes in GCaMP6 fluorescence. A human codon-optimized polynucleotide sequence encoding the FLYC1 polypeptide was used to transduce INS1 cells, which expressed the heterologous FLYC1 polypeptide. The heterologous FLYC1 polypeptide-expressing INS' cells were found to have reduced spontaneous responses following ultrasound stimulation (FIGS. 17A and 17B). In addition, the morphology of the INS' cells was not altered by FLYC1 alteration or ultrasound stimulation. The results of these experiments suggest that FLYC1 polypeptide may function as a sono-silencing channel when expressed as a heterologous protein in another cell type, such as an INS1 cell.

### Example 9: Fluorescence Imaging Plate Reader Membrane-Potential Assay

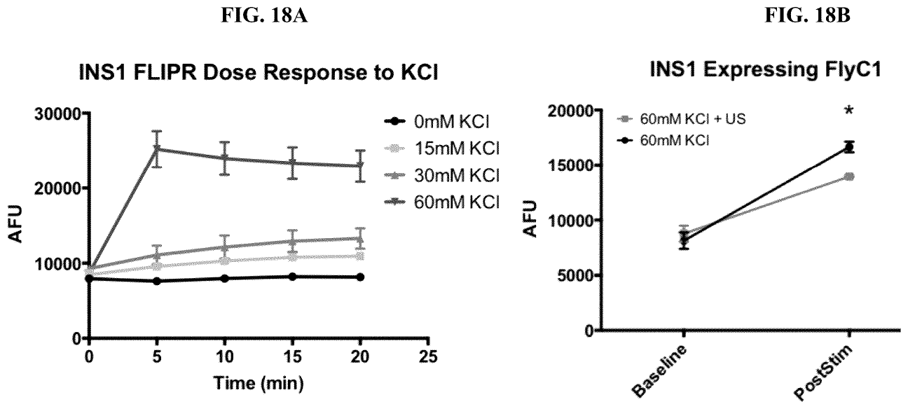

A plate reader-based fluorescence imaging plate reader (FLIPR) membrane-potential assay (FMP assay) was used to confirm that the FLYC1 polypeptide functioned as an ion channel when expressed as a heterologous (or exogenous) polypeptide in a given cell. The FMP (FLIPR membrane potential) kits provide proprietary assays developed by Molecular Dynamics for use in GPCR (G-protein-coupled receptor) and ion channel discovery by those skilled in the art. INS1 cells molecularly engineered to express a heterologous FLYC1 polypeptide were cultured in a 96-well plate. The FMP dye (FLIPR membrane potential dye) was added to the wells, and a baseline (t=0) reading was obtained from the plate reader. Different amounts of potassium chloride were added to the wells and changes in FMP (FLIPR membrane potential) fluorescence were monitored every five minutes for up to 20 minutes. Consistent with the manufacturer's handbook, a dose-dependent increase in fluorescence was observed with a peak response of an approximately 2.5 fold change to 60 mM KCl. INS1 cells expressing the heterologous FLYC1 polypeptide showed a lower response to KCl stimulation (FIGS. 18A and 18B). FIGS. 17A, 17B, 18A and 18B also show that heterologously expressed FLYC1 in INS-1 cells can suppress INS-1 cell activity, leading to less insulin secretion.

### Example 10: Sono-Silencing Characteristics

To determine the sono-silencing characteristics of the FLYC1 polypeptide exogenously expressed in HEK cells, each cell expressing a heterologous FLYC1 polypeptide was held at +60 mV, and various ultrasound pulse duration stimuli were delivered to the cells using 6.91 MHz transducers. The cells were held at +60 mV in order to observe maximal hyperpolarizing responses, as the equilibrium potential for chloride is approximately −70 mV. Lithium niobate transducers were used to deliver ultrasound stimuli to the cells. It was found that HEK cells expressing the FLYC1 polypeptide responded variably to ultrasound stimuli of different durations while the intensity was kept constant (2 MPa). Additionally, it was observed that the HEK cells which expressed the FLYC1 polypeptide displayed some voltage-dependency at depolarizing membrane potentials (FIG. 19). As demonstrated by FIG. 19, the FLYC1 expressed in INS-1 cells was able to inhibit excitable cells.

### Example 11: Calcium Imaging Analyses

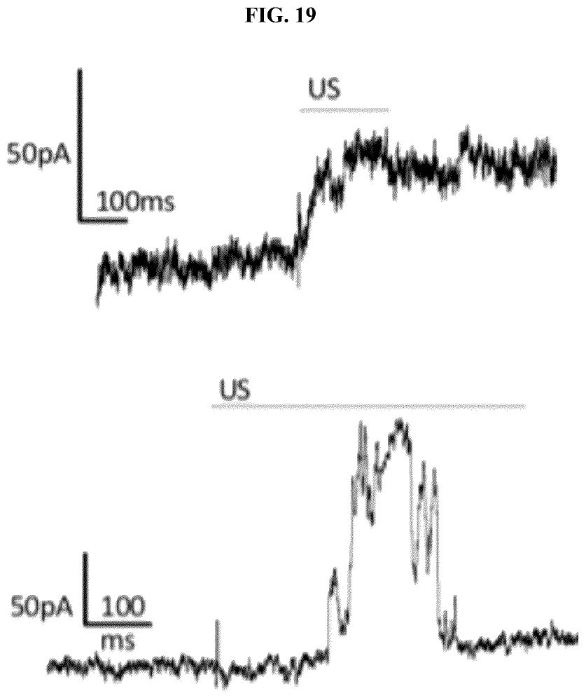

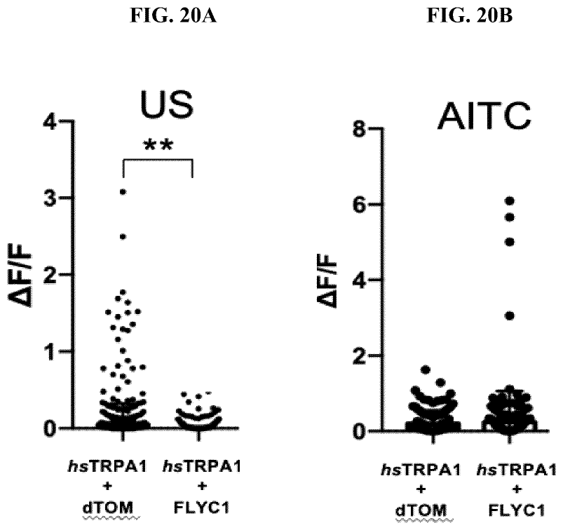

Calcium imaging was used to assess whether the FLYC1 polypeptide expressed in cells was able to suppress ultrasound activation of hsTRPA1. For these experiments, imaging techniques were employed in which an ultrasound transducer was aligned with a cell culture dish holder, and changes in cellular responses were recorded as changes in GCaMP fluorescence. Without intending to be bound by theory, because ultrasound activation of hsTRPA1 requires amplification from membrane-bound calcium channels, the simultaneous entry of chloride ions (from the expression of FLYC1 polypeptide) was expected to attenuate this response. The results showed that expression of the FLYC1 polypeptide in the cells consistently suppressed ultrasound (US)-evoked, but not AITC-activated, TRPA1 responses (FIGS. 20A and 20B). AITC is a chemical agonist that activates TRPA1 via an ultrasound-independent mechanism. (M. Raisinghani et al., 2011, J. Physiol Cell Physiol, 301:C587-600 and M. Duque et al., 2020, bioRxiv: 2020.2010.2014.338699.) As established by FIGS. 20A and 20B, the expression of the exogenous FLYC1 polypeptide is inhibitory by suppressing ultrasound-evoked activation of hsTRPA1-expressing cells.

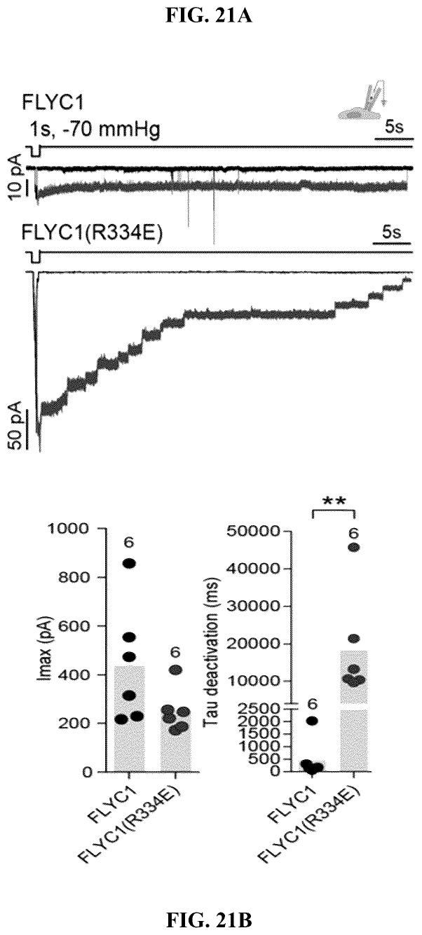

In addition, an R334E variant of the FLYC1 polypeptide was identified that exhibited slower inactivation kinetics in response to membrane stretch when expressed in cells (e.g., HEK-P1KO cells (FIGS. 21A and 21B). The R334E FLYC1 variant polypeptide sequence is provided in SEQ ID NO: 42. The expression of the FLYC1 variant polypeptide in cells confirmed that the FLYC1 polypeptide may be altered to obtain channels that exhibit different functional properties.

### OTHER EMBODIMENTS

From the foregoing description, it will be apparent that variations and modifications may be made to the various aspects and embodiments as described herein for adapting to various usages and conditions. Such embodiments are also within the scope of the following claims.

The recitation of a listing of elements in any definition of a variable herein includes definitions of that variable as any single element or combination (or subcombination) of listed elements. The recitation of an embodiment herein includes that embodiment as any single embodiment or in combination with any other embodiments or portions thereof.

All patents and publications mentioned in this specification are herein incorporated by reference to the same extent as if each independent patent and publication were specifically and individually indicated to be incorporated by reference.

What is claimed is:

1. A method of inducing cation or anion influx or efflux in a cell, the method comprising:
expressing in the cell a heterologous, mechanosensory polypeptide selected from the group consisting of DmFLYC1, DmFLYC2, DcFLYC1.1, DcFLYC1.2, and DmOSCA, or a variant thereof; and
applying ultrasound to the cell, thereby inducing cation or anion influx or efflux in the cell.

2. The method of claim 1, wherein the cell is sensitized to mechanical deformation or stretch caused by ultrasound.

3. The method of claim 1, wherein the application of ultrasound effects a change in mechanosensory polypeptide conductance in the cell and modulates a cell activity and/or function.

4. The method of claim 1, wherein the polypeptide comprises a sequence having at least 85% sequence identity to a polypeptide sequence selected from SEQ ID NOs: 5, 7, 9, 11, 13 or 42; or is encoded by a sequence having at least 85% sequence identity to a polynucleotide sequence selected from SEQ ID NOs: 6, 8, 10, 12, or 14.

5. A method for initiating or inducing a cellular response to mechanical deformation or stretch caused by ultrasound, the method comprising:
(a) transducing a cell to express a heterologous, mechanosensory polypeptide selected from DmFLYC1, DmFLYC2, DcFLYC1.1, DcFLYC1.2, and DmOSCA, or a variant thereof;
(b) applying ultrasound to the cell; and
(c) inducing cation or anion influx or efflux in the mechanosensory polypeptide expressing cell and an alteration in cell activity and/or function following the application of ultrasound, thereby initiating a cellular response to mechanical deformation or stretch caused by ultrasound.

6. The method of claim 1, wherein the polypeptide is encoded by a polynucleotide sequence codon-optimized for expression in a mammalian or human cell and is non-naturally occurring.

7. The method of claim 1, wherein the polypeptide is expressed in the cell following transduction of the cell by a plasmid or viral vector comprising a polynucleotide sequence encoding the polypeptide.

8. The method of claim 7, wherein the cell is transduced by a viral vector selected from a lentivirus vector or an adeno-associated virus (AAV) vector.

9. The method of claim 1, wherein the cell is one or more of a muscle cell, a cardiac muscle cell, an insulin secreting cell, a pancreatic cell, a kidney cell, or a neuronal cell.

10. The method of claim 1, wherein the ultrasound has a frequency of about 0.2 MHz to about 20 MHz.

11. The method of claim 1, wherein the ultrasound has a focal zone of about 1 cubic millimeter to about 1 cubic centimeter.

12. The method of claim 1, further comprising contacting the cell with a microbubble prior to applying ultrasound.

13. The method of claim 1, wherein the cell is in vitro, ex vivo, or in vivo.

14. The method of claim 1, wherein the cell is in a subject.

15. A plasmid or viral vector comprising a polynucleotide encoding a mechanosensory polypeptide selected from DmFLYC1, DmFLYC2, DcFLYC1.1, DcFLYC1.2, DmOSCA, or a variant thereof.

16. The plasmid or viral vector of claim 15, wherein the viral vector is a lentivirus vector or an adeno-associated virus (AAV) vector.

17. A cell comprising the plasmid or viral vector of claim 15 or a heterologous gene sequence encoding a polypeptide selected from the group consisting of DmFLYC1, DmFLYC2, DcFLYC1.1, DcFLYC1.2, and DmOSCA, or a variant thereof.

18. The cell of claim 17, wherein the cell is one or more of a muscle cell, a cardiac muscle cell, an insulin secreting cell, a pancreatic cell, a kidney cell, or a neuronal cell.

19. The cell of claim 17, wherein the cell is a plant cell.

20. An isolated polynucleotide encoding a mechanosensory polypeptide or a variant thereof selected from the group consisting of DmFLYC1, DmFLYC2, DcFLYC1.1, DcFLYC1.2, DmOSCA.

21. A mechanosensory polypeptide encoded by the polynucleotide of claim 20.

22. A plasmid or viral vector comprising the isolated polynucleotide of claim 20.

23. A cell comprising the isolated polynucleotide of claim 20.

24. A cell expressing the mechanosensory polypeptide of claim 21.

25. A composition comprising the cell of claim 23.
